(19) United States
(12) Patent Application Publication
(10) Pub. No.: US 2010/0256153 A1
(43) Pub. Date: Oct. 7, 2010

(54) METHODS FOR PREVENTING OR REDUCING RISK OF MORTALITY

(75) Inventors: Robert Frederich, Princeton, NJ (US); Jay Edelberg, Princeton, NJ (US); Frederick T. Fiedorek, Princeton, NJ (US)

Correspondence Address: McDonnell Boehnen Hulbert & Berghoff LLP;Bristol-Myers Squibb, 300 South Wacker Drive, Chicago, IL, 60606, US

(73) Assignees: BRISTOL-MYERS SQUIBB COMPANY, Princeton, NJ (US); ASTRAZENECA UK LIMITED, London (GB)

(21) Appl. No.: 12/748,140

(22) Filed: Mar. 26, 2010

Related U.S. Application Data
(60) Provisional application No. 61/164,153, filed on Mar. 27, 2009.
(60) Provisional application No. 61/165,399, filed on Mar. 31, 2009.

(51) Int. Cl. A61K 31/4985; A61K 31/403; A61K 31/40; A61P 9/12; A61P 3/06; A61P 9/00

(52) U.S. Cl. 514/249; 514/412; 514/423

(57) ABSTRACT

The present invention relates to methods for preventing or reducing the risk of mortality by any means including, but not limited to, cardiovascular events in mammals, particularly humans, comprising administering a dipeptidyl peptidase 4 (DPP-IV) inhibitor to the mammal or human. In addition, the present invention relates to methods for preventing or reducing the risk of non-fatal myocardial infarction and/or non-fatal stroke in mammals, particularly humans, comprising administering a DPP-IV inhibitor to the mammal or human.

This application claims the benefit of U.S. Provisional Application No. 61/164,153 filed Mar. 27, 2009, and U.S. Provisional Application No. 61/165,399 filed Mar. 31, 2009, both provisional applications are herein incorporated by reference for any purpose.

### TECHNICAL FIELD

The present invention relates to methods for preventing or reducing risk of mortality by any means including, but not limited to, a cardiovascular event, in mammals, particularly humans, comprising administering a dipeptidyl peptidase 4 (DPP-IV) inhibitor to the mammal or human. The present invention also relates to methods for preventing or reducing the risk of non-fatal myocardial infarction and/or non-fatal stroke in mammals, particularly humans, comprising administering a DPP-IV inhibitor to the mammal or human.

### BACKGROUND OF THE INVENTION

Diabetes mellitus, often referred to simply as diabetes, is a syndrome of disordered metabolism, usually due to a combination of hereditary and environmental causes, resulting in abnormally high blood sugar levels (hyperglycemia). Blood glucose levels are controlled by a complex interaction of multiple chemicals and hormones in the body, including the hormone insulin made in the beta cells of the pancreas. Diabetes refers to the group of diseases that lead to high blood glucose levels due to defects in either insulin secretion or insulin action in the body. The World Health Organization projects that the number of diabetics will exceed 350 million by 2030.

Diabetes is associated with an elevated risk of cardiovascular (CV) disease and is a leading cause of morbidity and mortality in diabetic patients. The American Academy of Physician Assistants reports that adults with diabetes have heart disease death rates about 2 to 4 times higher than adults without diabetes. The risk for stroke is 2 to 4 times higher among people with diabetes. About 65% of deaths among people with diabetes are due to heart disease and stroke. DPP-IV inhibitors are a known class of drugs that are useful for treating diabetes. Unfortunately, the use of pharmaceuticals may sometimes present a level of risk that outweighs the benefits of treatment. Accordingly, there is a need for pharmaceuticals, such as diabetes drugs, that have favorable safety profiles. Specifically, there is a need for therapies that control glucose levels in diabetic patients without increasing their risk of mortality by any means including, but not limited to, cardiovascular events.

### SUMMARY OF THE INVENTION

Unexpectedly, a class of antihyperglycemic agents, dipeptidyl peptidase 4 (DPP-IV) inhibitors, has been found to prevent or reduce the risk of death in diabetic human patients. They have also been found to prevent or reduce the risk of CV death and major adverse cardiac events (MACE) in a diabetic population. This beneficial effect does not appear to be related to the glucose lowering effects of the DPP-IV inhibitors and, therefore, this class of drugs can also be used to treat non-diabetic populations for the prevention of death and/or major adverse cardiac events.

The present invention relates to methods of preventing or reducing the risk of all cause mortality in mammals, particularly humans, comprising administering to the mammal or human, in need of such treatment, a therapeutically effective amount of a DPP-IV inhibitor, or a pharmaceutically acceptable salt thereof, optionally in combination with a different anti-diabetic agent, and optionally at least one pharmaceutically acceptable carrier. In certain embodiments, the present invention relates to the use of a DPP-IV inhibitor, or a pharmaceutically acceptable salt thereof, optionally in combination with a different anti-diabetic agent, and optionally at least one pharmaceutically acceptable carrier for the preparation or manufacture of a medicament for preventing or reducing the risk of mortality caused by a cardiovascular event in mammals, particularly humans. The preferred DPP-IV inhibitor useful in the methods of the present invention is saxagliptin. Metformin is the preferred anti-diabetic agent for combination therapies with saxagliptin for preventing or reducing the risk of mortality caused by a cardiovascular event in mammals, particularly humans.

The present invention relates to methods of preventing or reducing the risk of mortality caused by a cardiovascular event in mammals, particularly humans, comprising administering to the mammal or human, in need of such treatment, a therapeutically effective amount of a DPP-IV inhibitor, or a pharmaceutically acceptable salt thereof, optionally in combination with a different anti-diabetic agent, and optionally at least one pharmaceutically acceptable carrier. In certain embodiments, the present invention relates to the use of a DPP-IV inhibitor, or a pharmaceutically acceptable salt thereof, optionally in combination with a different anti-diabetic agent, and optionally at least one pharmaceutically acceptable carrier for the preparation or manufacture of a medicament for preventing or reducing the risk of mortality caused by a cardiovascular event in mammals, particularly humans. The preferred DPP-IV inhibitor useful in the methods of the present invention is saxagliptin. Metformin is the preferred anti-diabetic agent for combination therapies with saxagliptin for preventing or reducing the risk of mortality caused by a cardiovascular event in mammals, particularly humans.

The present invention relates to methods of preventing or reducing the risk of non-fatal myocardial infarction and/or non-fatal stroke in mammals, particularly humans, comprising administering to the mammal or human, in need of such treatment, a therapeutically effective amount of a DPP-IV inhibitor, or a pharmaceutically acceptable salt thereof, optionally in combination with a different anti-diabetic agent, and optionally at least one pharmaceutically acceptable carrier. In certain embodiments, the present invention relates to the use of a DPP-IV inhibitor, or a pharmaceutically acceptable salt thereof, optionally in combination with a different anti-diabetic agent, and optionally at least one pharmaceutically acceptable carrier for the preparation or manufacture of a medicament for preventing or reducing non-fatal myocardial infarction and/or non-fatal stroke in mammals, particularly humans. The preferred DPP-IV inhibitor useful in the methods of the present invention is saxagliptin. Metformin is the preferred anti-diabetic agent for combination therapies with saxagliptin for preventing or reducing the risk of non-fatal myocardial infarction and/or non-fatal stroke in mammals, particularly humans.

In another aspect, the present invention provides methods of preventing or reducing the risk of mortality caused by a second cardiovascular event in mammals, particularly humans, that have survived a first cardiovascular event comprising administering to the mammal or human, in need of such treatment, a therapeutically effective amount of a DPP-IV inhibitor, or a pharmaceutically acceptable salt thereof, optionally in combination with a different anti-diabetic agent, and optionally at least one pharmaceutically acceptable carrier. In certain embodiments, the present invention relates to the use of a DPP-IV inhibitor, or a pharmaceutically acceptable salt thereof, and optionally at least one pharmaceutically acceptable carrier for the preparation or manufacture of a medicament for preventing or reducing the risk of mortality caused by a second cardiovascular event in mammals, particularly humans, that have survived a first cardiovascular event. The preferred DPP-IV inhibitor useful in the methods of the present invention is saxagliptin. Metformin is the preferred anti-diabetic agent for combination therapies with saxagliptin for preventing or reducing the risk of mortality caused by a second cardiovascular event in mammals, particularly humans.

In another aspect, the present invention provides methods of preventing or reducing the risk of mortality caused by a third cardiovascular event in mammals, particularly humans, that have survived two cardiovascular events comprising administering to the mammal or human, in need of such treatment, a therapeutically effective amount of a DPP-IV inhibitor, or a pharmaceutically acceptable salt thereof, optionally in combination with a different anti-diabetic agent, and optionally at least one pharmaceutically acceptable carrier. In certain embodiments, the present invention relates to the use of a DPP-IV inhibitor, or a pharmaceutically acceptable salt thereof, and optionally at least one pharmaceutically acceptable carrier for the preparation or manufacture of a medicament for preventing or reducing the risk of mortality caused by a third cardiovascular event in mammals, particularly humans, that have survived two cardiovascular events. The preferred DPP-IV inhibitor useful in the methods of the present invention is saxagliptin. Metformin is the preferred anti-diabetic agent for combination therapies with saxagliptin for preventing or reducing the risk of mortality caused by a third cardiovascular event in mammals, particularly humans.

In another aspect, the present invention provides methods of preventing or reducing the risk of mortality caused by a cardiovascular event in mammals, particularly humans, that have type I or type II diabetes mellitus comprising administering to the mammal or human, in need of such treatment, a therapeutically effective amount of a DPP-IV inhibitor, or a pharmaceutically acceptable salt thereof, optionally in combination with a different anti-diabetic agent, and optionally at least one pharmaceutically acceptable carrier. In certain embodiments, the present invention relates to the use of a DPP-IV inhibitor, or a pharmaceutically acceptable salt thereof, and optionally at least one pharmaceutically acceptable carrier for the preparation or manufacture of a medicament for preventing or reducing the risk of mortality in mammals, particularly humans, that have type I or type II diabetes mellitus. The preferred DPP-IV inhibitor useful in the methods of the present invention is saxagliptin. Metformin is the preferred anti-diabetic agent for combination therapies with saxagliptin for preventing or reducing the risk of mortality caused by a cardiovascular event in mammals, particularly humans, that have type I or type II diabetes mellitus.

In another aspect, the present invention provides methods of preventing or reducing the risk of mortality caused by a cardiovascular event in mammals, particularly humans, that have a history of CV disease, a history of hypertension, a history of cholesterolemia, and/or a smoking history (current/previous) comprising administering to the mammal or human, in need of such treatment, a therapeutically effective amount of a DPP-IV inhibitor, or a pharmaceutically acceptable salt thereof, optionally in combination with a different anti-diabetic agent, and optionally at least one pharmaceutically acceptable carrier. In certain embodiments, the present invention relates to the use of a DPP-IV inhibitor, or a pharmaceutically acceptable salt thereof, and optionally at least one pharmaceutically acceptable carrier for the preparation or manufacture of a medicament for preventing or reducing the risk of mortality in mammals, particularly humans, that have a history of CV disease, a history of hypertension, a history of cholesterolemia, and/or a smoking history (current/previous). The preferred DPP-IV inhibitor useful in the methods of the present invention is saxagliptin. Metformin is the preferred anti-diabetic agent for combination therapies with saxagliptin for preventing or reducing the risk of mortality caused by a cardiovascular event in mammals, particularly humans, that have a history of CV disease, a history of hypertension, a history of cholesterolemia, and/or a smoking history (current/previous).

In another aspect, the present invention provides methods of preventing or reducing the risk of mortality caused by a cardiovascular event in mammals, particularly humans, that have mixed dyslipidemia, a smoking history (current/previous), or coronary heart disease comprising administering to the mammal or human, in need of such treatment, a therapeutically effective amount of a DPP-IV inhibitor, or a pharmaceutically acceptable salt thereof, optionally in combination with a different anti-diabetic agent, and optionally at least one pharmaceutically acceptable carrier. In certain embodiments, the present invention relates to the use of a DPP-IV inhibitor, or a pharmaceutically acceptable salt thereof, and optionally at least one pharmaceutically acceptable carrier for the preparation or manufacture of a medicament for preventing or reducing the risk of mortality in mammals, particularly humans, that have mixed dyslipidemia, a smoking history (current/previous), or coronary heart disease. The preferred DPP-IV inhibitor useful in the methods of the present invention is saxagliptin. Metformin is the preferred anti-diabetic agent for combination therapies with saxagliptin for preventing or reducing the risk of mortality caused by a cardiovascular event in mammals, particularly humans, that have mixed dyslipidemia, a smoking history (current/previous), or coronary heart disease.

In another aspect, the present invention provides the use of pharmaceutical compositions. It is to be understood that the methods of the present invention, as described by each of the embodiments herein, includes pharmaceutical compositions comprised of at least one pharmaceutically acceptable carrier.

Specific embodiments of this invention will become evident from the following more detailed description of certain preferred embodiments and the claims.

### BRIEF DESCRIPTION OF THE DRAWINGS

FIG. 1 is a graphical illustration of the proportion or percentage of subjects in two groups who died or suffered a MACE (aka CV death/MI/stroke) over time in the pooled saxagliptin clinical studies. Patients administered saxagliptin daily (2.5 mg, 5.0 mg, or 10.0 mg) represent the ALL SAXA group and patients administered placebo or metformin represent the control group. The standard Kaplan-Meier analytical technique was followed which takes into account that some subjects dropped out of the study or had not reached the final time point (128 weeks).

FIG. 2 is a graphical illustration of the relative incidence of CV death and all-cause death (death from any cause) for patients administered saxagliptin (2.5 mg, 5.0 mg, or 10.0 mg) and patients administered placebo or metformin, represented as a ratio of saxagliptin:control. Three methods (Cox, Exact, and Mantel-Haenszel) were used for determining the saxagliptin:control ratio.

FIG. 3 is a graphical illustration of particular subgroups of subjects, from the Phase 2b/3 pooled population, with an increased risk of having a MACE event. Subjects with a history of CV disease, at least one CV risk factor (in addition to T2DM); at least two CV risk factors (in addition to T2DM); a history of hypertension; or a history of hypercholesterolemia, and males had lower incidences of primary MACE events when administered saxagliptin as compared to control.

FIG. 4 is a graphical illustration, in terms of hazard ratios and corresponding 95% confidence intervals, of reduced risk of primary MACE events in subjects at higher risk of having a MACE event. The analysis was conducted using the Cox Proportional Hazards model.

FIG. 5 is a graphical illustration of the incidence of a CV events (aka ACE), Investigator-CV death/MI/stroke (Inv-CV death/MI/Stroke aka MACE), and CEC-CV death/MI/Stroke (CEC-Adjudicated CV events) for patients administered saxagliptin (2.5 mg, 5.0 mg, or 10.0 mg) and patients administered placebo or metformin, represented as a ratio of saxagliptin:control. Four methods (Cox, Incidence Rate Ratio by Exact, and Mantel-Haenszel, and Incidence Ratio) were used for determining the saxagliptin:control ratio.

FIG. 6 is a graphical illustration of the incidence of a CV event (stroke, MI, CV death) for patients administered saxagliptin ((2.5 mg, 5.0 mg, or 10.0 mg) and patients administered placebo or metformin, represented as a ratio of saxagliptin:control for the pooled analysis and the individual studies which comprise the pooled analysis. The analysis is the incidence rate ratio with Baysian 95% credibility interval.

FIG. 7 is a graphical illustration of non-diabetic male rat survival over a two year period following administration of saxagliptin (25 mg/kg/day) or one of two cohorts of placebo (water).

FIG. 8 is a graphical illustration of non-diabetic female rat survival over a two year period following administration of saxagliptin (25 mg/kg/day) or one of two cohorts of placebo (water).

FIG. 9 is a graphical illustration of non-diabetic female mouse survival over a two year period following administration of saxagliptin (50 mg/kg/day) or one of two cohorts of placebo (water).

FIG. 10 is a graphical illustration of non-diabetic male mouse survival over a two year period following administration of saxagliptin (50 mg/kg/day) or one of two cohorts of placebo (water).

FIG. 11 is a graphical illustration comparing point estimates and 95% confidence interval of reduced incidence of mortality for saxagliptin, sitagliptin, and vildagliptin for pooled studies using the Fischer Exact Test analysis.

FIG. 12 is a graphical illustration comparing point estimates and 95% confidence interval of reduced incidence of ischemic serious adverse (heart or brain related) events (SAE) for saxagliptin, sitagliptin, and vildagliptin for pooled studies using the Fischer Exact Test analysis.

FIG. 13 is a graphical illustration of the relative risk of heart attack for subjects administered sitagliptin relative to metformin, propensity adjusted.

### DETAILED DESCRIPTION OF THE INVENTION

The present invention relates to methods of preventing or reducing the risk of all cause mortality in mammals, particularly humans, comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of all cause mortality range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce all cause mortality in patients with type II diabetes mellitus (T2DM), and in animals without diabetes.

In another aspect, the present invention relates to methods of preventing or reducing the risk of all cause mortality in mammals, particularly humans, that have a history of CV disease, a history of hypertension, a history of hypercholesterolemia, and/or a smoking history (current/previous) comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of all cause mortality range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of all cause mortality in T2DM patients that have a history of CV disease, a history of hypertension, a history of hypercholesterolemia, and/or a smoking history (current/previous).

In another aspect, the present invention relates to methods of preventing or reducing the risk of mortality caused by a cardiovascular event in mammals, particularly humans, comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of mortality caused by a CV event range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce risk of mortality caused by a CV event in patients with type II diabetes mellitus (T2DM).

In another aspect, the present invention relates to methods of preventing or reducing the risk of mortality caused by a cardiovascular event in mammals, particularly humans, that have a history of CV disease comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of mortality caused by a CV event range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of mortality caused by a CV event in T2DM patients that have a history of cardiovascular disease.

In another aspect, the present invention relates to methods of preventing or reducing the risk of mortality caused by a cardiovascular event in mammals, particularly humans, that have a history of hypertension comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of mortality caused by a CV event range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of mortality caused by a CV event in T2DM patients that have a history of hypertension.

In another aspect, the present invention relates to methods of preventing or reducing the risk of mortality caused by a cardiovascular event in mammals, particularly humans, that have a history of hypercholesterolemia comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of mortality caused by a CV event range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of mortality caused by a CV event in T2DM patients that have a history of hypercholesterolemia.

In another aspect, the present invention relates to methods of preventing or reducing the risk of mortality caused by a cardiovascular event in mammals, particularly humans, that have a current or previous smoking history comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of mortality caused by a CV event range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of mortality caused by a CV event in T2DM patients that have a current or previous smoking history.

In another aspect, the present invention relates to methods of preventing or reducing the risk of non-fatal myocardial infarction in mammals, particularly humans, comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of non-fatal myocardial infarction range from about 0.5 mgs/day to about 400 mgs/day. The preferred saxagliptin doses are 2.5 mgs/day, 5 mgs/day, and 10 mgs/day Therapeutically effective amounts of saxagliptin prevent or reduce non-fatal myocardial infarction in T2DM patients.

In another aspect, the present invention relates to methods of preventing or reducing the risk of non-fatal myocardial infarction in mammals, particularly humans, that have a history of CV disease comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of non-fatal myocardial infarction range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of non-fatal myocardial infarction in T2DM patients that have a history of CV disease.

In another aspect, the present invention relates to methods of preventing or reducing the risk of non-fatal myocardial infarction in mammals, particularly humans, that have a history of hypertension comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of non-fatal myocardial infarction range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of non-fatal myocardial infarction in T2DM patients that have a history of hypertension.

In another aspect, the present invention relates to methods of preventing or reducing the risk of non-fatal myocardial infarction in mammals, particularly humans, that have a history of hypercholesterolemia comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of non-fatal myocardial infarction range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of non-fatal myocardial infarction in T2DM patients that have a history of hypercholesterolemia.

In another aspect, the present invention relates to methods of preventing or reducing the risk of non-fatal myocardial infarction in mammals, particularly humans, that have a current or previous smoking history comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of non-fatal myocardial infarction range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of non-fatal myocardial infarction in T2DM patients that have a current or previous smoking history.

In another aspect, the present invention relates to methods of preventing or reducing the risk of non-fatal stroke in mammals, particularly humans, comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of non-fatal stroke range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of non-fatal stroke in T2DM patients.

In another aspect, the present invention relates to methods of preventing or reducing the risk of non-fatal stroke in mammals, particularly humans, that have a history of CV disease comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of non-fatal stroke range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of non-fatal stroke in T2DM patients that have a history of CV disease.

In another aspect, the present invention relates to methods of preventing or reducing the risk of non-fatal stroke in mammals, particularly humans, that have a history of hypertension comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of non-fatal stroke range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of non-fatal stroke in T2DM patients that have a history of hypertension.

In another aspect, the present invention relates to methods of preventing or reducing the risk of non-fatal stroke in mammals, particularly humans, that have a history of hypercholesterolemia comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of non-fatal stroke range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of non-fatal stroke in T2DM patients that have a history of hypercholesterolemia.

In another aspect, the present invention relates to methods of preventing or reducing the risk of non-fatal stroke in mammals, particularly humans, that have a current or previous smoking history comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of non-fatal stroke range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of non-fatal stroke in T2DM patients that have a current or previous smoking history.

In another aspect, the present invention relates to methods of preventing or reducing the risk of mortality caused by a second cardiovascular event in mammals, particularly humans, that have survived a first cardiovascular event comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of mortality caused by a second CV event range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of mortality caused by a second CV event in T2DM patients that have survived a first CV event.

In another aspect, the present invention relates to methods of preventing or reducing the risk of mortality caused by a third cardiovascular event in mammals, particularly humans, that have survived two cardiovascular events comprising administering to the mammal or human, in need of such treatment, a therapeutically effective amount of a saxagliptin or a pharmaceutically acceptable salt, hydrate, or hydrate of a salt, thereof, and optionally at least one pharmaceutically acceptable carrier. Therapeutically effective amounts of saxagliptin for preventing or reducing the risk of mortality caused by a third CV event range from about 0.5 mgs/day to about 400 mgs/day. The preferred saxagliptin doses are 2.5 mgs/day, 5 mgs/day, and 10 mgs/day. Therapeutically effective amounts of saxagliptin prevent or reduce the risk of mortality caused by a third CV event in T2DM patients that have survived two CV events.

In another aspect, the present invention provides a method of prolonging the survival time following a first cardiovascular event in mammals, particularly humans, comprising administering to the mammal or human, in need of such treatment, a therapeutically effective amount of saxagliptin or a pharmaceutically acceptable salt, hydrate, or hydrate of a salt, thereof, and optionally at least one pharmaceutically acceptable carrier. In a further aspect, the present invention provides a method of increasing the time interval between a first cardiovascular event and a second cardiovascular event in mammals, particularly humans, that have survived a first cardiovascular event comprising administering to the mammal or human, in need of such treatment, a therapeutically effective amount of saxagliptin, or a pharmaceutically acceptable salt, hydrate, or hydrate of a salt, thereof, and optionally at least one pharmaceutically acceptable carrier. Therapeutically effective amounts of saxagliptin range from about 0.5 mgs/day to about 400 mgs/day. The preferred saxagliptin doses are 2.5 mgs/day, 5 mgs/day, and 10 mgs/day.

In another aspect, the present invention provides a method of prolonging the survival time following a second cardiovascular event in mammals, particularly humans, comprising administering to the mammal or human, in need of such treatment, a therapeutically effective amount of saxagliptin, or a pharmaceutically acceptable salt, hydrate, or hydrate of a salt, thereof, and optionally at least one pharmaceutically acceptable carrier. In a further aspect, the present invention provides a method of increasing the time interval between a second cardiovascular event and a third cardiovascular event in mammals, particularly humans, that have survived a second cardiovascular event comprising administering to the mammal or human, in need of such treatment, a therapeutically effective amount of saxagliptin, or a pharmaceutically acceptable salt, hydrate, or hydrate of a salt, thereof, and optionally at least one pharmaceutically acceptable carrier. Therapeutically effective amounts of saxagliptin range from about 0.5 mgs/day to about 400 mgs/day. The preferred saxagliptin doses are 2.5 mgs/day, 5 mgs/day, and 10 mgs/day.

In another aspect, the present invention relates to methods of preventing or reducing all cause mortality in mammals, particularly humans, comprising administering to the mammal or human, in need of such treatment, a therapeutically effective amount of a combination of saxagliptin, or a pharmaceutically acceptable salt, hydrate, or hydrate of a salt, thereof, a sulfonylurea, and optionally at least one pharmaceutically acceptable carrier. Therapeutically effective amounts of saxagliptin for combination with a sulfonylurea range from about 0.5 mgs/day to about 400 mgs/day. The preferred saxagliptin doses for combination with a sulfonylurea are 2.5 mgs/day, 5 mgs/day, and 10 mgs/day. Therapeutically effective amounts of the combination of saxagliptin and a sulfonylurea prevent or reduce risk of all cause mortality in T2DM patients.

In another aspect, the present invention relates to methods of preventing or reducing the risk of all cause mortality in mammals, particularly humans, that have a history of CV disease, a history of hypertension, a history of hypercholesterolemia, and/or a smoking history (current/previous) comprising administering to the mammal or human, in need of such treatment, a therapeutically effective amount of a combination of saxagliptin, or a pharmaceutically acceptable salt, hydrate, or hydrate of a salt, thereof, a sulfonylurea, and optionally at least one pharmaceutically acceptable carrier. Therapeutically effective amounts of saxagliptin for combination with a sulfonylurea range from about 0.5 mgs/day to about 400 mgs/day. The preferred saxagliptin doses for combination with a sulfonylurea are 2.5 mgs/day, 5 mgs/day, and 10 mgs/day. Therapeutically effective amounts of the combination of saxagliptin and a sulfonylurea prevent or reduce risk of all cause mortality in T2DM patients that have a history of cardiovascular disease, a history of hypertension, a history of hypercholesterolemia, and/or a smoking history (current/previous).

In another aspect, the present invention relates to methods of preventing or reducing the risk of mortality caused by a cardiovascular event in mammals, particularly humans, comprising administering to the mammal or human, in need of such treatment, a therapeutically effective amount of a combination of saxagliptin, or a pharmaceutically acceptable salt, hydrate, or hydrate of a salt, thereof, a sulfonylurea, and optionally at least one pharmaceutically acceptable carrier. Therapeutically effective amounts of saxagliptin for combination with a sulfonylurea range from about 0.5 mgs/day to about 400 mgs/day. The preferred saxagliptin doses for combination with a sulfonylurea are 2.5 mgs/day, 5 mgs/day, and 10 mgs/day. Therapeutically effective amounts of the combination of saxagliptin and a sulfonylurea prevent or reduce risk of mortality caused by a cardiovascular event in T2DM patients.

In another aspect, the present invention relates to methods of preventing or reducing the risk of mortality caused by a cardiovascular event in mammals, particularly humans, that have a history of CV disease, a history of hypertension, a history of hypercholesterolemia, and/or a smoking history (current/previous) comprising administering to the mammal or human, in need of such treatment, a therapeutically effective amount of a combination of saxagliptin, or a pharmaceutically acceptable salt, hydrate, or hydrate of a salt, thereof, a sulfonylurea, and optionally at least one pharmaceutically acceptable carrier. Therapeutically effective amounts of saxagliptin for combination with a sulfonylurea range from about 0.5 mgs/day to about 400 mgs/day. The preferred saxagliptin doses for combination with a sulfonylurea are 2.5 mgs/day, 5 mgs/day, and 10 mgs/day. Therapeutically effective amounts of the combination of saxagliptin and a sulfonylurea prevent or reduce risk of mortality caused by a cardiovascular event in T2DM patients that have a history of CV disease, a history of hypertension, a history of hypercholesterolemia, and/or a smoking history (current/previous).

In another aspect, the present invention relates to methods of preventing or reducing the risk of non-fatal myocardial infarction and/or non-fatal stroke in mammals, particularly humans, comprising administering to the mammal or human, in need of such treatment, a therapeutically effective amount of a combination of saxagliptin, or a pharmaceutically acceptable salt, hydrate, or hydrate of a salt, thereof, a sulfonylurea, and optionally at least one pharmaceutically acceptable carrier. Therapeutically effective amounts of saxagliptin for combination with a sulfonylurea range from about 0.5 mgs/day to about 400 mgs/day. The preferred saxagliptin doses for combination with a sulfonylurea are 2.5 mgs/day, 5 mgs/day, and 10 mgs/day. Therapeutically effective amounts of the combination of saxagliptin and a sulfonylurea prevent or reduce risk of non-fatal myocardial infarction and/or non-fatal stroke in T2DM patients.

In another aspect, the present invention relates to methods of preventing or reducing the risk of non-fatal myocardial infarction and/or non-fatal stroke in mammals, particularly humans, that have a history of CV disease, a history of hypertension, a history of hypercholesterolemia, and/or a smoking history (current/previous) comprising administering to the mammal or human, in need of such treatment, a therapeutically effective amount of a combination of saxagliptin, or a pharmaceutically acceptable salt, hydrate, or hydrate of a salt, thereof, a sulfonylurea, and optionally at least one pharmaceutically acceptable carrier. Therapeutically effective amounts of saxagliptin for combination with a sulfonylurea range from about 0.5 mgs/day to about 400 mgs/day. The preferred saxagliptin doses for combination with a sulfonylurea are 2.5 mgs/day, 5 mgs/day, and 10 mgs/day. Therapeutically effective amounts of the combination of saxagliptin and a sulfonylurea prevent or reduce risk of non-fatal myocardial infarction and/or non-fatal stroke in T2DM patients that have a history of CV disease, a history of hypertension, a history of hypercholesterolemia, and/or a smoking history (previous/current).

In another aspect, the present invention relates to methods of preventing or reducing the risk of mortality caused by a second cardiovascular event in mammals, particularly humans, that have survived a first cardiovascular event comprising administering to the mammal or human, in need of such treatment, a therapeutically effective amount of a combination of saxagliptin or a pharmaceutically acceptable salt, hydrate, or hydrate of a salt, thereof, a sulfonylurea, and optionally at least one pharmaceutically acceptable carrier. Therapeutically effective amounts of saxagliptin for combination with a sulfonylurea for preventing or reducing the risk of mortality caused by a second CV event range from about 0.5 mgs/day to about 400 mgs/day. The preferred saxagliptin doses for combination with a sulfonylurea are 2.5 mgs/day, 5 mgs/day, and 10 mgs/day. Therapeutically effective amounts of the combination of saxagliptin and a sulfonylurea prevent or reduce the risk of mortality caused by a second CV event in T2DM patients that have survived a first CV event.

In another aspect, the present invention relates to methods of preventing or reducing the risk of mortality caused by a third cardiovascular event in mammals, particularly humans, that have survived two cardiovascular events comprising administering to the mammal or human, in need of such treatment, a therapeutically effective amount of a combination of saxagliptin or a pharmaceutically acceptable salt, hydrate, or hydrate of a salt, thereof, a sulfonylurea, and optionally at least one pharmaceutically acceptable carrier. Therapeutically effective amounts of saxagliptin for combination with a sulfonylurea for preventing or reducing the risk of mortality caused by a third CV event range from about 0.5 mgs/day to about 400 mgs/day. The preferred saxagliptin doses for combination with a sulfonylurea are 2.5 mgs/day, 5 mgs/day, and 10 mgs/day. Therapeutically effective amounts of the combination of saxagliptin and a sulfonylurea prevent or reduce the risk of mortality caused by a third CV event in T2DM patients that have survived two CV events.

In another aspect, the present invention provides a method of prolonging the survival time following a first cardiovascular event in mammals, particularly humans, comprising administering to the mammal or human, in need of such treatment, a therapeutically effective amount of a combination of saxagliptin or a pharmaceutically acceptable salt, hydrate, or hydrate of a salt, thereof, a sulfonylurea, and optionally at least one pharmaceutically acceptable carrier. In a further aspect, the present invention provides a method of increasing the time interval between a first cardiovascular event and a second cardiovascular event in mammals, particularly humans, that have survived a first cardiovascular event comprising administering to the mammal or human, in need of such treatment, a therapeutically effective amount of a combination of saxagliptin or a pharmaceutically acceptable salt, hydrate, or hydrate of a salt, thereof, a sulfonylurea, and optionally at least one pharmaceutically acceptable carrier. Therapeutically effective amounts of saxagliptin for combination with a sulfonylurea for prolonging the survival time following a first cardiovascular event or for increasing the time interval between a first cardiovascular event and a second cardiovascular range from about 0.5 mgs/day to about 400 mgs/day. The preferred saxagliptin doses for combination with a sulfonylurea are 2.5 mgs/day, 5 mgs/day, and 10 mgs/day.

In another aspect, the present invention provides a method of prolonging the survival time following a second cardiovascular event in mammals, particularly humans, comprising administering to the mammal or human, in need of such treatment, a therapeutically effective amount of a combination of saxagliptin or a pharmaceutically acceptable salt, hydrate, or hydrate of a salt, thereof, a sulfonylurea, and optionally at least one pharmaceutically acceptable carrier.

In a further aspect, the present invention provides a method of increasing the time interval between a second cardiovascular event and a third cardiovascular event in mammals, particularly humans, that have survived a second cardiovascular event comprising administering to the mammal or human, in need of such treatment, a therapeutically effective amount of saxagliptin or a pharmaceutically acceptable salt, hydrate, or hydrate of a salt, thereof, a sulfonylurea, and optionally at least one pharmaceutically acceptable carrier. Therapeutically effective amounts of saxagliptin for combination with a sulfonylurea for prolonging the survival time following a second cardiovascular event or for increasing the time interval between a second cardiovascular event and a third cardiovascular range from about 0.5 mgs/day to about 400 mgs/day. The preferred saxagliptin doses for combination with a sulfonylurea are 2.5 mgs/day, 5 mgs/day, and 10 mgs/day.

In another aspect, the present invention relates to methods of preventing or reducing the risk of all cause mortality in mammals, particularly humans, comprising administering to the mammal or human, in need of such treatment, a therapeutically effective amount of a combination of saxagliptin, or a pharmaceutically acceptable salt, hydrate, or hydrate of a salt, thereof, glyburide, or a pharmaceutically acceptable salt thereof, and optionally at least one pharmaceutically acceptable carrier. Therapeutically effective amounts of saxagliptin for combination with glyburide range from about 0.5 mgs/day to about 400 mgs/day. The preferred saxagliptin doses for combination with glyburide are 2.5 mgs/day, 5 mgs/day, and 10 mgs/day. The preferred dosing range for glyburide is about 0.5 mgs/day to about 15 mgs/day. Therapeutically effective amounts of the combination of saxagliptin and glyburide prevent or reduce risk of all cause mortality in T2DM patients.

In another aspect, the present invention relates to methods of preventing or reducing the risk of all cause mortality in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of saxagliptin, or a pharmaceutically acceptable salt, hydrate, or hydrate of a salt, thereof, glyburide, or a pharmaceutically acceptable salt thereof, and optionally at least one pharmaceutically acceptable carrier. Therapeutically effective amounts of saxagliptin for combination with glyburide range from about 0.5 mgs/day to about 400 mgs/day. The preferred saxagliptin doses for combination with glyburide are 2.5 mgs/day, 5 mgs/day, and 10 mgs/day. The preferred dosing range for glyburide is about 0.5 mgs/day to about 15 mgs/day. Therapeutically effective amounts of the combination of saxagliptin and glyburide prevent or reduce risk of all cause mortality in T2DM patients that have a history of CV disease, a history of hypertension, a history of hypercholesterolemia, and/or a smoking history (current/previous).

In another aspect, the present invention relates to methods of preventing or reducing the risk of mortality caused by a cardiovascular event in mammals, particularly humans, comprising administering to the mammal or human, in need of such treatment, a therapeutically effective amount of a combination of saxagliptin, or a pharmaceutically acceptable salt, hydrate, or hydrate of a salt, thereof, glyburide, or a pharmaceutically acceptable salt thereof, and optionally at least one pharmaceutically acceptable carrier. Therapeutically effective amounts of saxagliptin for combination with glyburide range from about 0.5 mgs/day to about 400 mgs/day. The preferred saxagliptin doses for combination with glyburide are 2.5 mgs/day, 5 mgs/day, and 10 mgs/day. The preferred dosing range for glyburide is about 0.5 mgs/day to about 15 mgs/day. Therapeutically effective amounts of the combination of saxagliptin and glyburide prevent or reduce risk of mortality caused by a CV event in T2DM patients.

In another aspect, the present invention relates to methods of preventing or reducing the risk of mortality caused by a cardiovascular event in mammals, particularly humans, that have a history of CV disease, a history of hypertension, a history of hypercholesterolemia, and/or a smoking history (current/previous) comprising administering to the mammal or human, in need of such treatment, a therapeutically effective amount of a combination of saxagliptin, or a pharmaceutically acceptable salt, hydrate, or hydrate of a salt, thereof, glyburide, or a pharmaceutically acceptable salt thereof, and optionally at least one pharmaceutically acceptable carrier. Therapeutically effective amounts of saxagliptin for combination with glyburide range from about 0.5 mgs/day to about 400 mgs/day. The preferred saxagliptin doses for combination with glyburide are 2.5 mgs/day, 5 mgs/day, and 10 mgs/day. The preferred dosing range for glyburide is about 0.5 mgs/day to about 15 mgs/day. Therapeutically effective amounts of the combination of saxagliptin and glyburide prevent or reduce risk of mortality caused by a CV event in T2DM patients that have a history of CV disease, a history of hypertension, a history of hypercholesterolemia, and/or a smoking history (current/previous).

In another aspect, the present invention relates to methods of preventing or reducing the risk of non-fatal myocardial infarction and/or non-fatal stroke in mammals, particularly humans, comprising administering to the mammal or human, in need of such treatment, a therapeutically effective amount of a combination of saxagliptin, or a pharmaceutically acceptable salt, hydrate, or hydrate of a salt, thereof, glyburide, or a pharmaceutically acceptable salt thereof, and optionally at least one pharmaceutically acceptable carrier. Therapeutically effective amounts of saxagliptin for combination with glyburide range from about 0.5 mgs/day to about 400 mgs/day. The preferred saxagliptin doses for combination with glyburide are 2.5 mgs/day, 5 mgs/day, and 10 mgs/day. The preferred dosing range for glyburide is about 0.5 mgs/day to about 15 mgs/day. Therapeutically effective amounts of the combination of saxagliptin and glyburide prevent or reduce risk of non-fatal myocardial infarction and/or non-fatal stroke in T2DM patients.

In another aspect, the present invention relates to methods of preventing or reducing the risk of non-fatal myocardial infarction and/or non-fatal stroke in mammals, particularly humans, that have a history of CV disease, a history of hypertension, a history of hypercholesterolemia, and/or a smoking history (current/previous) comprising administering to the mammal or human, in need of such treatment, a therapeutically effective amount of a combination of saxagliptin, or a pharmaceutically acceptable salt, hydrate, or hydrate of a salt, thereof, glyburide, or a pharmaceutically acceptable salt thereof, and optionally at least one pharmaceutically acceptable carrier. Therapeutically effective amounts of saxagliptin for combination with glyburide range from about 0.5 mgs/day to about 400 mgs/day. The preferred saxagliptin doses for combination with glyburide are 2.5 mgs/day, 5 mgs/day, and 10 mgs/day. The preferred dosing range for glyburide is about 0.5 mgs/day to about 15 mgs/day. Therapeutically effective amounts of the combination of saxagliptin and glyburide prevent or reduce risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to methods of preventing or reducing the risk of mortality caused by a second cardiovascular event in mammals, particularly humans, that have survived a first cardiovascular event comprising administering to the mammal or human, in need of such treatment, a therapeutically effective amount of a combination of saxagliptin or a pharmaceutically acceptable salt, hydrate, or hydrate of a salt, thereof, glyburide, or a pharmaceutically acceptable salt thereof, and optionally at least one pharmaceutically acceptable carrier. Therapeutically effective amounts of saxagliptin for combination with glyburide for preventing or reducing the risk of mortality caused by a second CV event range from about 0.5 mgs/day to about 400 mgs/day. The preferred saxagliptin doses for combination with glyburide are 2.5 mgs/day, 5 mgs/day, and 10 mgs/day. The preferred dosing range for glyburide is about 0.5 mgs/day to about 15 mgs/day. Therapeutically effective amounts of the combination of saxagliptin and glyburide prevent or reduce the risk of mortality caused by a second cardiovascular event in T2DM patients that have survived a first cardiovascular event.

In another aspect, the present invention relates to methods of preventing or reducing the risk of mortality caused by a third cardiovascular event in mammals, particularly humans, that have survived two cardiovascular events comprising administering to the mammal or human, in need of such treatment, a therapeutically effective amount of a combination of saxagliptin or a pharmaceutically acceptable salt, hydrate, or hydrate of a salt, thereof, glyburide, or a pharmaceutically acceptable salt thereof, and optionally at least one pharmaceutically acceptable carrier. Therapeutically effective amounts of saxagliptin for combination with glyburide for preventing or reducing the risk of mortality caused by a third cardiovascular event range from about 0.5 mgs/day to about 400 mgs/day. The preferred saxagliptin doses for combination with glyburide are 2.5 mgs/day, 5 mgs/day, and 10 mgs/day. The preferred dosing range for glyburide is about 0.5 mgs/day to about 15 mgs/day. Therapeutically effective amounts of the combination of saxagliptin and glyburide prevent or reduce the risk of mortality caused by a third cardiovascular event in T2DM patients that have survived two cardiovascular events.

In another aspect, the present invention provides a method of prolonging the survival time following a first cardiovascular event in mammals, particularly humans, comprising administering to the mammal or human, in need of such treatment, a therapeutically effective amount of a combination of saxagliptin or a pharmaceutically acceptable salt, hydrate, or hydrate of a salt, thereof, glyburide, or a pharmaceutically acceptable salt thereof, and optionally at least one pharmaceutically acceptable carrier. In a further aspect, the present invention provides a method of increasing the time interval between a first cardiovascular event and a second cardiovascular event in mammals, particularly humans, that have survived a first cardiovascular event comprising administering to the mammal or human, in need of such treatment, a therapeutically effective amount of a combination of saxagliptin or a pharmaceutically acceptable salt, hydrate, or hydrate of a salt, thereof, glyburide, or a pharmaceutically acceptable salt thereof, and optionally at least one pharmaceutically acceptable carrier. Therapeutically effective amounts of saxagliptin for combination with glyburide range from about 0.5 mgs/day to about 400 mgs/day. The preferred saxagliptin doses for combination with glyburide are 2.5 mgs/day, 5 mgs/day, and 10 mgs/day. The preferred dosing range for glyburide is about 0.5 mgs/day to about 15 mgs/day.

In another aspect, the present invention provides a method of prolonging the survival time following a second cardiovascular event in mammals, particularly humans, comprising administering to the mammal or human, in need of such treatment, a therapeutically effective amount of a combination of saxagliptin or a pharmaceutically acceptable salt, hydrate, or hydrate of a salt, thereof, glyburide, or a pharmaceutically acceptable salt thereof, and optionally at least one pharmaceutically acceptable carrier. In a further aspect, the present invention provides a method of increasing the time interval between a second cardiovascular event and a third cardiovascular event in mammals, particularly humans, that have survived a second cardiovascular event comprising administering to the mammal or human, in need of such treatment, a therapeutically effective amount of saxagliptin or a pharmaceutically acceptable salt, hydrate, or hydrate of a salt, thereof, glyburide, or a pharmaceutically acceptable salt thereof, and optionally at least one pharmaceutically acceptable carrier. Therapeutically effective amounts of saxagliptin for combination with glyburide range from about 0.5 mgs/day to about 400 mgs/day. The preferred saxagliptin doses for combination with glyburide are 2.5 mgs/day, 5 mgs/day, and 10 mgs/day. The preferred dosing range for glyburide is about 0.5 mgs/day to about 15 mgs/day.

In another aspect, the present invention relates to methods of preventing or reducing the risk of all cause mortality in mammals, particularly humans, comprising administering to the mammal or human, in need of such treatment, a therapeutically effective amount of a combination of saxagliptin, or a pharmaceutically acceptable salt, hydrate, or hydrate of a salt, thereof, a biguanide, and optionally at least one pharmaceutically acceptable carrier. Therapeutically effective amounts of saxagliptin for combination with a biguanide range from about 0.5 mgs/day to about 400 mgs/day. The preferred saxagliptin doses for combination with a biguanide are 2.5 mgs/day, 5 mgs/day, and 10 mgs/day. Therapeutically effective amounts of the combination of saxagliptin and a biguanide prevent or reduce risk of all cause mortality in T2DM patients.

In another aspect, the present invention relates to methods of preventing or reducing the risk of all cause mortality in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of saxagliptin, or a pharmaceutically acceptable salt, hydrate, or hydrate of a salt, thereof, a biguanide, and optionally at least one pharmaceutically acceptable carrier. Therapeutically effective amounts of saxagliptin for combination with a biguanide range from about 0.5 mgs/day to about 400 mgs/day. The preferred saxagliptin doses for combination with a biguanide are 2.5 mgs/day, 5 mgs/day, and 10 mgs/day. Therapeutically effective amounts of the combination of saxagliptin and a biguanide prevent or reduce risk of all cause mortality in T2DM patients that have a history of CV disease, a history of hypertension, a history of hypercholesterolemia, and/or a smoking history (current/previous).

In another aspect, the present invention relates to methods of preventing or reducing the risk of mortality caused by a cardiovascular event in mammals, particularly humans, comprising administering to the mammal or human, in need of such treatment, a therapeutically effective amount of a combination of saxagliptin, or a pharmaceutically acceptable salt, hydrate, or hydrate of a salt, thereof, a biguanide, and optionally at least one pharmaceutically acceptable carrier. Therapeutically effective amounts of saxagliptin for combination with a biguanide range from about 0.5 mgs/day to about 400 mgs/day. The preferred saxagliptin doses for combination with a biguanide are 2.5 mgs/day, 5 mgs/day, and 10 mgs/day. Therapeutically effective amounts of the combination of saxagliptin and a biguanide prevent or reduce risk of mortality caused by a CV event in T2DM patients.

In another aspect, the present invention relates to methods of preventing or reducing the risk of mortality caused by a cardiovascular event in mammals, particularly humans, that have a history of CV disease, a history of hypertension, a history of hypercholesterolemia, and/or a smoking history (current/previous) comprising administering to the mammal or human, in need of such treatment, a therapeutically effective amount of a combination of saxagliptin, or a pharmaceutically acceptable salt, hydrate, or hydrate of a salt, thereof, a biguanide, and optionally at least one pharmaceutically acceptable carrier. Therapeutically effective amounts of saxagliptin for combination with a biguanide range from about 0.5 mgs/day to about 400 mgs/day. The preferred saxagliptin doses for combination with a biguanide are 2.5 mgs/day, 5 mgs/day, and 10 mgs/day. Therapeutically effective amounts of the combination of saxagliptin and a biguanide prevent or reduce risk of mortality caused by a CV event in T2DM patients that have a history of CV disease, a history of hypertension, a history of hypercholesterolemia, and/or a smoking history (current/previous).

In another aspect, the present invention relates to methods of preventing or reducing the risk of non-fatal myocardial infarction and/or non-fatal stroke in mammals, particularly humans, comprising administering to the mammal or human, in need of such treatment, a therapeutically effective amount of a combination of saxagliptin, or a pharmaceutically acceptable salt, hydrate, or hydrate of a salt, thereof, a biguanide, and optionally at least one pharmaceutically acceptable carrier. Therapeutically effective amounts of saxagliptin for combination with a biguanide range from about 0.5 mgs/day to about 400 mgs/day. The preferred saxagliptin doses for combination with a biguanide are 2.5 mgs/day, 5 mgs/day, and 10 mgs/day. Therapeutically effective amounts of the combination of saxagliptin and a biguanide prevent or reduce risk of non-fatal myocardial infarction and/or non-fatal stroke in T2DM patients.

In another aspect, the present invention relates to methods of preventing or reducing the risk of non-fatal myocardial infarction and/or non-fatal stroke in mammals, particularly humans, that have a history of CV disease, a history of hypertension, a history of hypercholesterolemia, and/or a smoking history (current/previous) comprising administering to the mammal or human, in need of such treatment, a therapeutically effective amount of a combination of saxagliptin, or a pharmaceutically acceptable salt, hydrate, or hydrate of a salt, thereof, a biguanide, and optionally at least one pharmaceutically acceptable carrier. Therapeutically effective amounts of saxagliptin for combination with a biguanide range from about 0.5 mgs/day to about 400 mgs/day. The preferred saxagliptin doses for combination with a biguanide are 2.5 mgs/day, 5 mgs/day, and 10 mgs/day. Therapeutically effective amounts of the combination of saxagliptin and a biguanide prevent or reduce risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to methods of preventing or reducing the risk of mortality caused by a second cardiovascular event in mammals, particularly humans, that have survived a first cardiovascular event comprising administering to the mammal or human, in need of such treatment, a therapeutically effective amount of a combination of saxagliptin or a pharmaceutically acceptable salt, hydrate, or hydrate of a salt, thereof, a biguanide, and optionally at least one pharmaceutically acceptable carrier. Therapeutically effective amounts of saxagliptin for combination with a biguanide for preventing or reducing the risk of mortality caused by a second CV event range from about 0.5 mgs/day to about 400 mgs/day. The preferred saxagliptin doses for combination with a biguanide are 2.5 mgs/day, 5 mgs/day, and 10 mgs/day. Therapeutically effective amounts of the combination of saxagliptin and a biguanide prevent or reduce the risk of mortality caused by a second CV event T2DM patients that have survived a first CV event.

In another aspect, the present invention relates to methods of preventing or reducing the risk of mortality caused by a third cardiovascular event in mammals, particularly humans, that have survived two cardiovascular events comprising administering to the mammal or human, in need of such treatment, a therapeutically effective amount of a combination of saxagliptin or a pharmaceutically acceptable salt, hydrate, or hydrate of a salt, thereof, a biguanide, and optionally at least one pharmaceutically acceptable carrier. Therapeutically effective amounts of saxagliptin for combination with a biguanide for preventing or reducing the risk of mortality caused by a third CV event range from about 0.5 mgs/day to about 400 mgs/day. The preferred saxagliptin doses for combination with a biguanide are 2.5 mgs/day, 5 mgs/day, and 10 mgs/day. Therapeutically effective amounts of the combination of saxagliptin and a biguanide prevent or reduce the risk of mortality caused by a third CV event in T2DM patients that have survived two CV events.

In another aspect, the present invention provides a method of prolonging the survival time following a first cardiovascular event in mammals, particularly humans, comprising administering to the mammal or human, in need of such treatment, a therapeutically effective amount of a combination of saxagliptin or a pharmaceutically acceptable salt, hydrate, or hydrate of a salt, thereof, a biguanide, and optionally at least one pharmaceutically acceptable carrier. In a further aspect, the present invention provides a method of increasing the time interval between a first cardiovascular event and a second cardiovascular event in mammals, particularly humans, that have survived a first cardiovascular event comprising administering to the mammal or human, in need of such treatment, a therapeutically effective amount of a combination of saxagliptin or a pharmaceutically acceptable salt, hydrate, or hydrate of a salt, thereof, a biguanide, and optionally at least one pharmaceutically acceptable carrier. Therapeutically effective amounts of saxagliptin for combination with a biguanide for prolonging the survival time following a first cardiovascular event or for increasing the time interval between a first cardiovascular event and a second cardiovascular range from about 0.5 mgs/day to about 400 mgs/day. The preferred saxagliptin doses for combination with a biguanide are 2.5 mgs/day, 5 mgs/day, and 10 mgs/day.

In another aspect, the present invention provides a method of prolonging the survival time following a second cardiovascular event in mammals, particularly humans, comprising administering to the mammal or human, in need of such treatment, a therapeutically effective amount of a combination of saxagliptin or a pharmaceutically acceptable salt, hydrate, or hydrate of a salt, thereof, a biguanide, and optionally at least one pharmaceutically acceptable carrier. In a further aspect, the present invention provides a method of increasing the time interval between a second cardiovascular event and a third cardiovascular event in mammals, particularly humans, that have survived a second cardiovascular event comprising administering to the mammal or human, in need of such treatment, a therapeutically effective amount of a combination of saxagliptin or a pharmaceutically acceptable salt, hydrate, or hydrate of a salt, thereof, a biguanide, and optionally at least one pharmaceutically acceptable carrier. Therapeutically effective amounts of saxagliptin for combination with a biguanide for prolonging the survival time following a second cardiovascular event or for increasing the time interval between a second cardiovascular event and a third cardiovascular range from about 0.5 mgs/day to about 400 mgs/day. The preferred saxagliptin doses for combination with a biguanide are 2.5 mgs/day, 5 mgs/day, and 10 mgs/day.

In another aspect, the present invention relates to methods of preventing or reducing the risk of all cause mortality in mammals, particularly humans, comprising administering to the mammal or human, in need of such treatment, a therapeutically effective amount of a combination of saxagliptin, or a pharmaceutically acceptable salt, hydrate, or hydrate of a salt, thereof, metformin, or a pharmaceutically acceptable salt thereof, and optionally at least one pharmaceutically acceptable carrier. Therapeutically effective amounts of saxagliptin for combination with metformin range from about 0.5 mgs/day to about 400 mgs/day. The preferred saxagliptin doses for combination with metformin are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Therapeutically effective amounts of the combination of saxagliptin and metformin prevent or reduce risk of all cause mortality in T2DM patients.

In another aspect, the present invention relates to methods of preventing or reducing the risk of all cause mortality in mammals, particularly humans, that have a history of CV disease, a history of hypertension, a history of hypercholesterolemia, and/or a smoking history (current/previous) comprising administering to the mammal or human, in need of such treatment, a therapeutically effective amount of a combination of saxagliptin, or a pharmaceutically acceptable salt, hydrate, or hydrate of a salt, thereof, metformin, or a pharmaceutically acceptable salt thereof, and optionally at least one pharmaceutically acceptable carrier. Therapeutically effective amounts of saxagliptin for combination with metformin range from about 0.5 mgs/day to about 400 mgs/day. The preferred saxagliptin doses for combination with metformin are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Therapeutically effective amounts of the combination of saxagliptin and metformin prevent or reduce risk of all cause mortality in T2DM patients that have a history of CV disease, a history of hypertension, a history of hypercholesterolemia, and/or a smoking history (current/previous).

In another aspect, the present invention relates to methods of preventing or reducing the risk of mortality caused by a cardiovascular event in mammals, particularly humans, comprising administering to the mammal or human, in need of such treatment, a therapeutically effective amount of a combination of saxagliptin, or a pharmaceutically acceptable salt, hydrate, or hydrate of a salt, thereof, metformin, or a pharmaceutically acceptable salt thereof, and optionally at least one pharmaceutically acceptable carrier. Therapeutically effective amounts of saxagliptin for combination with metformin range from about 0.5 mgs/day to about 400 mgs/day. The preferred saxagliptin doses for combination with metformin are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Therapeutically effective amounts of the combination of saxagliptin and metformin prevent or reduce risk of mortality caused by a CV event in T2DM patients.

In another aspect, the present invention relates to methods of preventing or reducing the risk of mortality caused by a cardiovascular event in mammals, particularly humans, that have a history of CV disease, a history of hypertension, a history of hypercholesterolemia, and/or a smoking history (current/previous) comprising administering to the mammal or human, in need of such treatment, a therapeutically effective amount of a combination of saxagliptin, or a pharmaceutically acceptable salt, hydrate, or hydrate of a salt, thereof, metformin, or a pharmaceutically acceptable salt thereof, and optionally at least one pharmaceutically acceptable carrier. Therapeutically effective amounts of saxagliptin for combination with metformin range from about 0.5 mgs/day to about 400 mgs/day. The preferred saxagliptin doses for combination with metformin are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Therapeutically effective amounts of the combination of saxagliptin and metformin prevent or reduce risk of mortality caused by a CV event in T2DM patients that have a history of CV disease, a history of hypertension, a history of hypercholesterolemia, and/or a smoking history (current/previous).

In another aspect, the present invention relates to methods of preventing or reducing the risk of non-fatal myocardial infarction and/or non-fatal stroke in mammals, particularly humans, comprising administering to the mammal or human, in need of such treatment, a therapeutically effective amount of a combination of saxagliptin, or a pharmaceutically acceptable salt, hydrate, or hydrate of a salt, thereof, metformin, or a pharmaceutically acceptable salt thereof, and optionally at least one pharmaceutically acceptable carrier. Therapeutically effective amounts of saxagliptin for combination with metformin range from about 0.5 mgs/day to about 400 mgs/day. The preferred saxagliptin doses for combination with metformin are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Therapeutically effective amounts of the combination of saxagliptin and metformin prevent or reduce risk of non-fatal myocardial infarction and/or non-fatal stroke in T2DM patients.

In another aspect, the present invention relates to methods of preventing or reducing the risk of non-fatal myocardial infarction and/or non-fatal stroke in mammals, particularly humans, that have a history of CV disease, a history of hypertension, a history of hypercholesterolemia, and/or a smoking history (current/previous) comprising administering to the mammal or human, in need of such treatment, a therapeutically effective amount of a combination of saxagliptin, or a pharmaceutically acceptable salt, hydrate, or hydrate of a salt, thereof, metformin, or a pharmaceutically acceptable salt thereof, and optionally at least one pharmaceutically acceptable carrier. Therapeutically effective amounts of saxagliptin for combination with metformin range from about 0.5 mgs/day to about 400 mgs/day. The preferred saxagliptin doses for combination with metformin are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Therapeutically effective amounts of the combination of saxagliptin and metformin prevent or reduce risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to methods of preventing or reducing the risk of mortality caused by a second cardiovascular event in mammals, particularly humans, that have survived a first cardiovascular event comprising administering to the mammal or human, in need of such treatment, a therapeutically effective amount of a combination of saxagliptin or a pharmaceutically acceptable salt, hydrate, or hydrate of a salt, thereof, metformin, or a pharmaceutically acceptable salt thereof, and optionally at least one pharmaceutically acceptable carrier. Therapeutically effective amounts of saxagliptin for combination with metformin for preventing or reducing the risk of mortality caused by a second CV event range from about 0.5 mgs/day to about 400 mgs/day. The preferred saxagliptin doses for combination with metformin are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Therapeutically effective amounts of the combination of saxagliptin and metformin prevent or reduce the risk of mortality caused by a second CV event in T2DM patients.

In another aspect, the present invention relates to methods of preventing or reducing the risk of mortality caused by a third cardiovascular event in mammals, particularly humans, that have survived two cardiovascular events comprising administering to the mammal or human, in need of such treatment, a therapeutically effective amount of a combination of saxagliptin or a pharmaceutically acceptable salt, hydrate, or hydrate of a salt, thereof, metformin, or a pharmaceutically acceptable salt thereof, and optionally at least one pharmaceutically acceptable carrier. Therapeutically effective amounts of saxagliptin for combination with metformin for preventing or reducing the risk of mortality caused by a third CV event range from about 0.5 mgs/day to about 400 mgs/day. The preferred saxagliptin doses for combination with metformin are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Therapeutically effective amounts of the combination of saxagliptin and metformin prevent or reduce the risk of mortality caused by a third CV event in T2DM patients that have survived two CV events.

In another aspect, the present invention provides a method of prolonging the survival time following a first cardiovascular event in mammals, particularly humans, comprising administering to the mammal or human, in need of such treatment, a therapeutically effective amount of a combination of saxagliptin or a pharmaceutically acceptable salt, hydrate, or hydrate of a salt, thereof, metformin, or a pharmaceutically acceptable salt thereof, and optionally at least one pharmaceutically acceptable carrier. In a further aspect, the present invention provides a method of increasing the time interval between a first cardiovascular event and a second cardiovascular event in mammals, particularly humans, that have survived a first cardiovascular event comprising administering to the mammal or human, in need of such treatment, a therapeutically effective amount of a combination of saxagliptin or a pharmaceutically acceptable salt, hydrate, or hydrate of a salt, thereof, metformin, or a pharmaceutically acceptable salt thereof, and optionally at least one pharmaceutically acceptable carrier. Therapeutically effective amounts of saxagliptin for combination with metformin range from about 0.5 mgs/day to about 400 mgs/day. The preferred saxagliptin doses for combination with metformin are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl.

In another aspect, the present invention provides a method of prolonging the survival time following a second cardiovascular event in mammals, particularly humans, comprising administering to the mammal or human, in need of such treatment, a therapeutically effective amount of a combination of saxagliptin or a pharmaceutically acceptable salt, hydrate, or hydrate of a salt, thereof, metformin, or a pharmaceutically acceptable salt thereof, and optionally at least one pharmaceutically acceptable carrier. In a further aspect, the present invention provides a method of increasing the time interval between a second cardiovascular event and a third cardiovascular event in mammals, particularly humans, that have survived a second cardiovascular event comprising administering to the mammal or human, in need of such treatment, a therapeutically effective amount of a combination of saxagliptin or a pharmaceutically acceptable salt, hydrate, or hydrate of a salt, thereof, metformin, or a pharmaceutically acceptable salt thereof, and optionally at least one pharmaceutically acceptable carrier. Therapeutically effective amounts of saxagliptin for combination with metformin range from about 0.5 mgs/day to about 400 mgs/day. The preferred saxagliptin doses for combination with metformin are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl.

In another aspect, the present invention relates to methods of preventing or reducing all cause mortality in mammals, particularly humans, comprising administering to the mammal or human, in need of such treatment, a therapeutically effective amount of a combination of saxagliptin, or a pharmaceutically acceptable salt, hydrate, or hydrate of a salt, thereof, a thiazolidinedione, and optionally at least one pharmaceutically acceptable carrier. Therapeutically effective amounts of saxagliptin for combination with a thiazolidinedione range from about 0.5 mgs/day to about 400 mgs/day. The preferred saxagliptin doses for combination with a thiazolidinedione are 2.5 mgs/day, 5 mgs/day, and 10 mgs/day. Therapeutically effective amounts of the combination of saxagliptin and a thiazolidinedione prevent or reduce risk of all cause mortality in T2DM patients.

In another aspect, the present invention relates to methods of preventing or reducing the risk of all cause mortality in mammals, particularly humans, that have a history of CV disease, a history of hypertension, a history of hypercholesterolemia, and/or a smoking history (current/previous) comprising administering to the mammal or human, in need of such treatment, a therapeutically effective amount of a combination of saxagliptin, or a pharmaceutically acceptable salt, hydrate, or hydrate of a salt, thereof, a thiazolidinedione, and optionally at least one pharmaceutically acceptable carrier. Therapeutically effective amounts of saxagliptin for combination with a thiazolidinedione range from about 0.5 mgs/day to about 400 mgs/day. The preferred saxagliptin doses for combination with a thiazolidinedione are 2.5 mgs/day, 5 mgs/day, and 10 mgs/day. Therapeutically effective amounts of the combination of saxagliptin and a thiazolidinedione prevent or reduce risk of all cause mortality in T2DM patients that have a history of CV disease, a history of hypertension, a history of hypercholesterolemia, and/or a smoking history (current/previous).

In another aspect, the present invention relates to methods of preventing or reducing the risk of mortality caused by a cardiovascular event in mammals, particularly humans, comprising administering to the mammal or human, in need of such treatment, a therapeutically effective amount of a combination of saxagliptin, or a pharmaceutically acceptable salt, hydrate, or hydrate of a salt, thereof, a thiazolidinedione, and optionally at least one pharmaceutically acceptable carrier. Therapeutically effective amounts of saxagliptin for combination with a thiazolidinedione range from about 0.5 mgs/day to about 400 mgs/day. The preferred saxagliptin doses for combination with a thiazolidinedione are 2.5 mgs/day, 5 mgs/day, and 10 mgs/day. Therapeutically effective amounts of the combination of saxagliptin and a thiazolidinedione prevent or reduce risk of mortality caused by a cardiovascular event in T2DM patients.

In another aspect, the present invention relates to methods of preventing or reducing the risk of mortality caused by a cardiovascular event in mammals, particularly humans, that have a history of CV disease, a history of hypertension, a history of hypercholesterolemia, and/or a smoking history (current/previous) comprising administering to the mammal or human, in need of such treatment, a therapeutically effective amount of a combination of saxagliptin, or a pharmaceutically acceptable salt, hydrate, or hydrate of a salt, thereof, a thiazolidinedione, and optionally at least one pharmaceutically acceptable carrier. Therapeutically effective amounts of saxagliptin for combination with a thiazolidinedione range from about 0.5 mgs/day to about 400 mgs/day. The preferred saxagliptin doses for combination with a thiazolidinedione are 2.5 mgs/day, 5 mgs/day, and 10 mgs/day. Therapeutically effective amounts of the combination of saxagliptin and a thiazolidinedione prevent or reduce risk of mortality caused by a CV event in T2DM patients that have a history of cardiovascular disease, a history of hypertension, a history of hypercholesterolemia, and/or a smoking history (current/previous).

In another aspect, the present invention relates to methods of preventing or reducing the risk of non-fatal myocardial infarction and/or non-fatal stroke in mammals, particularly humans, comprising administering to the mammal or human, in need of such treatment, a therapeutically effective amount of a combination of saxagliptin, or a pharmaceutically acceptable salt, hydrate, or hydrate of a salt, thereof, a thiazolidinedione, and optionally at least one pharmaceutically acceptable carrier. Therapeutically effective amounts of saxagliptin for combination with a thiazolidinedione range from about 0.5 mgs/day to about 400 mgs/day. The preferred saxagliptin doses for combination with a thiazolidinedione are 2.5 mgs/day, 5 mgs/day, and 10 mgs/day. Therapeutically effective amounts of the combination of saxagliptin and a thiazolidinedione prevent or reduce risk of non-fatal myocardial infarction and/or non-fatal stroke in T2DM patients.

In another aspect, the present invention relates to methods of preventing or reducing the risk of non-fatal myocardial infarction and/or non-fatal stroke in mammals, particularly humans, that have a history of CV disease, a history of hypertension, a history of hypercholesterolemia, and/or a smoking history (current/previous) comprising administering to the mammal or human, in need of such treatment, a therapeutically effective amount of a combination of saxagliptin, or a pharmaceutically acceptable salt, hydrate, or hydrate of a salt, thereof, a thiazolidinedione, and optionally at least one pharmaceutically acceptable carrier. Therapeutically effective amounts of saxagliptin for combination with a thiazolidinedione range from about 0.5 mgs/day to about 400 mgs/day. The preferred saxagliptin doses for combination with a thiazolidinedione are 2.5 mgs/day, 5 mgs/day, and 10 mgs/day. Therapeutically effective amounts of the combination of saxagliptin and a thiazolidinedione prevent or reduce risk of non-fatal myocardial infarction and/or non-fatal stroke in T2DM patients that have a history of cardiovascular disease, a history of hypertension, a history of hypercholesterolemia, and/or a smoking history (previous/current).

In another aspect, the present invention relates to methods of preventing or reducing the risk of mortality caused by a second cardiovascular event in mammals, particularly humans, that have survived a first cardiovascular event comprising administering to the mammal or human, in need of such treatment, a therapeutically effective amount of a combination of saxagliptin or a pharmaceutically acceptable salt, hydrate, or hydrate of a salt, thereof, a thiazolidinedione, and optionally at least one pharmaceutically acceptable carrier. Therapeutically effective amounts of saxagliptin for combination with a thiazolidinedione for preventing or reducing the risk of mortality caused by a second cardiovascular event range from about 0.5 mgs/day to about 400 mgs/day. The preferred saxagliptin doses for combination with a thiazolidinedione are 2.5 mgs/day, 5 mgs/day, and 10 mgs/day. Therapeutically effective amounts of the combination of saxagliptin and a thiazolidinedione prevent or reduce the risk of mortality caused by a second cardiovascular event in T2DM patients that have survived a first cardiovascular event.

In another aspect, the present invention relates to methods of preventing or reducing the risk of mortality caused by a third cardiovascular event in mammals, particularly humans, that have survived two previous cardiovascular events comprising administering to the mammal or human, in need of such treatment, a therapeutically effective amount of a combination of saxagliptin or a pharmaceutically acceptable salt, hydrate, or hydrate of a salt, thereof, a thiazolidinedione, and optionally at least one pharmaceutically acceptable carrier. Therapeutically effective amounts of saxagliptin for combination with a thiazolidinedione for preventing or reducing the risk of mortality caused by a third cardiovascular event range from about 0.5 mgs/day to about 400 mgs/day. The preferred saxagliptin doses for combination with a thiazolidinedione are 2.5 mgs/day, 5 mgs/day, and 10 mgs/day. Therapeutically effective amounts of the combination of saxagliptin and a thiazolidinedione prevent or reduce the risk of mortality caused by a third CV event in T2DM patients that have survived two previous cardiovascular events.

In another aspect, the present invention provides a method of prolonging the survival time following a first cardiovascular event in mammals, particularly humans, comprising administering to the mammal or human, in need of such treatment, a therapeutically effective amount of a combination of saxagliptin or a pharmaceutically acceptable salt, hydrate, or hydrate of a salt, thereof, a thiazolidinedione, and optionally at least one pharmaceutically acceptable carrier. In a further aspect, the present invention provides a method of increasing the time interval between a first cardiovascular event and a second cardiovascular event in mammals, particularly humans, that have survived a first cardiovascular event comprising administering to the mammal or human, in need of such treatment, a therapeutically effective amount of a combination of saxagliptin or a pharmaceutically acceptable salt, hydrate, or hydrate of a salt, thereof, a thiazolidinedione, and optionally at least one pharmaceutically acceptable carrier. Therapeutically effective amounts of saxagliptin for combination with a thiazolidinedione for prolonging the survival time following a first cardiovascular event or for increasing the time interval between a first cardiovascular event and a second cardiovascular range from about 0.5 mgs/day to about 400 mgs/day. The preferred saxagliptin doses for combination with a thiazolidinedione are 2.5 mgs/day, 5 mgs/day, and 10 mgs/day.

In another aspect, the present invention provides a method of prolonging the survival time following a second cardiovascular event in mammals, particularly humans, comprising administering to the mammal or human, in need of such treatment, a therapeutically effective amount of a combination of saxagliptin or a pharmaceutically acceptable salt, hydrate, or hydrate of a salt, thereof, a thiazolidinedione, and optionally at least one pharmaceutically acceptable carrier. In a further aspect, the present invention provides a method of increasing the time interval between a second cardiovascular event and a third cardiovascular event in mammals, particularly humans, that have survived a second cardiovascular event comprising administering to the mammal or human, in need of such treatment, a therapeutically effective amount of a combination of saxagliptin or a pharmaceutically acceptable salt, hydrate, or hydrate of a salt, thereof, a thiazolidinedione, and optionally at least one pharmaceutically acceptable carrier. Therapeutically effective amounts of saxagliptin for combination with a thiazolidinedione for prolonging the survival time following a second cardiovascular event or for increasing the time interval between a second cardiovascular event and a third cardiovascular range from about 0.5 mgs/day to about 400 mgs/day. The preferred saxagliptin doses for combination with a thiazolidinedione are 2.5 mgs/day, 5 mgs/day, and 10 mgs/day.

In another aspect, the present invention relates to methods of preventing or reducing the risk of all cause mortality in mammals, particularly humans, comprising administering to the mammal or human, in need of such treatment, a therapeutically effective amount of a combination of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier. Therapeutically effective amounts of saxagliptin for combination with rosiglitazone or pioglitazone range from about 0.5 mgs/day to about 400 mgs/day. The preferred saxagliptin doses for combination with rosiglitazone or pioglitazone are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of saxagliptin and rosiglitazone or the combination of saxagliptin and pioglitazone prevent or reduce risk of all cause mortality in T2DM patients.

In another aspect, the present invention relates to methods of preventing or reducing the risk of all cause mortality in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier. Therapeutically effective amounts of saxagliptin for combination with rosiglitazone or pioglitazone range from about 0.5 mgs/day to about 400 mgs/day. The preferred saxagliptin doses for combination with rosiglitazone or pioglitazone are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of saxagliptin and rosiglitazone or the combination of saxagliptin and pioglitazone prevent or reduce risk of all cause mortality in T2DM patients that have a history of CV disease, a history of hypertension, a history of hypercholesterolemia, and/or a smoking history (current/previous).

In another aspect, the present invention relates to methods of preventing or reducing the risk of mortality caused by a cardiovascular event in mammals, particularly humans, comprising administering to the mammal or human, in need of such treatment, a therapeutically effective amount of a combination of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier. Therapeutically effective amounts of saxagliptin for combination with rosiglitazone or pioglitazone range from about 0.5 mgs/day to about 400 mgs/day. The preferred saxagliptin doses for combination with rosiglitazone or pioglitazone are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of saxagliptin and rosiglitazone or the combination of saxagliptin and pioglitazone prevent or reduce risk of mortality caused by a CV event in T2DM patients.

In another aspect, the present invention relates to methods of preventing or reducing the risk of mortality caused by a cardiovascular event in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier. Therapeutically effective amounts of saxagliptin for combination with rosiglitazone or pioglitazone range from about 0.5 mgs/day to about 400 mgs/day. The preferred saxagliptin doses for combination with rosiglitazone or pioglitazone are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of saxagliptin and rosiglitazone or the combination of saxagliptin and pioglitazone prevent or reduce risk of mortality caused by a cardiovascular event in T2DM patients that have a history of CV disease, a history of hypertension, a history of hypercholesterolemia, and/or a smoking history (current/previous).

In another aspect, the present invention relates to methods of preventing or reducing the risk of non-fatal myocardial infarction and/or non-fatal stroke in mammals, particularly humans, comprising administering to the mammal or human, in need of such treatment, a therapeutically effective amount of a combination of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier. Therapeutically effective amounts of saxagliptin for combination with rosiglitazone or pioglitazone range from about 0.5 mgs/day to about 400 mgs/day. The preferred saxagliptin doses for combination with rosiglitazone or pioglitazone are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of saxagliptin and rosiglitazone or the combination of saxagliptin and pioglitazone prevent or reduce risk of non-fatal myocardial infarction and/or non-fatal stroke in T2DM patients.

In another aspect, the present invention relates to methods of preventing or reducing the risk of non-fatal myocardial infarction and/or non-fatal stroke in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier. Therapeutically effective amounts of saxagliptin for combination with rosiglitazone or pioglitazone range from about 0.5 mgs/day to about 400 mgs/day. The preferred saxagliptin doses for combination with rosiglitazone or pioglitazone are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of saxagliptin and rosiglitazone or the combination of saxagliptin and pioglitazone prevent or reduce risk of non-fatal myocardial infarction and/or non-fatal stroke in T2DM patients that have a history of cardiovascular disease, a history of hypertension, a history of hypercholesterolemia, and/or a smoking history (current/previous).

In another aspect, the present invention relates to methods of preventing or reducing the risk of mortality caused by a second cardiovascular event in mammals, particularly humans, that have survived a first cardiovascular event comprising administering to the mammal or human, in need of such treatment, a therapeutically effective amount of a combination of saxagliptin or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier. Therapeutically effective amounts of saxagliptin for combination with rosiglitazone or pioglitazone for preventing or reducing the risk of mortality caused by a second cardiovascular event range from about 0.5 mgs/day to about 400 mgs/day. The preferred saxagliptin doses for combination with rosiglitazone or pioglitazone are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of saxagliptin and rosiglitazone or the combination of saxagliptin and pioglitazone prevent or reduce the risk of mortality caused by a second cardiovascular event in T2DM patients that have survived a first cardiovascular event.

In another aspect, the present invention relates to methods of preventing or reducing the risk of mortality caused by a third cardiovascular event in mammals, particularly humans, that have survived two previous cardiovascular events comprising administering to the mammal or human, in need of such treatment, a therapeutically effective amount of a combination of saxagliptin or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier. Therapeutically effective amounts of saxagliptin for combination with rosiglitazone or pioglitazone for preventing or reducing the risk of mortality caused by a third cardiovascular event range from about 0.5 mgs/day to about 400 mgs/day. The preferred saxagliptin doses for combination with rosiglitazone or pioglitazone are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of saxagliptin and rosiglitazone or the combination of saxagliptin and pioglitazone prevent or reduce the risk of mortality caused by a third cardiovascular event in T2DM patients that have survived a first cardiovascular event.

In another aspect, the present invention provides a method of prolonging the survival time following a first cardiovascular event in mammals, particularly humans, comprising administering to the mammal or human, in need of such treatment, a therapeutically effective amount of a combination of saxagliptin or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier. In a further aspect, the present invention provides a method of increasing the time interval between a first cardiovascular event and a second cardiovascular event in mammals, particularly humans, that have survived a first cardiovascular event comprising administering to the mammal or human, in need of such treatment, a therapeutically effective amount of a combination of saxagliptin or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier. Therapeutically effective amounts of saxagliptin for combination with rosiglitazone or pioglitazone range from about 0.5 mgs/day to about 400 mgs/day. The preferred saxagliptin doses for combination with rosiglitazone or pioglitazone are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl.

In another aspect, the present invention provides a method of prolonging the survival time following a second cardiovascular event in mammals, particularly humans, comprising administering to the mammal or human, in need of such treatment, a therapeutically effective amount of a combination of saxagliptin or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier. In a further aspect, the present invention provides a method of increasing the time interval between a second cardiovascular event and a third cardiovascular event in mammals, particularly humans, that have survived a second cardiovascular event comprising administering to the mammal or human, in need of such treatment, a therapeutically effective amount of a combination of saxagliptin or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier. Therapeutically effective amounts of saxagliptin for combination with rosiglitazone or pioglitazone range from about 0.5 mgs/day to about 400 mgs/day. The preferred saxagliptin doses for combination with rosiglitazone or pioglitazone are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl.

In another aspect, the present invention relates to methods of preventing or reducing the risk of all cause mortality in mammals, particularly humans, comprising administering to the mammal or human, in need of such treatment, a therapeutically effective amount of alogliptin, sitagliptin, vildagliptin, or BI 1356, or a pharmaceutically acceptable salt thereof, and optionally at least one pharmaceutically acceptable carrier. Therapeutically effective amounts of alogliptin, sitagliptin, vildagliptin, or BI 1356 for preventing or reducing the risk of all cause mortality range from about 0.5 mgs/day to about 400 mgs/day. The preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of alogliptin, sitagliptin, vildagliptin, or BI 1356 prevent or reduce risk of all cause mortality in T2DM patients.

In another aspect, the present invention relates to methods of preventing or reducing the risk of all cause mortality in mammals, particularly humans, that have a history of CV disease, a history of hypertension, a history of hypercholesterolemia, and/or a smoking history (current/previous), comprising administering to the mammal or human, in need of such treatment, a therapeutically effective amount of alogliptin, sitagliptin, vildagliptin, or BI 1356, or a pharmaceutically acceptable salt thereof, and optionally at least one pharmaceutically acceptable carrier. Therapeutically effective amounts of alogliptin, sitagliptin, vildagliptin, or BI 1356 for preventing or reducing the risk of all cause mortality range from about 0.5 mgs/day to about 400 mgs/day. The preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of alogliptin, sitagliptin, vildagliptin, or BI 1356 prevent or reduce risk of all cause mortality in T2DM patients that have a history of CV disease, a history of hypertension, a history of hypercholesterolemia, and/or a smoking history (current/previous).

In another aspect, the present invention relates to methods of preventing or reducing the risk of mortality caused by a cardiovascular event in mammals, particularly humans, comprising administering to the mammal or human, in need of such treatment, a therapeutically effective amount of alogliptin, sitagliptin, vildagliptin, or BI 1356, or a pharmaceutically acceptable salt thereof, and optionally at least one pharmaceutically acceptable carrier. Therapeutically effective amounts of alogliptin, sitagliptin, vildagliptin, or BI 1356 for preventing or reducing the risk of mortality caused by a CV event range from about 0.5 mgs/day to about 400 mgs/day. The preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of alogliptin, sitagliptin, vildagliptin, or BI 1356 prevent or reduce risk of mortality caused by a CV event in T2DM patients.

In another aspect, the present invention relates to methods of preventing or reducing the risk of mortality caused by a cardiovascular event in mammals, particularly humans, that have a history of CV disease comprising administering to the mammal or human, in need of such treatment, a therapeutically effective amount of a alogliptin, sitagliptin, vildagliptin, or BI 1356 or a pharmaceutically acceptable salt thereof, and optionally at least one pharmaceutically acceptable carrier. Therapeutically effective amounts of alogliptin, sitagliptin, vildagliptin, or BI 1356 for preventing or reducing the risk of mortality caused by a CV event range from about 0.5 mgs/day to about 400 mgs/day. The preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of alogliptin, sitagliptin, vildagliptin, or BI 1356 prevent or reduce the risk of mortality caused by a CV event in T2DM patients that have a history of CV disease.

In another aspect, the present invention relates to methods of preventing or reducing the risk of mortality caused by a cardiovascular event in mammals, particularly humans, that have a history of hypertension comprising administering to the mammal or human, in need of such treatment, a therapeutically effective amount of a alogliptin, sitagliptin, vildagliptin, or BI 1356 or a pharmaceutically acceptable salt thereof, and optionally at least one pharmaceutically acceptable carrier. Therapeutically effective amounts of alogliptin, sitagliptin, vildagliptin, or BI 1356 for preventing or reducing the risk of mortality caused by a CV event range from about 0.5 mgs/day to about 400 mgs/day. The preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of alogliptin, sitagliptin, vildagliptin, or BI 1356 prevent or reduce the risk of mortality caused by a CV event in T2DM patients that have a history of hypertension.

In another aspect, the present invention relates to methods of preventing or reducing the risk of mortality caused by a cardiovascular event in mammals, particularly humans, that have a history of hypercholesterolemia comprising administering to the mammal or human, in need of such treatment, a therapeutically effective amount of a alogliptin, sitagliptin, vildagliptin, or BI 1356 or a pharmaceutically acceptable salt thereof, and optionally at least one pharmaceutically acceptable carrier. Therapeutically effective amounts of alogliptin, sitagliptin, vildagliptin, or BI 1356 for preventing or reducing the risk of mortality caused by a CV event range from about 0.5 mgs/day to about 400 mgs/day. The preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of alogliptin, sitagliptin, vildagliptin, or BI 1356 prevent or reduce the risk of mortality caused by a CV event in T2DM patients that have a history of hypercholesterolemia.

In another aspect, the present invention relates to methods of preventing or reducing the risk of mortality caused by a cardiovascular event in mammals, particularly humans, that have a current or previous smoking history comprising administering to the mammal or human, in need of such treatment, a therapeutically effective amount of a alogliptin, sitagliptin, vildagliptin, or BI 1356 or a pharmaceutically acceptable salt thereof, and optionally at least one pharmaceutically acceptable carrier. Therapeutically effective amounts of alogliptin, sitagliptin, vildagliptin, or BI 1356 for preventing or reducing the risk of mortality caused by a CV event range from about 0.5 mgs/day to about 400 mgs/day. The preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of alogliptin, sitagliptin, vildagliptin, or BI 1356 prevent or reduce the risk of mortality caused by a CV event in T2DM patients that have a current or previous smoking history.

In another aspect, the present invention relates to methods of preventing or reducing the risk of non-fatal myocardial infarction and/or non-fatal stroke in mammals, particularly humans, comprising administering to the mammal or human, in need of such treatment, a therapeutically effective amount of alogliptin, sitagliptin, vildagliptin, or BI 1356 or a pharmaceutically acceptable salt thereof, and optionally at least one pharmaceutically acceptable carrier. Therapeutically effective amounts of alogliptin, sitagliptin, vildagliptin, or BI 1356 for preventing or reducing the risk of non-fatal myocardial infarction and/or non-fatal stroke range from about 0.5 mgs/day to about 400 mgs/day. The preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of alogliptin, sitagliptin, vildagliptin, or BI 1356 prevent or reduce non-fatal myocardial infarction and/or non-fatal stroke in T2DM patients.

In another aspect, the present invention relates to methods of preventing or reducing the risk of non-fatal myocardial infarction and/or non-fatal stroke in mammals, particularly humans, that have a history of CV disease, a history of hypertension, a history of hypercholesterolemia, and/or a smoking history (current/previous), comprising administering to the mammal or human, in need of such treatment, a therapeutically effective amount of alogliptin, sitagliptin, vildagliptin, or BI 1356 or a pharmaceutically acceptable salt thereof, and optionally at least one pharmaceutically acceptable carrier. Therapeutically effective amounts of alogliptin, sitagliptin, vildagliptin, or BI 1356 for preventing or reducing the risk of non-fatal myocardial infarction and/or non-fatal stroke range from about 0.5 mgs/day to about 400 mgs/day. The preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of alogliptin, sitagliptin, vildagliptin, or BI 1356 prevent or reduce non-fatal myocardial infarction and/or non-fatal stroke in T2DM patients that have a history of CV disease, a history of hypertension, a history of hypercholesterolemia, and/or a smoking history (current/previous).

In another aspect, the present invention relates to methods of preventing or reducing the risk of mortality caused by a second cardiovascular event in mammals, particularly humans, that have survived a first cardiovascular event comprising administering to the mammal or human, in need of such treatment, a therapeutically effective amount of a alogliptin, sitagliptin, vildagliptin, or BI 1356 or a pharmaceutically acceptable salt thereof, and optionally at least one pharmaceutically acceptable carrier. Therapeutically effective amounts of alogliptin, sitagliptin, vildagliptin, or BI 1356 for preventing or reducing the risk of mortality caused by a second CV event range from about 0.5 mgs/day to about 400 mgs/day. The preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of alogliptin, sitagliptin, vildagliptin, or BI 1356 prevent or reduce the risk of mortality caused by a second CV event in T2DM patients that have survived a first CV event.

In another aspect, the present invention relates to methods of preventing or reducing the risk of mortality caused by a third cardiovascular event in mammals, particularly humans, that have survived two cardiovascular events comprising administering to the mammal or human, in need of such treatment, a therapeutically effective amount of a alogliptin, sitagliptin, vildagliptin, or BI 1356 or a pharmaceutically acceptable salt thereof, and optionally at least one pharmaceutically acceptable carrier. Therapeutically effective amounts of alogliptin, sitagliptin, vildagliptin, or BI 1356 for preventing or reducing the risk of mortality caused by a third CV event range from about 0.5 mgs/day to about 400 mgs/day. The preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of alogliptin, sitagliptin, vildagliptin, or BI 1356 prevent or reduce the risk of mortality caused by a third CV event in T2DM patients that have survived two cardiovascular events.

In another aspect, the present invention provides a method of prolonging the survival time following a first cardiovascular event in mammals, particularly humans, comprising administering to the mammal or human, in need of such treatment, a therapeutically effective amount of alogliptin, sitagliptin, vildagliptin, or BI 1356, or a pharmaceutically acceptable salt thereof, and optionally at least one pharmaceutically acceptable carrier. In a further aspect, the present invention provides a method of increasing the time interval between a first cardiovascular event and a second cardiovascular event in mammals, particularly humans, that have survived a first cardiovascular event comprising administering to the mammal or human, in need of such treatment, a therapeutically effective amount of alogliptin, sitagliptin, vildagliptin, or BI 1356, or a pharmaceutically acceptable salt thereof, and optionally at least one pharmaceutically acceptable carrier. Therapeutically effective amounts of alogliptin, sitagliptin, vildagliptin, or BI 1356 for prolonging the survival time following a first cardiovascular event or for increasing the time interval between a first cardiovascular event and a second cardiovascular range from about 0.5 mgs/day to about 400 mgs/day. The preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention provides a method of prolonging the survival time following a second cardiovascular event in mammals, particularly humans, comprising administering to the mammal or human, in need of such treatment, a therapeutically effective amount of alogliptin, sitagliptin, vildagliptin, or BI 1356, or a pharmaceutically acceptable salt thereof, and optionally at least one pharmaceutically acceptable carrier. In a further aspect, the present invention provides a method of increasing the time interval between a second cardiovascular event and a third cardiovascular event in mammals, particularly humans, that have survived a second cardiovascular event comprising administering to the mammal or human, in need of such treatment, a therapeutically effective amount of alogliptin, sitagliptin, vildagliptin, or BI 1356, or a pharmaceutically acceptable salt thereof, and optionally at least one pharmaceutically acceptable carrier. Therapeutically effective amounts of alogliptin, sitagliptin, vildagliptin, or BI 1356 for prolonging the survival time following a second cardiovascular event or for increasing the time interval between a second cardiovascular event and a third cardiovascular range from about 0.5 mgs/day to about 400 mgs/day. The preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention relates to methods of preventing or reducing the risk of all cause mortality in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, a sulfonylurea, and optionally at least one pharmaceutically acceptable carrier. Therapeutically effective amounts of alogliptin, sitagliptin, vildagliptin, or BI 1356 for combination with a sulfonylurea range from about 0.5 mgs/day to about 400 mgs/day. The preferred alogliptin, sitagliptin, vildagliptin, or BI 1356 doses for combination with a sulfonylurea are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a sulfonylurea prevent or reduce risk of all cause mortality in T2DM patients.

In another aspect, the present invention relates to methods of preventing or reducing the risk of all cause mortality in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, a sulfonylurea, and optionally at least one pharmaceutically acceptable carrier. Therapeutically effective amounts of alogliptin, sitagliptin, vildagliptin, or BI 1356 for combination with a sulfonylurea range from about 0.5 mgs/day to about 400 mgs/day. The preferred alogliptin, sitagliptin, vildagliptin, or BI 1356 doses for combination with a sulfonylurea are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a sulfonylurea prevent or reduce risk of all cause mortality in T2DM patients that have a history of CV disease, a history of hypertension, a history of hypercholesterolemia, and/or a smoking history (current/previous).

In another aspect, the present invention relates to methods of preventing or reducing the risk of mortality caused by a cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, a sulfonylurea, and optionally at least one pharmaceutically acceptable carrier. Therapeutically effective amounts of alogliptin, sitagliptin, vildagliptin, or BI 1356 for combination with a sulfonylurea range from about 0.5 mgs/day to about 400 mgs/day. The preferred alogliptin, sitagliptin, vildagliptin, or BI 1356 doses for combination with a sulfonylurea are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a sulfonylurea prevent or reduce risk of mortality caused by a CV event in T2DM patients.

In another aspect, the present invention relates to methods of preventing or reducing the risk of mortality caused by a cardiovascular event in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, a sulfonylurea, and optionally at least one pharmaceutically acceptable carrier. Therapeutically effective amounts of alogliptin, sitagliptin, vildagliptin, or BI 1356 for combination with a sulfonylurea range from about 0.5 mgs/day to about 400 mgs/day. The preferred alogliptin, sitagliptin, vildagliptin, or BI 1356 doses for combination with a sulfonylurea are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a sulfonylurea prevent or reduce risk of mortality caused by a CV event in T2DM patients that have a history of CV disease, a history of hypertension, a history of hypercholesterolemia, and/or a smoking history (current/previous).

In another aspect, the present invention relates to methods of preventing or reducing the risk of non-fatal myocardial infarction and/or non-fatal stroke in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, a sulfonylurea, and optionally at least one pharmaceutically acceptable carrier. Therapeutically effective amounts of alogliptin, sitagliptin, vildagliptin, or BI 1356 for combination with a sulfonylurea range from about 0.5 mgs/day to about 400 mgs/day. The preferred alogliptin, sitagliptin, vildagliptin, or BI 1356 doses for combination with a sulfonylurea are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a sulfonylurea prevent or reduce risk of non-fatal myocardial infarction and/or non-fatal stroke in T2DM patients.

In another aspect, the present invention relates to methods of preventing or reducing the risk of non-fatal myocardial infarction and/or non-fatal stroke in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, a sulfonylurea, and optionally at least one pharmaceutically acceptable carrier. Therapeutically effective amounts of alogliptin, sitagliptin, vildagliptin, or BI 1356 for combination with a sulfonylurea range from about 0.5 mgs/day to about 400 mgs/day. The preferred alogliptin, sitagliptin, vildagliptin, or BI 1356 doses for combination with a sulfonylurea are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a sulfonylurea prevent or reduce risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to methods of preventing or reducing the risk of mortality caused by a second cardiovascular event in mammals, particularly humans, that have survived a first cardiovascular event comprising administering to the mammal or human, in need of such treatment, a therapeutically effective amount of a combination of alogliptin, sitagliptin, vildagliptin, or BI 1356, or a pharmaceutically acceptable salt thereof, a sulfonylurea, and optionally at least one pharmaceutically acceptable carrier. Therapeutically effective amounts of alogliptin, sitagliptin, vildagliptin, or BI 1356 for combination with a sulfonylurea for preventing or reducing the risk of mortality caused by a second CV event range from about 0.5 mgs/day to about 400 mgs/day. The preferred alogliptin, sitagliptin, vildagliptin, or BI 1356 doses for combination with a sulfonylurea are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a sulfonylurea prevent or reduce the risk of mortality caused by a second CV event in T2DM patients.

In another aspect, the present invention relates to methods of preventing or reducing the risk of mortality caused by a third cardiovascular event in mammals, particularly humans, that have survived two previous cardiovascular events comprising administering to the mammal or human, in need of such treatment, a therapeutically effective amount of a combination of alogliptin, sitagliptin, vildagliptin, or BI 1356, or a pharmaceutically acceptable salt thereof, a sulfonylurea, and optionally at least one pharmaceutically acceptable carrier. Therapeutically effective amounts of alogliptin, sitagliptin, vildagliptin, or BI 1356 for combination with a sulfonylurea for preventing or reducing the risk of mortality caused by a third CV event range from about 0.5 mgs/day to about 400 mgs/day. The preferred alogliptin, sitagliptin, vildagliptin, or BI 1356 doses for combination with a sulfonylurea are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a sulfonylurea prevent or reduce the risk of mortality caused by a third CV event in T2DM patients.

In another aspect, the present invention provides a method of prolonging the survival time following a first cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, a sulfonylurea, and optionally at least one pharmaceutically acceptable carrier. In a further aspect, the present invention provides a method of increasing the time interval between a first cardiovascular event and a second cardiovascular event in mammals, particularly humans, that have survived a first cardiovascular event comprising administering to the mammal or human, in need of such treatment, a therapeutically effective amount of a combination of alogliptin, sitagliptin, vildagliptin, or BI 1356, or a pharmaceutically acceptable salt thereof, a sulfonylurea, and optionally at least one pharmaceutically acceptable carrier. Therapeutically effective amounts of alogliptin, sitagliptin, vildagliptin, or BI 1356 for combination with a sulfonylurea for prolonging the survival time following a first cardiovascular event or for increasing the time interval between a first cardiovascular event and a second cardiovascular range from about 0.5 mgs/day to about 400 mgs/day. The preferred alogliptin, sitagliptin, vildagliptin, or BI 1356 doses for combination with a sulfonylurea are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention provides a method of prolonging the survival time following a second cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, a sulfonylurea, and optionally at least one pharmaceutically acceptable carrier. In a further aspect, the present invention provides a method of increasing the time interval between a second cardiovascular event and a third cardiovascular event in mammals, particularly humans, that have survived a second cardiovascular event comprising administering to the mammal or human, in need of such treatment, a therapeutically effective amount of a combination of alogliptin, sitagliptin, vildagliptin, or BI 1356, or a pharmaceutically acceptable salt thereof, a sulfonylurea, and optionally at least one pharmaceutically acceptable carrier. Therapeutically effective amounts of alogliptin, sitagliptin, vildagliptin, or BI 1356 for combination with a sulfonylurea for prolonging the survival time following a second cardiovascular event or for increasing the time interval between a second cardiovascular event and a third cardiovascular range from about 0.5 mgs/day to about 400 mgs/day. The preferred alogliptin, sitagliptin, vildagliptin, or BI 1356 doses for combination with a sulfonylurea are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention relates to methods of preventing or reducing the risk of all cause mortality in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, glyburide, and optionally at least one pharmaceutically acceptable carrier. Therapeutically effective amounts of alogliptin, sitagliptin, vildagliptin, or BI 1356 for combination with glyburide range from about 0.5 mgs/day to about 400 mgs/day. The preferred alogliptin, sitagliptin, vildagliptin, or BI 1356 doses for combination with glyburide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Therapeutically effective amounts of the combination of alogliptin, sitagliptin, vildagliptin, or BI 1356 and glyburide prevent or reduce risk of all cause mortality in T2DM patients.

In another aspect, the present invention relates to methods of preventing or reducing the risk of all cause mortality in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, glyburide, and optionally at least one pharmaceutically acceptable carrier. Therapeutically effective amounts of alogliptin, sitagliptin, vildagliptin, or BI 1356 for combination with glyburide range from about 0.5 mgs/day to about 400 mgs/day. The preferred alogliptin, sitagliptin, vildagliptin, or BI 1356 doses for combination with glyburide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Therapeutically effective amounts of the combination of alogliptin, sitagliptin, vildagliptin, or BI 1356 and glyburide prevent or reduce risk of all cause mortality in T2DM patients that have a history of CV disease, a history of hypertension, a history of hypercholesterolemia, and/or a smoking history (current/previous).

In another aspect, the present invention relates to methods of preventing or reducing the risk of mortality caused by a cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, glyburide, and optionally at least one pharmaceutically acceptable carrier. Therapeutically effective amounts of alogliptin, sitagliptin, vildagliptin, or BI 1356 for combination with glyburide range from about 0.5 mgs/day to about 400 mgs/day. The preferred alogliptin, sitagliptin, vildagliptin, or BI 1356 doses for combination with glyburide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Therapeutically effective amounts of the combination of alogliptin, sitagliptin, vildagliptin, or BI 1356 and glyburide prevent or reduce risk of mortality caused by a CV event in T2DM patients.

In another aspect, the present invention relates to methods of preventing or reducing the risk of mortality caused by a cardiovascular event in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, glyburide, and optionally at least one pharmaceutically acceptable carrier. Therapeutically effective amounts of alogliptin, sitagliptin, vildagliptin, or BI 1356 for combination with glyburide range from about 0.5 mgs/day to about 400 mgs/day. The preferred alogliptin, sitagliptin, vildagliptin, or BI 1356 doses for combination with glyburide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Therapeutically effective amounts of the combination of alogliptin, sitagliptin, vildagliptin, or BI 1356 and glyburide prevent or reduce risk of mortality caused by a CV event in T2DM patients that have a history of CV disease, a history of hypertension, a history of hypercholesterolemia, and/or a smoking history (current/previous).

In another aspect, the present invention relates to methods of preventing or reducing the risk of non-fatal myocardial infarction and/or non-fatal stroke in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, glyburide, and optionally at least one pharmaceutically acceptable carrier. Therapeutically effective amounts of alogliptin, sitagliptin, vildagliptin, or BI 1356 for combination with glyburide range from about 0.5 mgs/day to about 400 mgs/day. The preferred alogliptin, sitagliptin, vildagliptin, or BI 1356 doses for combination with glyburide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Therapeutically effective amounts of the combination of alogliptin, sitagliptin, vildagliptin, or BI 1356 and glyburide prevent or reduce risk of non-fatal myocardial infarction and/or non-fatal stroke in T2DM patients.

In another aspect, the present invention relates to methods of preventing or reducing the risk of non-fatal myocardial infarction and/or non-fatal stroke in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, glyburide, and optionally at least one pharmaceutically acceptable carrier. Therapeutically effective amounts of alogliptin, sitagliptin, vildagliptin, or BI 1356 for combination with glyburide range from about 0.5 mgs/day to about 400 mgs/day. The preferred alogliptin, sitagliptin, vildagliptin, or BI 1356 doses for combination with glyburide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Therapeutically effective amounts of the combination of alogliptin, sitagliptin, vildagliptin, or BI 1356 and glyburide prevent or reduce risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to methods of preventing or reducing the risk of mortality caused by a second cardiovascular event in mammals, particularly humans, that have survived a first cardiovascular event comprising administering to the mammal or human, in need of such treatment, a therapeutically effective amount of a combination of alogliptin, sitagliptin, vildagliptin, or BI 1356, or a pharmaceutically acceptable salt thereof, glyburide, and optionally at least one pharmaceutically acceptable carrier. Therapeutically effective amounts of alogliptin, sitagliptin, vildagliptin, or BI 1356 for combination with glyburide for preventing or reducing the risk of mortality caused by a second CV event range from about 0.5 mgs/day to about 400 mgs/day. The preferred alogliptin, sitagliptin, vildagliptin, or BI 1356 doses for combination with glyburide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Therapeutically effective amounts of the combination of alogliptin, sitagliptin, vildagliptin, or BI 1356 and glyburide prevent or reduce the risk of mortality caused by a second CV event in T2DM patients.

In another aspect, the present invention relates to methods of preventing or reducing the risk of mortality caused by a third cardiovascular event in mammals, particularly humans, that have survived two previous cardiovascular events comprising administering to the mammal or human, in need of such treatment, a therapeutically effective amount of a combination of alogliptin, sitagliptin, vildagliptin, or BI 1356, or a pharmaceutically acceptable salt thereof, glyburide, and optionally at least one pharmaceutically acceptable carrier. Therapeutically effective amounts of alogliptin, sitagliptin, vildagliptin, or BI 1356 for combination with glyburide for preventing or reducing the risk of mortality caused by a second CV event range from about 0.5 mgs/day to about 400 mgs/day. The preferred alogliptin, sitagliptin, vildagliptin, or BI 1356 doses for combination with glyburide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Therapeutically effective amounts of the combination of alogliptin, sitagliptin, vildagliptin, or BI 1356 and glyburide prevent or reduce the risk of mortality caused by a third CV event in T2DM patients.

In another aspect, the present invention provides a method of prolonging the survival time following a first cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, glyburide, and optionally at least one pharmaceutically acceptable carrier. In a further aspect, the present invention provides a method of increasing the time interval between a first cardiovascular event and a second cardiovascular event in mammals, particularly humans, that have survived a first cardiovascular event comprising administering to the mammal or human, in need of such treatment, a therapeutically effective amount of a combination of alogliptin, sitagliptin, vildagliptin, or BI 1356, or a pharmaceutically acceptable salt thereof, glyburide, and optionally at least one pharmaceutically acceptable carrier. Therapeutically effective amounts of alogliptin, sitagliptin, vildagliptin, or BI 1356 for combination with glyburide for prolonging the survival time following a first cardiovascular event or for increasing the time interval between a first cardiovascular event and a second cardiovascular range from about 0.5 mgs/day to about 400 mgs/day. The preferred alogliptin, sitagliptin, vildagliptin, or BI 1356 doses for combination with glyburide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day.

In another aspect, the present invention provides a method of prolonging the survival time following a second cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, glyburide, and optionally at least one pharmaceutically acceptable carrier. In a further aspect, the present invention provides a method of increasing the time interval between a second cardiovascular event and a third cardiovascular event in mammals, particularly humans, that have survived a second cardiovascular event comprising administering to the mammal or human, in need of such treatment, a therapeutically effective amount of a combination of alogliptin, sitagliptin, vildagliptin, or BI 1356, or a pharmaceutically acceptable salt thereof, glyburide, and optionally at least one pharmaceutically acceptable carrier. Therapeutically effective amounts of alogliptin, sitagliptin, vildagliptin, or BI 1356 for combination with glyburide for prolonging the survival time following a second cardiovascular event or for increasing the time interval between a second cardiovascular event and a third cardiovascular range from about 0.5 mgs/day to about 400 mgs/day. The preferred alogliptin, sitagliptin, vildagliptin, or BI 1356 doses for combination with glyburide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day.

In another aspect, the present invention relates to methods of preventing or reducing the risk of all cause mortality in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, a biguanide, and optionally at least one pharmaceutically acceptable carrier. Therapeutically effective amounts of alogliptin, sitagliptin, vildagliptin, or BI 1356 for combination with a biguanide range from about 0.5 mgs/day to about 400 mgs/day. The preferred alogliptin, sitagliptin, vildagliptin, or BI 1356 doses for combination with a biguanide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a biguanide prevent or reduce risk of all cause mortality in T2DM patients.

In another aspect, the present invention relates to methods of preventing or reducing the risk of all cause mortality in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, a biguanide, and optionally at least one pharmaceutically acceptable carrier. Therapeutically effective amounts of alogliptin, sitagliptin, vildagliptin, or BI 1356 for combination with a biguanide range from about 0.5 mgs/day to about 400 mgs/day. The preferred alogliptin, sitagliptin, vildagliptin, or BI 1356 doses for combination with a biguanide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a biguanide prevent or reduce risk of all cause mortality in T2DM patients that have a history of CV disease, a history of hypertension, a history of hypercholesterolemia, and/or a smoking history (current/previous).

In another aspect, the present invention relates to methods of preventing or reducing the risk of mortality caused by a cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, a biguanide, and optionally at least one pharmaceutically acceptable carrier. Therapeutically effective amounts of alogliptin, sitagliptin, vildagliptin, or BI 1356 for combination with a biguanide range from about 0.5 mgs/day to about 400 mgs/day. The preferred alogliptin, sitagliptin, vildagliptin, or BI 1356 doses for combination with a biguanide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a biguanide prevent or reduce risk of mortality caused by a CV event in T2DM patients.

In another aspect, the present invention relates to methods of preventing or reducing the risk of mortality caused by a cardiovascular event in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, a biguanide, and optionally at least one pharmaceutically acceptable carrier. Therapeutically effective amounts of alogliptin, sitagliptin, vildagliptin, or BI 1356 for combination with a biguanide range from about 0.5 mgs/day to about 400 mgs/day. The preferred alogliptin, sitagliptin, vildagliptin, or BI 1356 doses for combination with a biguanide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a biguanide prevent or reduce risk of mortality caused by a CV event in T2DM patients that have a history of CV disease, a history of hypertension, a history of hypercholesterolemia, and/or a smoking history (current/previous).

In another aspect, the present invention relates to methods of preventing or reducing the risk of non-fatal myocardial infarction and/or non-fatal stroke in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, a biguanide, and optionally at least one pharmaceutically acceptable carrier. Therapeutically effective amounts of alogliptin, sitagliptin, vildagliptin, or BI 1356 for combination with a biguanide range from about 0.5 mgs/day to about 400 mgs/day. The preferred alogliptin, sitagliptin, vildagliptin, or BI 1356 doses for combination with a biguanide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a biguanide prevent or reduce risk of non-fatal myocardial infarction and/or non-fatal stroke in T2DM patients.

In another aspect, the present invention relates to methods of preventing or reducing the risk of non-fatal myocardial infarction and/or non-fatal stroke in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, a biguanide, and optionally at least one pharmaceutically acceptable carrier. Therapeutically effective amounts of alogliptin, sitagliptin, vildagliptin, or BI 1356 for combination with a biguanide range from about 0.5 mgs/day to about 400 mgs/day. The preferred alogliptin, sitagliptin, vildagliptin, or BI 1356 doses for combination with a biguanide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a biguanide prevent or reduce risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to methods of preventing or reducing the risk of mortality caused by a second cardiovascular event in mammals, particularly humans, that have survived a first cardiovascular event comprising administering to the mammal or human, in need of such treatment, a therapeutically effective amount of a combination of alogliptin, sitagliptin, vildagliptin, or BI 1356 or a pharmaceutically acceptable salt thereof, a biguanide, and optionally at least one pharmaceutically acceptable carrier. Therapeutically effective amounts of alogliptin, sitagliptin, vildagliptin, or BI 1356 for combination with a biguanide for preventing or reducing the risk of mortality caused by a second CV event range from about 0.5 mgs/day to about 400 mgs/day. The preferred alogliptin, sitagliptin, vildagliptin, or BI 1356 doses for combination with a biguanide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a biguanide prevent or reduce the risk of mortality caused by a second CV event in T2DM patients.

In another aspect, the present invention relates to methods of preventing or reducing the risk of mortality caused by a third cardiovascular event in mammals, particularly humans, that have survived two previous cardiovascular events comprising administering to the mammal or human, in need of such treatment, a therapeutically effective amount of a combination of alogliptin, sitagliptin, vildagliptin, or BI 1356, or a pharmaceutically acceptable salt thereof, a biguanide, and optionally at least one pharmaceutically acceptable carrier. Therapeutically effective amounts of alogliptin, sitagliptin, vildagliptin, or BI 1356 for combination with a biguanide for preventing or reducing the risk of mortality caused by a third CV event range from about 0.5 mgs/day to about 400 mgs/day. The preferred alogliptin, sitagliptin, vildagliptin, or BI 1356 doses for combination with a biguanide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a biguanide prevent or reduce the risk of mortality caused by a third CV event in T2DM patients.

In another aspect, the present invention provides a method of prolonging the survival time following a first cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, a biguanide, and optionally at least one pharmaceutically acceptable carrier. In a further aspect, the present invention provides a method of increasing the time interval between a first cardiovascular event and a second cardiovascular event in mammals, particularly humans, that have survived a first cardiovascular event comprising administering to the mammal or human, in need of such treatment, a therapeutically effective amount of a combination of alogliptin, sitagliptin, vildagliptin, or BI 1356, or a pharmaceutically acceptable salt thereof, a biguanide, and optionally at least one pharmaceutically acceptable carrier. Therapeutically effective amounts of alogliptin, sitagliptin, vildagliptin, or BI 1356 for combination with a biguanide for prolonging the survival time following a first cardiovascular event or for increasing the time interval between a first cardiovascular event and a second cardiovascular range from about 0.5 mgs/day to about 400 mgs/day. The preferred alogliptin, sitagliptin, vildagliptin, or BI 1356 doses for combination with a biguanide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention provides a method of prolonging the survival time following a second cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, a biguanide, and optionally at least one pharmaceutically acceptable carrier. In a further aspect, the present invention provides a method of increasing the time interval between a second cardiovascular event and a third cardiovascular event in mammals, particularly humans, that have survived a second cardiovascular event comprising administering to the mammal or human, in need of such treatment, a therapeutically effective amount of a combination of alogliptin, sitagliptin, vildagliptin, or BI 1356, or a pharmaceutically acceptable salt thereof, a biguanide, and optionally at least one pharmaceutically acceptable carrier. Therapeutically effective amounts of alogliptin, sitagliptin, vildagliptin, or BI 1356 for combination with a biguanide for prolonging the survival time following a second cardiovascular event or for increasing the time interval between a second cardiovascular event and a third cardiovascular range from about 0.5 mgs/day to about 400 mgs/day. The preferred alogliptin, sitagliptin, vildagliptin, or BI 1356 doses for combination with a biguanide are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention relates to methods of preventing or reducing the risk of all cause mortality in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier. Therapeutically effective amounts of alogliptin, sitagliptin, vildagliptin, or BI 1356 for combination with metformin range from about 0.5 mgs/day to about 400 mgs/day. The preferred alogliptin, sitagliptin, vildagliptin, or BI 1356 doses for combination with metformin are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Therapeutically effective amounts of the combination of alogliptin, sitagliptin, vildagliptin, or BI 1356 and metformin prevent or reduce risk of all cause mortality in T2DM patients.

In another aspect, the present invention relates to methods of preventing or reducing the risk of all cause mortality in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier. Therapeutically effective amounts of alogliptin, sitagliptin, vildagliptin, or BI 1356 for combination with metformin range from about 0.5 mgs/day to about 400 mgs/day. The preferred alogliptin, sitagliptin, vildagliptin, or BI 1356 doses for combination with metformin are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Therapeutically effective amounts of the combination of alogliptin, sitagliptin, vildagliptin, or BI 1356 and metformin prevent or reduce risk of all cause mortality in T2DM patients that have a history of CV disease, a history of hypertension, a history of hypercholesterolemia, and/or a smoking history (current/previous).

In another aspect, the present invention relates to methods of preventing or reducing the risk of mortality caused by a cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier. Therapeutically effective amounts of alogliptin, sitagliptin, vildagliptin, or BI 1356 for combination with metformin range from about 0.5 mgs/day to about 400 mgs/day. The preferred alogliptin, sitagliptin, vildagliptin, or BI 1356 doses for combination with metformin are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Therapeutically effective amounts of the combination of alogliptin, sitagliptin, vildagliptin, or BI 1356 and metformin prevent or reduce risk of mortality caused by a CV event in T2DM patients.

In another aspect, the present invention relates to methods of preventing or reducing the risk of mortality caused by a cardiovascular event in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier. Therapeutically effective amounts of alogliptin, sitagliptin, vildagliptin, or BI 1356 for combination with metformin range from about 0.5 mgs/day to about 400 mgs/day. The preferred alogliptin, sitagliptin, vildagliptin, or BI 1356 doses for combination with metformin are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Therapeutically effective amounts of the combination of alogliptin, sitagliptin, vildagliptin, or BI 1356 and metformin prevent or reduce risk of mortality caused by a CV event in T2DM patients that have a history of CV disease, a history of hypertension, a history of hypercholesterolemia, and/or a smoking history (current/previous).

In another aspect, the present invention relates to methods of preventing or reducing the risk of non-fatal myocardial infarction and/or non-fatal stroke in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier. Therapeutically effective amounts of alogliptin, sitagliptin, vildagliptin, or BI 1356 for combination with metformin range from about 0.5 mgs/day to about 400 mgs/day. The preferred alogliptin, sitagliptin, vildagliptin, or BI 1356 doses for combination with metformin are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Therapeutically effective amounts of the combination of alogliptin, sitagliptin, vildagliptin, or BI 1356 and metformin prevent or reduce risk of non-fatal myocardial infarction and/or non-fatal stroke in T2DM patients.

In another aspect, the present invention relates to methods of preventing or reducing the risk of non-fatal myocardial infarction and/or non-fatal stroke in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier. Therapeutically effective amounts of alogliptin, sitagliptin, vildagliptin, or BI 1356 for combination with metformin range from about 0.5 mgs/day to about 400 mgs/day. The preferred alogliptin, sitagliptin, vildagliptin, or BI 1356 doses for combination with metformin are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Therapeutically effective amounts of the combination of alogliptin, sitagliptin, vildagliptin, or BI 1356 and metformin prevent or reduce risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to methods of preventing or reducing the risk of mortality caused by a second cardiovascular event in mammals, particularly humans, that have survived a first cardiovascular event comprising administering to the mammal or human, in need of such treatment, a therapeutically effective amount of a combination of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier. Therapeutically effective amounts of alogliptin, sitagliptin, vildagliptin, or BI 1356 for combination with metformin for preventing or reducing the risk of mortality caused by a second CV event range from about 0.5 mgs/day to about 400 mgs/day. The preferred alogliptin, sitagliptin, vildagliptin, or BI 1356 doses for combination with metformin are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Therapeutically effective amounts of the combination of alogliptin, sitagliptin, vildagliptin, or BI 1356 and metformin prevent or reduce the risk of mortality caused by a second CV event in T2DM patients.

In another aspect, the present invention relates to methods of preventing or reducing the risk of mortality caused by a third cardiovascular event in mammals, particularly humans, that have survived two previous cardiovascular events comprising administering to the mammal or human, in need of such treatment, a therapeutically effective amount of a combination of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier. Therapeutically effective amounts of alogliptin, sitagliptin, vildagliptin, or BI 1356 for combination with metformin for preventing or reducing the risk of mortality caused by a third CV event range from about 0.5 mgs/day to about 400 mgs/day. The preferred alogliptin, sitagliptin, vildagliptin, or BI 1356 doses for combination with metformin are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Therapeutically effective amounts of the combination of alogliptin, sitagliptin, vildagliptin, or BI 1356 and metformin prevent or reduce the risk of mortality caused by a third CV event in T2DM patients.

In another aspect, the present invention provides a method of prolonging the survival time following a first cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier. In a further aspect, the present invention provides a method of increasing the time interval between a first cardiovascular event and a second cardiovascular event in mammals, particularly humans, that have survived a first cardiovascular event comprising administering to the mammal or human, in need of such treatment, a therapeutically effective amount of a combination of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier. Therapeutically effective amounts of alogliptin, sitagliptin, vildagliptin, or BI 1356 for combination with metformin for prolonging the survival time following a first cardiovascular event or for increasing the time interval between a first cardiovascular event and a second cardiovascular range from about 0.5 mgs/day to about 400 mgs/day. The preferred alogliptin, sitagliptin, vildagliptin, or BI 1356 doses for combination with metformin are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl.

In another aspect, the present invention provides a method of prolonging the survival time following a second cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier. In a further aspect, the present invention provides a method of increasing the time interval between a second cardiovascular event and a third cardiovascular event in mammals, particularly humans, that have survived a second cardiovascular event comprising administering to the mammal or human, in need of such treatment, a therapeutically effective amount of a combination of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier. Therapeutically effective amounts of alogliptin, sitagliptin, vildagliptin, or BI 1356 for combination with metformin for prolonging the survival time following a second cardiovascular event or for increasing the time interval between a second cardiovascular event and a third cardiovascular range from about 0.5 mgs/day to about 400 mgs/day. The preferred alogliptin, sitagliptin, vildagliptin, or BI 1356 doses for combination with metformin are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl.

In another aspect, the present invention relates to methods of preventing or reducing the risk of all cause mortality in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, a thiazolidinedione, and optionally at least one pharmaceutically acceptable carrier. Therapeutically effective amounts of alogliptin, sitagliptin, vildagliptin, or BI 1356 for combination with a thiazolidinedione range from about 0.5 mgs/day to about 400 mgs/day. The preferred alogliptin, sitagliptin, vildagliptin, or BI 1356 doses for combination with a thiazolidinedione are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a thiazolidinedione prevent or reduce risk of all cause mortality in T2DM patients.

In another aspect, the present invention relates to methods of preventing or reducing the risk of all cause mortality in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, a thiazolidinedione, and optionally at least one pharmaceutically acceptable carrier. Therapeutically effective amounts of alogliptin, sitagliptin, vildagliptin, or BI 1356 for combination with a thiazolidinedione range from about 0.5 mgs/day to about 400 mgs/day. The preferred alogliptin, sitagliptin, vildagliptin, or BI 1356 doses for combination with a thiazolidinedione are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a thiazolidinedione prevent or reduce risk of all cause mortality in T2DM patients that have a history of CV disease, a history of hypertension, a history of hypercholesterolemia, and/or a smoking history (current/previous).

In another aspect, the present invention relates to methods of preventing or reducing the risk of mortality caused by a cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, a thiazolidinedione, and optionally at least one pharmaceutically acceptable carrier. Therapeutically effective amounts of alogliptin, sitagliptin, vildagliptin, or BI 1356 for combination with a thiazolidinedione range from about 0.5 mgs/day to about 400 mgs/day. The preferred alogliptin, sitagliptin, vildagliptin, or BI 1356 doses for combination with a thiazolidinedione are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a thiazolidinedione prevent or reduce risk of mortality caused by a CV event in T2DM patients.

In another aspect, the present invention relates to methods of preventing or reducing the risk of mortality caused by a cardiovascular event in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, a thiazolidinedione, and optionally at least one pharmaceutically acceptable carrier. Therapeutically effective amounts of alogliptin, sitagliptin, vildagliptin, or BI 1356 for combination with a thiazolidinedione range from about 0.5 mgs/day to about 400 mgs/day. The preferred alogliptin, sitagliptin, vildagliptin, or BI 1356 doses for combination with a thiazolidinedione are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a thiazolidinedione prevent or reduce risk of mortality caused by a CV event in T2DM patients that have a history of CV disease, a history of hypertension, a history of hypercholesterolemia, and/or a smoking history (current/previous).

In another aspect, the present invention relates to methods of preventing or reducing the risk of non-fatal myocardial infarction and/or non-fatal stroke in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, a thiazolidinedione, and optionally at least one pharmaceutically acceptable carrier. Therapeutically effective amounts of alogliptin, sitagliptin, vildagliptin, or BI 1356 for combination with a thiazolidinedione range from about 0.5 mgs/day to about 400 mgs/day. The preferred alogliptin, sitagliptin, vildagliptin, or BI 1356 doses for combination with a thiazolidinedione are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a thiazolidinedione prevent or reduce risk of non-fatal myocardial infarction and/or non-fatal stroke in T2DM patients.

In another aspect, the present invention relates to methods of preventing or reducing the risk of non-fatal myocardial infarction and/or non-fatal stroke in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, a thiazolidinedione, and optionally at least one pharmaceutically acceptable carrier. Therapeutically effective amounts of alogliptin, sitagliptin, vildagliptin, or BI 1356 for combination with a thiazolidinedione range from about 0.5 mgs/day to about 400 mgs/day. The preferred alogliptin, sitagliptin, vildagliptin, or BI 1356 doses for combination with a thiazolidinedione are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a thiazolidinedione prevent or reduce risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to methods of preventing or reducing the risk of mortality caused by a second cardiovascular event in mammals, particularly humans, that have survived a first cardiovascular event comprising administering to the mammal or human, in need of such treatment, a therapeutically effective amount of a combination of alogliptin, sitagliptin, vildagliptin, or BI 1356, or a pharmaceutically acceptable salt thereof, a thiazolidinedione, and optionally at least one pharmaceutically acceptable carrier. Therapeutically effective amounts of alogliptin, sitagliptin, vildagliptin, or BI 1356 for combination with a thiazolidinedione for preventing or reducing the risk of mortality caused by a second CV event range from about 0.5 mgs/day to about 400 mgs/day. The preferred alogliptin, sitagliptin, vildagliptin, or BI 1356 doses for combination with a thiazolidinedione are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a thiazolidinedione prevent or reduce the risk of mortality caused by a second CV event in T2DM patients.

In another aspect, the present invention relates to methods of preventing or reducing the risk of mortality caused by a third cardiovascular event in mammals, particularly humans, that have survived two previous cardiovascular events comprising administering to the mammal or human, in need of such treatment, a therapeutically effective amount of a combination of alogliptin, sitagliptin, vildagliptin, or BI 1356, or a pharmaceutically acceptable salt thereof, a thiazolidinedione, and optionally at least one pharmaceutically acceptable carrier. Therapeutically effective amounts of alogliptin, sitagliptin, vildagliptin, or BI 1356 for combination with a thiazolidinedione for preventing or reducing the risk of mortality caused by a third CV event range from about 0.5 mgs/day to about 400 mgs/day. The preferred alogliptin, sitagliptin, vildagliptin, or BI 1356 doses for combination with a thiazolidinedione are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Therapeutically effective amounts of the combination of alogliptin, sitagliptin, vildagliptin, or BI 1356 and a thiazolidinedione prevent or reduce the risk of mortality caused by a third CV event in T2DM patients.

In another aspect, the present invention provides a method of prolonging the survival time following a first cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, a thiazolidinedione, and optionally at least one pharmaceutically acceptable carrier. In a further aspect, the present invention provides a method of increasing the time interval between a first cardiovascular event and a second cardiovascular event in mammals, particularly humans, that have survived a first cardiovascular event comprising administering to the mammal or human, in need of such treatment, a therapeutically effective amount of a combination of alogliptin, sitagliptin, vildagliptin, or BI 1356, or a pharmaceutically acceptable salt thereof, a thiazolidinedione, and optionally at least one pharmaceutically acceptable carrier. Therapeutically effective amounts of alogliptin, sitagliptin, vildagliptin, or BI 1356 for combination with a thiazolidinedione for prolonging the survival time following a first cardiovascular event or for increasing the time interval between a first cardiovascular event and a second cardiovascular range from about 0.5 mgs/day to about 400 mgs/day. The preferred alogliptin, sitagliptin, vildagliptin, or BI 1356 doses for combination with a thiazolidinedione are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention provides a method of prolonging the survival time following a second cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, and a thiazolidinedione. In a further aspect, the present invention provides a method of increasing the time interval between a second cardiovascular event and a third cardiovascular event in mammals, particularly humans, that have survived a second cardiovascular event comprising administering to the mammal or human, in need of such treatment, a therapeutically effective amount of a combination of alogliptin, sitagliptin, vildagliptin, or BI 1356, or a pharmaceutically acceptable salt thereof, and a thiazolidinedione. Therapeutically effective amounts of alogliptin, sitagliptin, vildagliptin, or BI 1356 for combination with a thiazolidinedione for prolonging the survival time following a second cardiovascular event or for increasing the time interval between a second cardiovascular event and a third cardiovascular range from about 0.5 mgs/day to about 400 mgs/day. The preferred alogliptin, sitagliptin, vildagliptin, or BI 1356 doses for combination with a thiazolidinedione are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention relates to methods of preventing or reducing the risk of all cause mortality in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier. Therapeutically effective amounts of alogliptin, sitagliptin, vildagliptin, or BI 1356 for combination with rosiglitazone or pioglitazone range from about 0.5 mgs/day to about 400 mgs/day. The preferred alogliptin, sitagliptin, vildagliptin, or BI 1356 doses for combination with rosiglitazone or pioglitazone are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of alogliptin, sitagliptin, vildagliptin, or BI 1356, and rosiglitazone or pioglitazone prevent or reduce risk of all cause mortality in T2DM patients.

In another aspect, the present invention relates to methods of preventing or reducing the risk of all cause mortality in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier. Therapeutically effective amounts of alogliptin, sitagliptin, vildagliptin, or BI 1356 for combination with rosiglitazone or pioglitazone range from about 0.5 mgs/day to about 400 mgs/day. The preferred alogliptin, sitagliptin, vildagliptin, or BI 1356 doses for combination with rosiglitazone or pioglitazone are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of alogliptin, sitagliptin, vildagliptin, or BI 1356, and rosiglitazone or pioglitazone prevent or reduce risk of all cause mortality in T2DM patients that have a history of CV disease, a history of hypertension, a history of hypercholesterolemia, and/or a smoking history (current/previous).

In another aspect, the present invention relates to methods of preventing or reducing the risk of mortality caused by a cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier. Therapeutically effective amounts of alogliptin, sitagliptin, vildagliptin, or BI 1356 for combination with rosiglitazone or pioglitazone range from about 0.5 mgs/day to about 400 mgs/day. The preferred alogliptin, sitagliptin, vildagliptin, or BI 1356 doses for combination with rosiglitazone or pioglitazone are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of alogliptin, sitagliptin, vildagliptin, or BI 1356, and rosiglitazone or pioglitazone prevent or reduce risk of mortality caused by a CV event in T2DM patients.

In another aspect, the present invention relates to methods of preventing or reducing the risk of mortality caused by a cardiovascular event in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier. Therapeutically effective amounts of alogliptin, sitagliptin, vildagliptin, or BI 1356 for combination with rosiglitazone or pioglitazone range from about 0.5 mgs/day to about 400 mgs/day. The preferred alogliptin, sitagliptin, vildagliptin, or BI 1356 doses for combination with rosiglitazone or pioglitazone are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of alogliptin, sitagliptin, vildagliptin, or BI 1356, and rosiglitazone or pioglitazone prevent or reduce risk of mortality caused by a CV event in T2DM patients that have a history of CV disease, a history of hypertension, a history of hypercholesterolemia, and/or a smoking history (current/previous).

In another aspect, the present invention relates to methods of preventing or reducing the risk of non-fatal myocardial infarction and/or non-fatal stroke in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier. Therapeutically effective amounts of alogliptin, sitagliptin, vildagliptin, or BI 1356 for combination with rosiglitazone or pioglitazone range from about 0.5 mgs/day to about 400 mgs/day. The preferred alogliptin, sitagliptin, vildagliptin, or BI 1356 doses for combination with rosiglitazone or pioglitazone are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of alogliptin, sitagliptin, vildagliptin, or BI 1356, and rosiglitazone or pioglitazone prevent or reduce risk of non-fatal myocardial infarction and/or non-fatal stroke in T2DM patients.

In another aspect, the present invention relates to methods of preventing or reducing the risk of non-fatal myocardial infarction and/or non-fatal stroke in mammals, particularly humans, that have a history of CV disease, a history of hypertension, a history of hypercholesterolemia, and/or a smoking history (current/previous), comprising administering to the mammal or human, in need of such treatment, a therapeutically effective amount of a combination of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier. Therapeutically effective amounts of alogliptin, sitagliptin, vildagliptin, or BI 1356 for combination with rosiglitazone or pioglitazone range from about 0.5 mgs/day to about 400 mgs/day. The preferred alogliptin, sitagliptin, vildagliptin, or BI 1356 doses for combination with rosiglitazone or pioglitazone are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of alogliptin, sitagliptin, vildagliptin, or BI 1356 and rosiglitazone or pioglitazone prevent or reduce risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to methods of preventing or reducing the risk of mortality caused by a second cardiovascular event in mammals, particularly humans, that have survived a first cardiovascular event comprising administering to the mammal or human, in need of such treatment, a therapeutically effective amount of a combination of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier. Therapeutically effective amounts of alogliptin, sitagliptin, vildagliptin, or BI 1356 for combination with rosiglitazone or pioglitazone for preventing or reducing the risk of mortality caused by a second CV event range from about 0.5 mgs/day to about 400 mgs/day. The preferred alogliptin, sitagliptin, vildagliptin, or BI 1356 doses for combination with rosiglitazone or pioglitazone are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of alogliptin, sitagliptin, vildagliptin, or BI 1356, and rosiglitazone or pioglitazone prevent or reduce the risk of mortality caused by a second CV event in T2DM patients.

In another aspect, the present invention relates to methods of preventing or reducing the risk of mortality caused by a third cardiovascular event in mammals, particularly humans, that have survived two previous cardiovascular events comprising administering to the mammal or human, in need of such treatment, a therapeutically effective amount of a combination of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier. Therapeutically effective amounts of alogliptin, sitagliptin, vildagliptin, or BI 1356 for combination with rosiglitazone or pioglitazone for preventing or reducing the risk of mortality caused by a second CV event range from about 0.5 mgs/day to about 400 mgs/day. The preferred alogliptin, sitagliptin, vildagliptin, or BI 1356 doses for combination with rosiglitazone or pioglitazone are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Therapeutically effective amounts of the combination of alogliptin, sitagliptin, vildagliptin, or BI 1356 and rosiglitazone or pioglitazone prevent or reduce the risk of mortality caused by a third CV event in T2DM patients.

In another aspect, the present invention provides a method of prolonging the survival time following a first cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier. In a further aspect, the present invention provides a method of increasing the time interval between a first cardiovascular event and a second cardiovascular event in mammals, particularly humans, that have survived a first cardiovascular event comprising administering to the mammal or human, in need of such treatment, a therapeutically effective amount of a combination of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier. Therapeutically effective amounts of alogliptin, sitagliptin, vildagliptin, or BI 1356 for combination with rosiglitazone or pioglitazone for prolonging the survival time following a first cardiovascular event or for increasing the time interval between a first cardiovascular event and a second cardiovascular range from about 0.5 mgs/day to about 400 mgs/day. The preferred alogliptin, sitagliptin, vildagliptin, or BI 1356 doses for combination with rosiglitazone or pioglitazone are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl.

In another aspect, the present invention provides a method of prolonging the survival time following a second cardiovascular event in mammals, particularly humans, comprising administering to the mammal or human, in need of such treatment, a therapeutically effective amount of a combination of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier. In a further aspect, the present invention provides a method of increasing the time interval between a second cardiovascular event and a third cardiovascular event in mammals, particularly humans, that have survived a second cardiovascular event comprising administering to the mammal or human, in need of such treatment, a therapeutically effective amount of a combination of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier. Therapeutically effective amounts of alogliptin, sitagliptin, vildagliptin, or BI 1356 for combination with rosiglitazone or pioglitazone for prolonging the survival time following a second cardiovascular event or for increasing the time interval between a second cardiovascular event and a third cardiovascular range from about 0.5 mgs/day to about 400 mgs/day. The preferred alogliptin, sitagliptin, vildagliptin, or BI 1356 doses for combination with rosiglitazone or pioglitazone are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl.

In another aspect, the present invention relates to the use of saxagliptin, or a pharmaceutically acceptable salt, hydrate, or hydrate of a salt, thereof, and optionally at least one pharmaceutically acceptable carrier for the preparation or manufacture of a medicament for preventing or reducing the risk of all cause mortality in mammals, particularly humans. Amounts of saxagliptin for preparing or manufacturing a medicament useful for preventing or reducing the risk of all cause mortality range from about 0.5 mgs/day to about 400 mgs/day. Preferred saxagliptin amounts are 2.5 mgs/day, 5 mgs/day, and 10 mgs/day. Medicaments comprised of saxagliptin and optionally at least one pharmaceutically acceptable carrier are useful for preventing or reducing the risk of all cause mortality in T2DM patients.

In another aspect, the present invention relates to the use of saxagliptin, or a pharmaceutically acceptable salt, hydrate, or hydrate of a salt, thereof, and optionally at least one pharmaceutically acceptable carrier for the preparation or manufacture of a medicament for preventing or reducing the risk of all cause mortality in mammals, particularly humans, that have a history of CV disease, a history of hypertension, a history of hypercholesterolemia, and/or a smoking history (current/previous). Amounts of saxagliptin for preparing or manufacturing a medicament useful for preventing or reducing the risk of all cause mortality range from about 0.5 mgs/day to about 400 mgs/day. Preferred saxagliptin amounts are 2.5 mgs/day, 5 mgs/day, and 10 mgs/day. Medicaments comprised of saxagliptin and optionally at least one pharmaceutically acceptable carrier are useful for preventing or reducing the risk of all cause mortality in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, and optionally at least one pharmaceutically acceptable carrier for the preparation or manufacture of a medicament for preventing or reducing the risk of mortality caused by a cardiovascular event in mammals, particularly humans. Amounts of saxagliptin for preparing or manufacturing a medicament useful for preventing or reducing the risk of mortality caused by a cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin and optionally at least one pharmaceutically acceptable carrier are useful for preventing or reducing the risk of mortality caused by a cardiovascular event in T2DM patients.

In another aspect, the present invention relates to the use of saxagliptin, or a pharmaceutically acceptable salt, hydrate, or hydrate of a salt, thereof, and optionally at least one pharmaceutically acceptable carrier for the preparation or manufacture of a medicament for preventing or reducing the risk of mortality caused by a cardiovascular event in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of mortality caused by a cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin and optionally at least one pharmaceutically acceptable carrier are useful for preventing or reducing the risk of mortality caused by a cardiovascular event in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, and optionally at least one pharmaceutically acceptable carrier for the preparation or manufacture of a medicament for preventing or reducing the risk of non-fatal myocardial infarction and/or non-fatal stroke in mammals, particularly humans. Amounts of saxagliptin for preparing or manufacturing a medicament useful for preventing or reducing the risk of non-fatal myocardial infarction and/or non-fatal stroke range from about 0.5 mgs/day to about 400 mgs/day. Preferred saxagliptin amounts are 2.5 mgs/day, 5 mgs/day, and 10 mgs/day. Medicaments comprised of saxagliptin and optionally at least one pharmaceutically acceptable carrier are useful for preventing or reducing the risk of non-fatal myocardial infarction and/or non-fatal stroke in T2DM patients.

In another aspect, the present invention relates to the use of saxagliptin, or a pharmaceutically acceptable salt, hydrate, or hydrate of a salt, thereof, and optionally at least one pharmaceutically acceptable carrier for the preparation or manufacture of a medicament for preventing or reducing the risk of non-fatal myocardial infarction and/or non-fatal stroke in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of non-fatal myocardial infarction and/or non-fatal stroke range from about 0.5 mgs/day to about 400 mgs/day. Preferred saxagliptin amounts are 2.5 mgs/day, 5 mgs/day, and 10 mgs/day. Medicaments comprised of saxagliptin and optionally at least one pharmaceutically acceptable carrier are useful for preventing or reducing the risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, and optionally at least one pharmaceutically acceptable carrier for the preparation or manufacture of a medicament for preventing or reducing the risk of mortality caused by a second cardiovascular event in mammals, particularly humans, that have survived a first cardiovascular event. Amounts of saxagliptin for preparing or manufacturing a medicament useful for preventing or reducing the risk of mortality caused by a second cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin and optionally at least one pharmaceutically acceptable carrier are useful for preventing or reducing the risk of a second cardiovascular event in T2DM patients that have survived a first CV event.

In another aspect, the present invention relates to the use of saxagliptin, or a pharmaceutically acceptable salt, hydrate, or hydrate of a salt, thereof, and optionally at least one pharmaceutically acceptable carrier for the preparation or manufacture of a medicament for preventing or reducing the risk of mortality caused by a third cardiovascular event in mammals, particularly humans, that have survived two cardiovascular events. Amounts of saxagliptin for preparing or manufacturing a medicament useful for preventing or reducing the risk of mortality caused by a third cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin and optionally at least one pharmaceutically acceptable carrier are useful for preventing or reducing the risk of a third cardiovascular event in T2DM patients that have survived two CV events.

In another aspect, the present invention relates to the use of saxagliptin, or a pharmaceutically acceptable salt, hydrate, or hydrate of a salt, thereof, and optionally at least one pharmaceutically acceptable carrier for the preparation or manufacture of a medicament for prolonging the survival time following a first cardiovascular event in mammals, particularly humans. In a further aspect, the present invention provides the use of saxagliptin, or a pharmaceutically acceptable salt, hydrate, or hydrate of a salt, thereof, and optionally at least one pharmaceutically acceptable carrier for the preparation or manufacture of a medicament for increasing the time interval between a first cardiovascular event and a second cardiovascular event in mammals, particularly humans, that have survived a first cardiovascular event. Amounts of saxagliptin range from about 0.5 mgs/day to about 400 mgs/day. The preferred saxagliptin amounts are 2.5 mgs/day, 5 mgs/day, and 10 mgs/day.

In another aspect, the present invention relates to the use of saxagliptin, or a pharmaceutically acceptable salt, hydrate, or hydrate of a salt, thereof, and optionally at least one pharmaceutically acceptable carrier for the preparation or manufacture of a medicament for prolonging the survival time following a second cardiovascular event in mammals, particularly humans. In a further aspect, the present invention provides the use of saxagliptin, or a pharmaceutically acceptable salt, hydrate, or hydrate of a salt, thereof, and optionally at least one pharmaceutically acceptable carrier for the preparation or manufacture of a medicament for increasing the time interval between a second cardiovascular event and a third cardiovascular event in mammals, particularly humans, that have survived a second cardiovascular event. Amounts of saxagliptin range from about 0.5 mgs/day to about 400 mgs/day. The preferred saxagliptin amounts are 2.5 mgs/day, 5 mgs/day, and 10 mgs/day.

In another aspect, the present invention relates to the use of saxagliptin, or a pharmaceutically acceptable salt, hydrate, or hydrate of a salt, thereof, a sulfonyl urea, and optionally at least one pharmaceutically acceptable carrier for the preparation or manufacture of a medicament for preventing or reducing the risk of all cause mortality in mammals, particularly humans. Amounts of saxagliptin for preparing or manufacturing a medicament useful for preventing or reducing the risk of all cause mortality range from about 0.5 mgs/day to about 400 mgs/day. Preferred saxagliptin amounts are 2.5 mgs/day, 5 mgs/day, and 10 mgs/day. Medicaments comprised of saxagliptin, a sulfonylurea, and optionally at least one pharmaceutically acceptable carrier, are useful for preventing or reducing the risk of all cause mortality in T2DM patients.

In another aspect, the present invention relates to the use of saxagliptin, or a pharmaceutically acceptable salt, hydrate, or hydrate of a salt, thereof, a sulfonyl urea, and optionally at least one pharmaceutically acceptable carrier for the preparation or manufacture of a medicament for preventing or reducing the risk of all cause mortality in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of all cause mortality range from about 0.5 mgs/day to about 400 mgs/day. Preferred saxagliptin amounts are 2.5 mgs/day, 5 mgs/day, and 10 mgs/day. Medicaments comprised of saxagliptin, a sulfonylurea, and optionally at least one pharmaceutically acceptable carrier, are useful for preventing or reducing the risk of all cause mortality in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, a sulfonyl urea, and optionally at least one pharmaceutically acceptable carrier for the preparation or manufacture of a medicament for preventing or reducing the risk of mortality caused by a cardiovascular event in mammals, particularly humans. Amounts of saxagliptin for preparing or manufacturing a medicament useful for preventing or reducing the risk of mortality caused by a cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin, a sulfonylurea, and optionally at least one pharmaceutically acceptable carrier, are useful for preventing or reducing the risk of mortality caused by a cardiovascular event in T2DM patients.

In another aspect, the present invention relates to the use of saxagliptin, or a pharmaceutically acceptable salt, hydrate, or hydrate of a salt, thereof, a sulfonyl urea, and optionally at least one pharmaceutically acceptable carrier for the preparation or manufacture of a medicament for preventing or reducing the risk of mortality caused by a cardiovascular event in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of mortality caused by a cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin, a sulfonylurea, and optionally at least one pharmaceutically acceptable carrier, are useful for preventing or reducing the risk of mortality caused by a cardiovascular event in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, a sulfonylurea, and optionally at least one pharmaceutically acceptable carrier for the preparation or manufacture of a medicament for preventing or reducing the risk of non-fatal myocardial infarction and/or non-fatal stroke in mammals, particularly humans. Amounts of saxagliptin for preparing or manufacturing a medicament useful for preventing or reducing the risk of non-fatal myocardial infarction range from about 0.5 mgs/day to about 400 mgs/day. Preferred saxagliptin amounts are 2.5 mgs/day, 5 mgs/day, and 10 mgs/day. Medicaments comprised of saxagliptin, a sulfonylurea, and optionally at least one pharmaceutically acceptable carrier, are useful for preventing or reducing the risk of non-fatal myocardial infarction and/or non-fatal stroke in T2DM patients.

In another aspect, the present invention relates to the use of saxagliptin, or a pharmaceutically acceptable salt, hydrate, or hydrate of a salt, thereof, a sulfonylurea, and optionally at least one pharmaceutically acceptable carrier for the preparation or manufacture of a medicament for preventing or reducing the risk of non-fatal myocardial infarction and/or non-fatal stroke in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of non-fatal myocardial infarction range from about 0.5 mgs/day to about 400 mgs/day. Preferred saxagliptin amounts are 2.5 mgs/day, 5 mgs/day, and 10 mgs/day. Medicaments comprised of saxagliptin, a sulfonylurea, and optionally at least one pharmaceutically acceptable carrier, are useful for preventing or reducing the risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, a sulfonylurea, and optionally at least one pharmaceutically acceptable carrier, for the preparation or manufacture of a medicament for preventing or reducing the risk of mortality caused by a second cardiovascular event in mammals, particularly humans, that have survived a first cardiovascular event. Amounts of saxagliptin for preparing or manufacturing a medicament useful for preventing or reducing the risk of mortality caused by a second cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin, a sulfonylurea, and optionally at least one pharmaceutically acceptable carrier are useful for preventing or reducing the risk of a second cardiovascular event in T2DM patients that have survived a first CV event.

In another aspect, the present invention relates to the use of saxagliptin, or a pharmaceutically acceptable salt, hydrate, or hydrate of a salt, thereof, a sulfonylurea, and optionally at least one pharmaceutically acceptable carrier, for the preparation or manufacture of a medicament for preventing or reducing the risk of mortality caused by a third cardiovascular event in mammals, particularly humans, that have survived two cardiovascular events. Amounts of saxagliptin for preparing or manufacturing a medicament useful for preventing or reducing the risk of mortality caused by a third cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin, a sulfonylurea, and optionally at least one pharmaceutically acceptable carrier are useful for preventing or reducing the risk of a third cardiovascular event in mammals, particularly humans, that have survived two cardiovascular events, in T2DM patients.

In another aspect, the present invention relates to the use of a combination of saxagliptin, or a pharmaceutically acceptable salt, hydrate, or hydrate of a salt, thereof, a sulfonylurea, and optionally at least one pharmaceutically acceptable carrier for the preparation or manufacture of a medicament for prolonging the survival time following a first cardiovascular event in mammals, particularly humans. In a further aspect, the present invention provides the use of a combination of saxagliptin, or a pharmaceutically acceptable salt, hydrate, or hydrate of a salt, thereof, a sulfonylurea, and optionally at least one pharmaceutically acceptable carrier for the preparation or manufacture of a medicament for increasing the time interval between a first cardiovascular event and a second cardiovascular event in mammals, particularly humans, that have survived a first cardiovascular event. Amounts of saxagliptin range from about 0.5 mgs/day to about 400 mgs/day. The preferred saxagliptin amounts are 2.5 mgs/day, 5 mgs/day, and 10 mgs/day.

In another aspect, the present invention relates to the use of a combination of saxagliptin, or a pharmaceutically acceptable salt, hydrate, or hydrate of a salt, thereof, a sulfonylurea, and optionally at least one pharmaceutically acceptable carrier for the preparation or manufacture of a medicament for prolonging the survival time following a second cardiovascular event in mammals, particularly humans. In a further aspect, the present invention provides the use of a combination of saxagliptin, or a pharmaceutically acceptable salt, hydrate, or hydrate of a salt, thereof, a sulfonylurea, and optionally at least one pharmaceutically acceptable carrier for the preparation or manufacture of a medicament for increasing the time interval between a second cardiovascular event and a third cardiovascular event in mammals, particularly humans, that have survived a second cardiovascular event. Amounts of saxagliptin range from about 0.5 mgs/day to about 400 mgs/day. The preferred saxagliptin amounts are 2.5 mgs/day, 5 mgs/day, and 10 mgs/day.

In another aspect, the present invention relates to the use of saxagliptin, or a pharmaceutically acceptable salt, hydrate, or hydrate of a salt, thereof, glyburide, or a pharmaceutically acceptable salt thereof, and optionally at least one pharmaceutically acceptable carrier for the preparation or manufacture of a medicament for preventing or reducing the risk of all cause mortality in mammals, particularly humans. Amounts of saxagliptin for preparing or manufacturing a medicament useful for preventing or reducing the risk of all cause mortality range from about 0.5 mgs/day to about 400 mgs/day. Preferred saxagliptin amounts are 2.5 mgs/day, 5 mgs/day, and 10 mgs/day. The preferred dosing range for glyburide is about 0.5 mgs/day to about 15 mgs/day. Medicaments comprised of saxagliptin, glyburide, and optionally at least one pharmaceutically acceptable carrier, are useful for preventing or reducing the risk of all cause mortality in T2DM patients.

In another aspect, the present invention relates to the use of saxagliptin, or a pharmaceutically acceptable salt, hydrate, or hydrate of a salt, thereof, glyburide, or a pharmaceutically acceptable salt thereof, and optionally at least one pharmaceutically acceptable carrier for the preparation or manufacture of a medicament for preventing or reducing the risk of all cause mortality in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of all cause mortality range from about 0.5 mgs/day to about 400 mgs/day. Preferred saxagliptin amounts are 2.5 mgs/day, 5 mgs/day, and 10 mgs/day. The preferred dosing range for glyburide is about 0.5 mgs/day to about 15 mgs/day. Medicaments comprised of saxagliptin, glyburide, and optionally at least one pharmaceutically acceptable carrier, are useful for preventing or reducing the risk of all cause mortality in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, glyburide, or a pharmaceutically acceptable salt thereof, and optionally at least one pharmaceutically acceptable carrier for the preparation or manufacture of a medicament for preventing or reducing the risk of mortality caused by a cardiovascular event in mammals, particularly humans. Amounts of saxagliptin for preparing or manufacturing a medicament useful for preventing or reducing the risk of mortality caused by a cardiovascular event range from about 0.5 mgs/day to about 400 mgs/day. Preferred saxagliptin amounts are 2.5 mgs/day, 5 mgs/day, and 10 mgs/day. The preferred dosing range for glyburide is about 0.5 mgs/day to about 15 mgs/day. Medicaments comprised of saxagliptin, glyburide, and optionally at least one pharmaceutically acceptable carrier, are useful for preventing or reducing the risk of mortality caused by a cardiovascular event in T2DM patients.

In another aspect, the present invention relates to the use of saxagliptin, or a pharmaceutically acceptable salt, hydrate, or hydrate of a salt, thereof, glyburide, or a pharmaceutically acceptable salt thereof, and optionally at least one pharmaceutically acceptable carrier for the preparation or manufacture of a medicament for preventing or reducing the risk of mortality caused by a cardiovascular event in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of mortality caused by a cardiovascular event range from about 0.5 mgs/day to about 400 mgs/day. Preferred saxagliptin amounts are 2.5 mgs/day, 5 mgs/day, and 10 mgs/day. The preferred dosing range for glyburide is about 0.5 mgs/day to about 15 mgs/day. Medicaments comprised of saxagliptin, glyburide, and optionally at least one pharmaceutically acceptable carrier, are useful for preventing or reducing the risk of mortality caused by a cardiovascular event in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, glyburide, or a pharmaceutically acceptable salt thereof, and optionally at least one pharmaceutically acceptable carrier for the preparation or manufacture of a medicament for preventing or reducing the risk of non-fatal myocardial infarction and/or non-fatal stroke in mammals, particularly humans. Amounts of saxagliptin for preparing or manufacturing a medicament useful for preventing or reducing the risk of non-fatal myocardial infarction and/or non-fatal stroke range from about 0.5 mgs/day to about 400 mgs/day. Preferred saxagliptin amounts are 2.5 mgs/day, 5 mgs/day, and 10 mgs/day. The preferred dosing range for glyburide is about 0.5 mgs/day to about 15 mgs/day. Medicaments comprised of saxagliptin, glyburide, and optionally at least one pharmaceutically acceptable carrier, are useful for preventing or reducing the risk of non-fatal myocardial infarction and/or non-fatal stroke in T2DM patients.

In another aspect, the present invention relates to the use of saxagliptin, or a pharmaceutically acceptable salt, hydrate, or hydrate of a salt, thereof, glyburide, or a pharmaceutically acceptable salt thereof, and optionally at least one pharmaceutically acceptable carrier for the preparation or manufacture of a medicament for preventing or reducing the risk of non-fatal myocardial infarction and/or non-fatal stroke in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of non-fatal myocardial infarction and/or non-fatal stroke range from about 0.5 mgs/day to about 400 mgs/day. Preferred saxagliptin amounts are 2.5 mgs/day, 5 mgs/day, and 10 mgs/day. The preferred dosing range for glyburide is about 0.5 mgs/day to about 15 mgs/day. Medicaments comprised of saxagliptin, glyburide, and optionally at least one pharmaceutically acceptable carrier, are useful for preventing or reducing the risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, glyburide, or a pharmaceutically acceptable salt thereof, and optionally at least one pharmaceutically acceptable carrier, for the preparation or manufacture of a medicament for preventing or reducing the risk of mortality caused by a second cardiovascular event in mammals, particularly humans, that have survived a first cardiovascular event. Amounts of saxagliptin for preparing or manufacturing a medicament useful for preventing or reducing the risk of mortality caused by a second cardiovascular event range from about 0.5 mgs/day to about 400 mgs/day. Preferred saxagliptin amounts are 2.5 mgs/day, 5 mgs/day, and 10 mgs/day. The preferred dosing range for glyburide is about 0.5 mgs/day to about 15 mgs/day. Medicaments comprised of saxagliptin, glyburide, and optionally at least one pharmaceutically acceptable carrier are useful for preventing or reducing the risk of a second cardiovascular event in mammals, particularly humans, that have survived a first cardiovascular event, in T2DM patients that have survived a first CV event.

In another aspect, the present invention relates to the use of saxagliptin, or a pharmaceutically acceptable salt, hydrate, or hydrate of a salt, thereof, glyburide, or a pharmaceutically acceptable salt thereof, and optionally at least one pharmaceutically acceptable carrier, for the preparation or manufacture of a medicament for preventing or reducing the risk of mortality caused by a third cardiovascular event in mammals, particularly humans, that have survived two cardiovascular events. Amounts of saxagliptin for preparing or manufacturing a medicament useful for preventing or reducing the risk of mortality caused by a third cardiovascular event range from about 0.5 mgs/day to about 400 mgs/day. Preferred saxagliptin amounts are 2.5 mgs/day, 5 mgs/day, and 10 mgs/day. The preferred dosing range for glyburide is about 0.5 mgs/day to about 15 mgs/day. Medicaments comprised of saxagliptin, glyburide, and optionally at least one pharmaceutically acceptable carrier are useful for preventing or reducing the risk of a third cardiovascular event in T2DM patients that have survived two CV events.

In another aspect, the present invention relates to the use of a combination of saxagliptin, or a pharmaceutically acceptable salt, hydrate, or hydrate of a salt, thereof, glyburide, or a pharmaceutically acceptable salt thereof, and optionally at least one pharmaceutically acceptable carrier for the preparation or manufacture of a medicament for prolonging the survival time following a first cardiovascular event in mammals, particularly humans. In a further aspect, the present invention provides the use of a combination of saxagliptin, or a pharmaceutically acceptable salt, hydrate, or hydrate of a salt, thereof, glyburide, or a pharmaceutically acceptable salt thereof, and optionally at least one pharmaceutically acceptable carrier for the preparation or manufacture of a medicament for increasing the time interval between a first cardiovascular event and a second cardiovascular event in mammals, particularly humans, that have survived a first cardiovascular event. Amounts of saxagliptin range from about 0.5 mgs/day to about 400 mgs/day. The preferred saxagliptin amounts are 2.5 mgs/day, 5 mgs/day, and 10 mgs/day. The preferred dosing range for glyburide is about 0.5 mgs/day to about 15 mgs/day.

In another aspect, the present invention relates to the use of a combination of saxagliptin, or a pharmaceutically acceptable salt, hydrate, or hydrate of a salt, thereof, glyburide, or a pharmaceutically acceptable salt thereof, and optionally at least one pharmaceutically acceptable carrier for the preparation or manufacture of a medicament for prolonging the survival time following a second cardiovascular event in mammals, particularly humans. In a further aspect, the present invention provides the use of a combination of saxagliptin, or a pharmaceutically acceptable salt, hydrate, or hydrate of a salt, thereof, glyburide, or a pharmaceutically acceptable salt thereof, and optionally at least one pharmaceutically acceptable carrier for the preparation or manufacture of a medicament for increasing the time interval between a second cardiovascular event and a third cardiovascular event in mammals, particularly humans, that have survived a second cardiovascular event. Amounts of saxagliptin range from about 0.5 mgs/day to about 400 mgs/day. The preferred saxagliptin amounts are 2.5 mgs/day, 5 mgs/day, and 10 mgs/day. The preferred dosing range for glyburide is about 0.5 mgs/day to about 15 mgs/day.

In another aspect, the present invention relates to the use of saxagliptin, or a pharmaceutically acceptable salt, hydrate, or hydrate of a salt, thereof, a biguanide, and optionally at least one pharmaceutically acceptable carrier for the preparation or manufacture of a medicament for preventing or reducing the risk of all cause mortality in mammals, particularly humans. Amounts of saxagliptin for preparing or manufacturing a medicament useful for preventing or reducing the risk of all cause mortality range from about 0.5 mgs/day to about 400 mgs/day. Preferred saxagliptin amounts are 2.5 mgs/day, 5 mgs/day, and 10 mgs/day. Medicaments comprised of saxagliptin, a biguanide, and optionally at least one pharmaceutically acceptable carrier, are useful for preventing or reducing the risk of all cause mortality in T2DM patients.

In another aspect, the present invention relates to the use of saxagliptin, or a pharmaceutically acceptable salt, hydrate, or hydrate of a salt, thereof, a biguanide, and optionally at least one pharmaceutically acceptable carrier for the preparation or manufacture of a medicament for preventing or reducing the risk of all cause mortality in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of all cause mortality range from about 0.5 mgs/day to about 400 mgs/day. Preferred saxagliptin amounts are 2.5 mgs/day, 5 mgs/day, and 10 mgs/day. Medicaments comprised of saxagliptin, a biguanide, and optionally at least one pharmaceutically acceptable carrier, are useful for preventing or reducing the risk of all cause mortality in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, a biguanide, and optionally at least one pharmaceutically acceptable carrier for the preparation or manufacture of a medicament for preventing or reducing the risk of mortality caused by a cardiovascular event in mammals, particularly humans. Amounts of saxagliptin for preparing or manufacturing a medicament useful for preventing or reducing the risk of mortality caused by a cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin, a biguanide, and optionally at least one pharmaceutically acceptable carrier, are useful for preventing or reducing the risk of mortality caused by a cardiovascular event in T2DM patients.

In another aspect, the present invention relates to the use of saxagliptin, or a pharmaceutically acceptable salt, hydrate, or hydrate of a salt, thereof, a biguanide, and optionally at least one pharmaceutically acceptable carrier for the preparation or manufacture of a medicament for preventing or reducing the risk of mortality caused by a cardiovascular event in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of mortality caused by a cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin, a biguanide, and optionally at least one pharmaceutically acceptable carrier, are useful for preventing or reducing the risk of mortality caused by a cardiovascular event in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, a biguanide, and optionally at least one pharmaceutically acceptable carrier for the preparation or manufacture of a medicament for preventing or reducing the risk of non-fatal myocardial infarction and/or non-fatal stroke in mammals, particularly humans. Amounts of saxagliptin for preparing or manufacturing a medicament useful for preventing or reducing the risk of non-fatal myocardial infarction and/or non-fatal stroke range from about 0.5 mgs/day to about 400 mgs/day. Preferred saxagliptin amounts are 2.5 mgs/day, 5 mgs/day, and 10 mgs/day. Medicaments comprised of saxagliptin, a biguanide, and optionally at least one pharmaceutically acceptable carrier, are useful for preventing or reducing the risk of non-fatal myocardial infarction and/or non-fatal stroke in T2DM patients.

In another aspect, the present invention relates to the use of saxagliptin, or a pharmaceutically acceptable salt, hydrate, or hydrate of a salt, thereof, a biguanide, and optionally at least one pharmaceutically acceptable carrier for the preparation or manufacture of a medicament for preventing or reducing the risk of non-fatal myocardial infarction and/or non-fatal stroke in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of non-fatal myocardial infarction and/or non-fatal stroke range from about 0.5 mgs/day to about 400 mgs/day. Preferred saxagliptin amounts are 2.5 mgs/day, 5 mgs/day, and 10 mgs/day. Medicaments comprised of saxagliptin, a biguanide, and optionally at least one pharmaceutically acceptable carrier, are useful for preventing or reducing the risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, a biguanide, and optionally at least one pharmaceutically acceptable carrier, for the preparation or manufacture of a medicament for preventing or reducing the risk of mortality caused by a second cardiovascular event in mammals, particularly humans, that have survived a first cardiovascular event. Amounts of saxagliptin for preparing or manufacturing a medicament useful for preventing or reducing the risk of mortality caused by a second cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin, a biguanide, and optionally at least one pharmaceutically acceptable carrier are useful for preventing or reducing the risk of a second cardiovascular event in T2DM patients that have survived a first CV event.

In another aspect, the present invention relates to the use of saxagliptin, or a pharmaceutically acceptable salt, hydrate, or hydrate of a salt, thereof, a biguanide, and optionally at least one pharmaceutically acceptable carrier, for the preparation or manufacture of a medicament for preventing or reducing the risk of mortality caused by a third cardiovascular event in mammals, particularly humans, that have survived two cardiovascular events. Amounts of saxagliptin for preparing or manufacturing a medicament useful for preventing or reducing the risk of mortality caused by a third cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin, a biguanide, and optionally at least one pharmaceutically acceptable carrier are useful for preventing or reducing the risk of a third cardiovascular event in T2DM patients that have survived two CV events.

In another aspect, the present invention relates to the use of a combination of saxagliptin, or a pharmaceutically acceptable salt, hydrate, or hydrate of a salt, thereof, a biguanide, and optionally at least one pharmaceutically acceptable carrier for the preparation or manufacture of a medicament for prolonging the survival time following a first cardiovascular event in mammals, particularly humans. In a further aspect, the present invention provides the use of a combination of saxagliptin, or a pharmaceutically acceptable salt, hydrate, or hydrate of a salt, thereof, a biguanide, and optionally at least one pharmaceutically acceptable carrier for the preparation or manufacture of a medicament for increasing the time interval between a first cardiovascular event and a second cardiovascular event in mammals, particularly humans, that have survived a first cardiovascular event. Amounts of saxagliptin range from about 0.5 mgs/day to about 400 mgs/day. The preferred saxagliptin amounts are 2.5 mgs/day, 5 mgs/day, and 10 mgs/day.

In another aspect, the present invention relates to the use of a combination of saxagliptin, or a pharmaceutically acceptable salt, hydrate, or hydrate of a salt, thereof, a biguanide, and optionally at least one pharmaceutically acceptable carrier for the preparation or manufacture of a medicament for prolonging the survival time following a second cardiovascular event in mammals, particularly humans. In a further aspect, the present invention provides the use of a combination of saxagliptin, or a pharmaceutically acceptable salt, hydrate, or hydrate of a salt, thereof, a biguanide, and optionally at least one pharmaceutically acceptable carrier for the preparation or manufacture of a medicament for increasing the time interval between a second cardiovascular event and a third cardiovascular event in mammals, particularly humans, that have survived a second cardiovascular event. Amounts of saxagliptin range from about 0.5 mgs/day to about 400 mgs/day. The preferred saxagliptin amounts are 2.5 mgs/day, 5 mgs/day, and 10 mgs/day.

In another aspect, the present invention relates to the use of saxagliptin, or a pharmaceutically acceptable salt, hydrate, or hydrate of a salt, thereof, metformin, or a pharmaceutically acceptable salt thereof, and optionally at least one pharmaceutically acceptable carrier for the preparation or manufacture of a medicament for preventing or reducing the risk of all cause mortality in mammals, particularly humans. Amounts of saxagliptin for preparing or manufacturing a medicament useful for preventing or reducing the risk of all cause mortality range from about 0.5 mgs/day to about 400 mgs/day. Preferred saxagliptin amounts are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Medicaments comprised of saxagliptin, metformin, and optionally at least one pharmaceutically acceptable carrier, are useful for preventing or reducing the risk of all cause mortality in T2DM patients.

In another aspect, the present invention relates to the use of saxagliptin, or a pharmaceutically acceptable salt, hydrate, or hydrate of a salt, thereof, metformin, or a pharmaceutically acceptable salt thereof, and optionally at least one pharmaceutically acceptable carrier for the preparation or manufacture of a medicament for preventing or reducing the risk of all cause mortality in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of all cause mortality range from about 0.5 mgs/day to about 400 mgs/day. Preferred saxagliptin amounts are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Medicaments comprised of saxagliptin, metformin, and optionally at least one pharmaceutically acceptable carrier, are useful for preventing or reducing the risk of all cause mortality in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, metformin, or a pharmaceutically acceptable salt thereof, and optionally at least one pharmaceutically acceptable carrier for the preparation or manufacture of a medicament for preventing or reducing the risk of mortality caused by a cardiovascular event in mammals, particularly humans. Amounts of saxagliptin for preparing or manufacturing a medicament useful for preventing or reducing the risk of mortality caused by a cardiovascular event range from about 0.5 mgs/day to about 400 mgs/day. Preferred saxagliptin amounts are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Medicaments comprised of saxagliptin, metformin, and optionally at least one pharmaceutically acceptable carrier, are useful for preventing or reducing the risk of mortality caused by a cardiovascular event in T2DM patients.

In another aspect, the present invention relates to the use of saxagliptin, or a pharmaceutically acceptable salt, hydrate, or hydrate of a salt, thereof, metformin, or a pharmaceutically acceptable salt thereof, and optionally at least one pharmaceutically acceptable carrier for the preparation or manufacture of a medicament for preventing or reducing the risk of mortality caused by a cardiovascular event in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of mortality caused by a cardiovascular event range from about 0.5 mgs/day to about 400 mgs/day. Preferred saxagliptin amounts are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Medicaments comprised of saxagliptin, metformin, and optionally at least one pharmaceutically acceptable carrier, are useful for preventing or reducing the risk of mortality caused by a cardiovascular event in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, metformin, or a pharmaceutically acceptable salt thereof, and optionally at least one pharmaceutically acceptable carrier for the preparation or manufacture of a medicament for preventing or reducing the risk of non-fatal myocardial infarction and/or non-fatal stroke in mammals, particularly humans. Amounts of saxagliptin for preparing or manufacturing a medicament useful for preventing or reducing the risk of non-fatal myocardial infarction and/or non-fatal stroke range from about 0.5 mgs/day to about 400 mgs/day. Preferred saxagliptin amounts are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Medicaments comprised of saxagliptin, metformin, and optionally at least one pharmaceutically acceptable carrier, are useful for preventing or reducing the risk of non-fatal myocardial infarction and/or non-fatal stroke in T2DM patients.

In another aspect, the present invention relates to the use of saxagliptin, or a pharmaceutically acceptable salt, hydrate, or hydrate of a salt, thereof, metformin, or a pharmaceutically acceptable salt thereof, and optionally at least one pharmaceutically acceptable carrier for the preparation or manufacture of a medicament for preventing or reducing the risk of non-fatal myocardial infarction and/or non-fatal stroke in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of non-fatal myocardial infarction and/or non-fatal stroke range from about 0.5 mgs/day to about 400 mgs/day. Preferred saxagliptin amounts are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Medicaments comprised of saxagliptin, metformin, and optionally at least one pharmaceutically acceptable carrier, are useful for preventing or reducing the risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, metformin, or a pharmaceutically acceptable salt thereof, and optionally at least one pharmaceutically acceptable carrier, for the preparation or manufacture of a medicament for preventing or reducing the risk of mortality caused by a second cardiovascular event in mammals, particularly humans, that have survived a first cardiovascular event. Amounts of saxagliptin for preparing or manufacturing a medicament useful for preventing or reducing the risk of mortality caused by a second cardiovascular event range from about 0.5 mgs/day to about 400 mgs/day. Preferred saxagliptin amounts are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Medicaments comprised of saxagliptin, metformin, and optionally at least one pharmaceutically acceptable carrier are useful for preventing or reducing the risk of a second cardiovascular event in T2DM patients that have survived a first CV event.

In another aspect, the present invention relates to the use of saxagliptin, or a pharmaceutically acceptable salt, hydrate, or hydrate of a salt, thereof, metformin, or a pharmaceutically acceptable salt thereof, and optionally at least one pharmaceutically acceptable carrier, for the preparation or manufacture of a medicament for preventing or reducing the risk of mortality caused by a third cardiovascular event in mammals, particularly humans, that have survived two cardiovascular events. Amounts of saxagliptin for preparing or manufacturing a medicament useful for preventing or reducing the risk of mortality caused by a third cardiovascular event range from about 0.5 mgs/day to about 400 mgs/day. Preferred saxagliptin amounts are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl. Medicaments comprised of saxagliptin, metformin, and optionally at least one pharmaceutically acceptable carrier are useful for preventing or reducing the risk of a third cardiovascular event in T2DM patients that have survived two CV events.

In another aspect, the present invention relates to the use of a combination of saxagliptin, or a pharmaceutically acceptable salt, hydrate, or hydrate of a salt, thereof, metformin, or a pharmaceutically acceptable salt thereof, and optionally at least one pharmaceutically acceptable carrier for the preparation or manufacture of a medicament for prolonging the survival time following a first cardiovascular event in mammals, particularly humans. In a further aspect, the present invention provides the use of a combination of saxagliptin, or a pharmaceutically acceptable salt, hydrate, or hydrate of a salt, thereof, metformin, or a pharmaceutically acceptable salt thereof, and optionally at least one pharmaceutically acceptable carrier for the preparation or manufacture of a medicament for increasing the time interval between a first cardiovascular event and a second cardiovascular event in mammals, particularly humans, that have survived a first cardiovascular event. Amounts of saxagliptin range from about 0.5 mgs/day to about 400 mgs/day. The preferred saxagliptin amounts are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl.

In another aspect, the present invention relates to the use of a combination of saxagliptin, or a pharmaceutically acceptable salt, hydrate, or hydrate of a salt, thereof, metformin, or a pharmaceutically acceptable salt thereof, and optionally at least one pharmaceutically acceptable carrier for the preparation or manufacture of a medicament for prolonging the survival time following a second cardiovascular event in mammals, particularly humans. In a further aspect, the present invention provides the use of a combination of saxagliptin, or a pharmaceutically acceptable salt, hydrate, or hydrate of a salt, thereof, metformin, or a pharmaceutically acceptable salt thereof, and optionally at least one pharmaceutically acceptable carrier for the preparation or manufacture of a medicament for increasing the time interval between a second cardiovascular event and a third cardiovascular event in mammals, particularly humans, that have survived a second cardiovascular event. Amounts of saxagliptin range from about 0.5 mgs/day to about 400 mgs/day. The preferred saxagliptin amounts are 2.5 mgs/day, 5 mgs/day, and 10 mgs/day. The preferred dosing range for metformin is about 100 mgs/day to about 2500 mgs/day. The preferred pharmaceutically acceptable salt for metformin is HCl.

In another aspect, the present invention relates to the use of saxagliptin, or a pharmaceutically acceptable salt, hydrate, or hydrate of a salt, thereof, a thiazolidinedione, and optionally at least one pharmaceutically acceptable carrier for the preparation or manufacture of a medicament for preventing or reducing the risk of all cause mortality in mammals, particularly humans. Amounts of saxagliptin for preparing or manufacturing a medicament useful for preventing or reducing the risk of all cause mortality range from about 0.5 mgs/day to about 400 mgs/day. Preferred saxagliptin amounts are 2.5 mgs/day, 5 mgs/day, and 10 mgs/day. Medicaments comprised of saxagliptin, a thiazolidinedione, and optionally at least one pharmaceutically acceptable carrier, are useful for preventing or reducing the risk of all cause mortality in T2DM patients.

In another aspect, the present invention relates to the use of saxagliptin, or a pharmaceutically acceptable salt, hydrate, or hydrate of a salt, thereof, a thiazolidinedione, and optionally at least one pharmaceutically acceptable carrier for the preparation or manufacture of a medicament for preventing or reducing the risk of all cause mortality in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of all cause mortality range from about 0.5 mgs/day to about 400 mgs/day. Preferred saxagliptin amounts are 2.5 mgs/day, 5 mgs/day, and 10 mgs/day. Medicaments comprised of saxagliptin, a thiazolidinedione, and optionally at least one pharmaceutically acceptable carrier, are useful for preventing or reducing the risk of all cause mortality in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, a thiazolidinedione, and optionally at least one pharmaceutically acceptable carrier for the preparation or manufacture of a medicament for preventing or reducing the risk of mortality caused by a cardiovascular event in mammals, particularly humans. Amounts of saxagliptin for preparing or manufacturing a medicament useful for preventing or reducing the risk of mortality caused by a cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin, a thiazolidinedione, and optionally at least one pharmaceutically acceptable carrier, are useful for preventing or reducing the risk of mortality caused by a cardiovascular event in T2DM patients.

In another aspect, the present invention relates to the use of saxagliptin, or a pharmaceutically acceptable salt, hydrate, or hydrate of a salt, thereof, a thiazolidinedione, and optionally at least one pharmaceutically acceptable carrier for the preparation or manufacture of a medicament for preventing or reducing the risk of mortality caused by a cardiovascular event in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of mortality caused by a cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin, a thiazolidinedione, and optionally at least one pharmaceutically acceptable carrier, are useful for preventing or reducing the risk of mortality caused by a cardiovascular event in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, a thiazolidinedione, and optionally at least one pharmaceutically acceptable carrier for the preparation or manufacture of a medicament for preventing or reducing the risk of non-fatal myocardial infarction and/or non-fatal stroke in mammals, particularly humans. Amounts of saxagliptin for preparing or manufacturing a medicament useful for preventing or reducing the risk of non-fatal myocardial infarction range from about 0.5 mgs/day to about 400 mgs/day. Preferred saxagliptin amounts are 2.5 mgs/day, 5 mgs/day, and 10 mgs/day. Medicaments comprised of saxagliptin, a thiazolidinedione, and optionally at least one pharmaceutically acceptable carrier, are useful for preventing or reducing the risk of non-fatal myocardial infarction and/or non-fatal stroke in T2DM patients.

In another aspect, the present invention relates to the use of saxagliptin, or a pharmaceutically acceptable salt, hydrate, or hydrate of a salt, thereof, a thiazolidinedione, and optionally at least one pharmaceutically acceptable carrier for the preparation or manufacture of a medicament for preventing or reducing the risk of non-fatal myocardial infarction and/or non-fatal stroke in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of non-fatal myocardial infarction range from about 0.5 mgs/day to about 400 mgs/day. Preferred saxagliptin amounts are 2.5 mgs/day, 5 mgs/day, and 10 mgs/day. Medicaments comprised of saxagliptin, a thiazolidinedione, and optionally at least one pharmaceutically acceptable carrier, are useful for preventing or reducing the risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, a thiazolidinedione, and optionally at least one pharmaceutically acceptable carrier, for the preparation or manufacture of a medicament for preventing or reducing the risk of mortality caused by a second cardiovascular event in mammals, particularly humans, that have survived a first cardiovascular event. Amounts of saxagliptin for preparing or manufacturing a medicament useful for preventing or reducing the risk of mortality caused by a second cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin, a thiazolidinedione, and optionally at least one pharmaceutically acceptable carrier are useful for preventing or reducing the risk of a second cardiovascular event in T2DM patients that have survived a first CV event.

In another aspect, the present invention relates to the use of saxagliptin, or a pharmaceutically acceptable salt, hydrate, or hydrate of a salt, thereof, a thiazolidinedione, and optionally at least one pharmaceutically acceptable carrier, for the preparation or manufacture of a medicament for preventing or reducing the risk of mortality caused by a third cardiovascular event in mammals, particularly humans, that have survived two cardiovascular events. Amounts of saxagliptin for preparing or manufacturing a medicament useful for preventing or reducing the risk of mortality caused by a third cardiovascular event range from about 0.5 mgs/day to about 400 mgs/day. Preferred saxagliptin amounts are 2.5 mgs/day, 5 mgs/day, and 10 mgs/day. Medicaments comprised of saxagliptin, a thiazolidinedione, and optionally at least one pharmaceutically acceptable carrier are useful for preventing or reducing the risk of a third cardiovascular event in mammals, particularly humans, that have survived two cardiovascular events, in T2DM patients.

In another aspect, the present invention relates to the use of a combination of saxagliptin, or a pharmaceutically acceptable salt, hydrate, or hydrate of a salt, thereof, a thiazolidinedione, and optionally at least one pharmaceutically acceptable carrier for the preparation or manufacture of a medicament for prolonging the survival time following a first cardiovascular event in mammals, particularly humans. In a further aspect, the present invention provides the use of a combination of saxagliptin, or a pharmaceutically acceptable salt, hydrate, or hydrate of a salt, thereof, a thiazolidinedione, and optionally at least one pharmaceutically acceptable carrier for the preparation or manufacture of a medicament for increasing the time interval between a first cardiovascular event and a second cardiovascular event in mammals, particularly humans, that have survived a first cardiovascular event. Amounts of saxagliptin range from about 0.5 mgs/day to about 400 mgs/day. The preferred saxagliptin amounts are 2.5 mgs/day, 5 mgs/day, and 10 mgs/day.

In another aspect, the present invention relates to the use of a combination of saxagliptin, or a pharmaceutically acceptable salt, hydrate, or hydrate of a salt, thereof, a thiazolidinedione, and optionally at least one pharmaceutically acceptable carrier for the preparation or manufacture of a medicament for prolonging the survival time following a second cardiovascular event in mammals, particularly humans. In a further aspect, the present invention provides the use of a combination of saxagliptin, or a pharmaceutically acceptable salt, hydrate, or hydrate of a salt, thereof, a thiazolidinedione, and optionally at least one pharmaceutically acceptable carrier for the preparation or manufacture of a medicament for increasing the time interval between a second cardiovascular event and a third cardiovascular event in mammals, particularly humans, that have survived a second cardiovascular event. Amounts of saxagliptin range from about 0.5 mgs/day to about 400 mgs/day. The preferred saxagliptin amounts are 2.5 mgs/day, 5 mgs/day, and 10 mgs/day.

In another aspect, the present invention relates to the use of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier for the preparation or manufacture of a medicament for preventing or reducing the risk of all cause mortality in mammals, particularly humans. Amounts of saxagliptin for preparing or manufacturing a medicament useful for preventing or reducing the risk of all cause mortality range from about 0.5 mgs/day to about 400 mgs/day. Preferred saxagliptin amounts are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of saxagliptin and rosiglitazone or saxagliptin and pioglitazone, and optionally at least one pharmaceutically acceptable carrier, are useful for preventing or reducing the risk of all cause mortality in T2DM patients.

In another aspect, the present invention relates to the use of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier for the preparation or manufacture of a medicament for preventing or reducing the risk of all cause mortality in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of all cause mortality range from about 0.5 mgs/day to about 400 mgs/day. Preferred saxagliptin amounts are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of saxagliptin and rosiglitazone or saxagliptin and pioglitazone, and optionally at least one pharmaceutically acceptable carrier, are useful for preventing or reducing the risk of all cause mortality in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier for the preparation or manufacture of a medicament for preventing or reducing the risk of mortality caused by a cardiovascular event in mammals, particularly humans. Amounts of saxagliptin for preparing or manufacturing a medicament useful for preventing or reducing the risk of mortality caused by a cardiovascular event range from about 0.5 mgs/day to about 400 mgs/day. Preferred saxagliptin amounts are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of saxagliptin and rosiglitazone or saxagliptin and pioglitazone, and optionally at least one pharmaceutically acceptable carrier, are useful for preventing or reducing the risk of mortality caused by a cardiovascular event in T2DM patients.

In another aspect, the present invention relates to the use of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier for the preparation or manufacture of a medicament for preventing or reducing the risk of mortality caused by a cardiovascular event in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of mortality caused by a cardiovascular event range from about 0.5 mgs/day to about 400 mgs/day. Preferred saxagliptin amounts are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of saxagliptin and rosiglitazone or saxagliptin and pioglitazone, and optionally at least one pharmaceutically acceptable carrier, are useful for preventing or reducing the risk of mortality caused by a cardiovascular event in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier for the preparation or manufacture of a medicament for preventing or reducing the risk of non-fatal myocardial infarction and/or non-fatal stroke in mammals, particularly humans. Amounts of saxagliptin for preparing or manufacturing a medicament useful for preventing or reducing the risk of non-fatal myocardial infarction and/or non-fatal stroke range from about 0.5 mgs/day to about 400 mgs/day. Preferred saxagliptin amounts are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of saxagliptin and rosiglitazone or saxagliptin and pioglitazone, and optionally at least one pharmaceutically acceptable carrier, are useful for preventing or reducing the risk of non-fatal myocardial infarction and/or non-fatal stroke in T2DM patients.

In another aspect, the present invention relates to the use of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier for the preparation or manufacture of a medicament for preventing or reducing the risk of non-fatal myocardial infarction and/or non-fatal stroke in mammals, particularly humans that have a history of CV disease, a history of hypertension, a history of hypercholesterolemia, and/or a smoking history (current/previous). Amounts of saxagliptin for preparing or manufacturing a medicament useful for preventing or reducing the risk of non-fatal myocardial infarction and/or non-fatal stroke range from about 0.5 mgs/day to about 400 mgs/day. Preferred saxagliptin amounts are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of saxagliptin and rosiglitazone or saxagliptin and pioglitazone, and optionally at least one pharmaceutically acceptable carrier, are useful for preventing or reducing the risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to the use of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier, for the preparation or manufacture of a medicament for preventing or reducing the risk of mortality caused by a second cardiovascular event in mammals, particularly humans, that have survived a first cardiovascular event. Amounts of saxagliptin for preparing or manufacturing a medicament useful for preventing or reducing the risk of mortality caused by a second cardiovascular event range from about 0.5 mgs/day to about 400 mgs/day. Preferred saxagliptin amounts are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of saxagliptin and rosiglitazone or saxagliptin and pioglitazone, and optionally at least one pharmaceutically acceptable carrier are useful for preventing or reducing the risk of a second cardiovascular event in mammals, particularly humans, that have survived a first cardiovascular event, in T2DM patients that have survived a first cardiovascular event.

In another aspect, the present invention relates to the use of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier, for the preparation or manufacture of a medicament for preventing or reducing the risk of mortality caused by a third cardiovascular event in mammals, particularly humans, that have survived two cardiovascular events. Amounts of saxagliptin for preparing or manufacturing a medicament useful for preventing or reducing the risk of mortality caused by a third cardiovascular event range from about 0.5 mgs/day to about 400 mgs/day. Preferred saxagliptin amounts are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of saxagliptin and rosiglitazone or saxagliptin and pioglitazone, and optionally at least one pharmaceutically acceptable carrier are useful for preventing or reducing the risk of a third cardiovascular event in T2DM patients that have survived two cardiovascular events.

In another aspect, the present invention relates to the use of a combination of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier for the preparation or manufacture of a medicament for prolonging the survival time following a first cardiovascular event in mammals, particularly humans. In a further aspect, the present invention provides the use of a combination of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier for the preparation or manufacture of a medicament for increasing the time interval between a first cardiovascular event and a second cardiovascular event in mammals, particularly humans, that have survived a first cardiovascular event. Amounts of saxagliptin range from about 0.5 mgs/day to about 400 mgs/day. The preferred saxagliptin amounts are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl.

In another aspect, the present invention relates to the use of a combination of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier for the preparation or manufacture of a medicament for prolonging the survival time following a second cardiovascular event in mammals, particularly humans. In a further aspect, the present invention provides the use of a combination of saxagliptin, or a pharmaceutically acceptable salt, hydrate, or hydrate of a salt, thereof, rosiglitazone or pioglitazone, or a pharmaceutically acceptable salt thereof, and optionally at least one pharmaceutically acceptable carrier for the preparation or manufacture of a medicament for increasing the time interval between a second cardiovascular event and a third cardiovascular event in mammals, particularly humans, that have survived a second cardiovascular event. Amounts of saxagliptin range from about 0.5 mgs/day to about 400 mgs/day. The preferred saxagliptin amounts are 2.5 mgs/day, 5 mgs/day, and 10 mgs/day. The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl.

In another aspect, the present invention relates to the use of alogliptin, sitagliptin, vildagliptin, or BI 1356, or a pharmaceutically acceptable salt thereof, and optionally at least one pharmaceutically acceptable carrier for the preparation or manufacture of a medicament for preventing or reducing the risk of all cause mortality in mammals, particularly humans. Amounts of alogliptin, sitagliptin, vildagliptin, or BI 1356 for preparing or manufacturing a medicament useful for preventing or reducing the risk of all cause mortality range from about 0.5 mgs/day to about 400 mgs/day. Preferred alogliptin, sitagliptin, vildagliptin, or BI 1356 amounts are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356 and optionally at least one pharmaceutically acceptable carrier are useful for preventing or reducing the risk of all cause mortality in T2DM patients.

In another aspect, the present invention relates to the use of alogliptin, sitagliptin, vildagliptin, or BI 1356, or a pharmaceutically acceptable salt thereof, and optionally at least one pharmaceutically acceptable carrier for the preparation or manufacture of a medicament for preventing or reducing the risk of all cause mortality in mammals, particularly humans that have a history of CV disease, a history of hypertension, a history of hypercholesterolemia, and/or a smoking history (current/previous). Amounts of alogliptin, sitagliptin, vildagliptin, or BI 1356 for preparing or manufacturing a medicament useful for preventing or reducing the risk of all cause mortality range from about 0.5 mgs/day to about 400 mgs/day. Preferred alogliptin, sitagliptin, vildagliptin, or BI 1356 amounts are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356 and optionally at least one pharmaceutically acceptable carrier are useful for preventing or reducing the risk of all cause mortality in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, and optionally at least one pharmaceutically acceptable carrier for the preparation or manufacture of a medicament for preventing or reducing the risk of mortality caused by a cardiovascular event in mammals, particularly humans. Amounts of alogliptin, sitagliptin, vildagliptin, or BI 1356 for preparing or manufacturing a medicament useful for preventing or reducing the risk of mortality caused by a cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356 and optionally at least one pharmaceutically acceptable carrier are useful for preventing or reducing the risk of mortality caused by a cardiovascular event in T2DM patients.

In another aspect, the present invention relates to the use of alogliptin, sitagliptin, vildagliptin, or BI 1356, or a pharmaceutically acceptable salt thereof, and optionally at least one pharmaceutically acceptable carrier for the preparation or manufacture of a medicament for preventing or reducing the risk of mortality caused by a cardiovascular event in mammals, particularly humans that have a history of CV disease, a history of hypertension, a history of hypercholesterolemia, and/or a smoking history (current/previous). Amounts of alogliptin, sitagliptin, vildagliptin, or BI 1356 for preparing or manufacturing a medicament useful for preventing or reducing the risk of mortality caused by a cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356 and optionally at least one pharmaceutically acceptable carrier are useful for preventing or reducing the risk of mortality caused by a cardiovascular event in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, and optionally at least one pharmaceutically acceptable carrier for the preparation or manufacture of a medicament for preventing or reducing the risk of non-fatal myocardial infarction and/or non-fatal stroke in mammals, particularly humans. Amounts of alogliptin, sitagliptin, vildagliptin, or BI 1356 for preparing or manufacturing a medicament useful for preventing or reducing the risk of non-fatal myocardial infarction and/or non-fatal stroke range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356 and optionally at least one pharmaceutically acceptable carrier are useful for preventing or reducing the risk of non-fatal myocardial infarction and/or non-fatal stroke in T2DM patients.

In another aspect, the present invention relates to the use of alogliptin, sitagliptin, vildagliptin, or BI 1356, or a pharmaceutically acceptable salt thereof, and optionally at least one pharmaceutically acceptable carrier for the preparation or manufacture of a medicament for preventing or reducing the risk of non-fatal myocardial infarction and/or non-fatal stroke in mammals, particularly humans that have a history of CV disease, a history of hypertension, a history of hypercholesterolemia, and/or a smoking history (current/previous). Amounts of alogliptin, sitagliptin, vildagliptin, or BI 1356 for preparing or manufacturing a medicament useful for preventing or reducing the risk of non-fatal myocardial infarction and/or non-fatal stroke range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356 and optionally at least one pharmaceutically acceptable carrier are useful for preventing or reducing the risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, and optionally at least one pharmaceutically acceptable carrier for the preparation or manufacture of a medicament for preventing or reducing the risk of mortality caused by a second cardiovascular event in mammals, particularly humans, that have survived a first cardiovascular event. Amounts of alogliptin, sitagliptin, vildagliptin, or BI 1356 for preparing or manufacturing a medicament useful for preventing or reducing the risk of mortality caused by a second cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356 and optionally at least one pharmaceutically acceptable carrier are useful for preventing or reducing the risk of a second cardiovascular event in mammals, particularly humans, that have survived a first cardiovascular event, in T2DM patients.

In another aspect, the present invention relates to the use of alogliptin, sitagliptin, vildagliptin, or BI 1356, or a pharmaceutically acceptable salt thereof, and optionally at least one pharmaceutically acceptable carrier for the preparation or manufacture of a medicament for preventing or reducing the risk of mortality caused by a third cardiovascular event in mammals, particularly humans, that have survived two cardiovascular events. Amounts of alogliptin, sitagliptin, vildagliptin, or BI 1356 for preparing or manufacturing a medicament useful for preventing or reducing the risk of mortality caused by a third cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356 and optionally at least one pharmaceutically acceptable carrier are useful for preventing or reducing the risk of a third cardiovascular event in mammals, particularly humans, that have survived two cardiovascular events, in T2DM patients.

In another aspect, the present invention relates to the use of alogliptin, sitagliptin, vildagliptin, or BI 1356, or a pharmaceutically acceptable salt thereof, and optionally at least one pharmaceutically acceptable carrier for the preparation or manufacture of a medicament for prolonging the survival time following a first cardiovascular event in mammals, particularly humans. In a further aspect, the present invention provides the use of alogliptin, sitagliptin, vildagliptin, or BI 1356, or a pharmaceutically acceptable salt thereof, and optionally at least one pharmaceutically acceptable carrier for the preparation or manufacture of a medicament for increasing the time interval between a first cardiovascular event and a second cardiovascular event in mammals, particularly humans, that have survived a first cardiovascular event. Amounts of alogliptin, sitagliptin, vildagliptin, or BI 1356 for preparing or manufacturing a medicament useful for prolonging the survival time following a first cardiovascular event or for increasing the time interval between a first cardiovascular event and a second cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention relates to the use of alogliptin, sitagliptin, vildagliptin, or BI 1356, or a pharmaceutically acceptable salt thereof, and optionally at least one pharmaceutically acceptable carrier for the preparation or manufacture of a medicament for prolonging the survival time following a second cardiovascular event in mammals, particularly humans. In a further aspect, the present invention provides the use of alogliptin, sitagliptin, vildagliptin, or BI 1356, or a pharmaceutically acceptable salt thereof, and optionally at least one pharmaceutically acceptable carrier for the preparation or manufacture of a medicament for increasing the time interval between a second cardiovascular event and a third cardiovascular event in mammals, particularly humans, that have survived a second cardiovascular event. Amounts of alogliptin, sitagliptin, vildagliptin, or BI 1356 for preparing or manufacturing a medicament useful for prolonging the survival time following a second cardiovascular event or for increasing the time interval between a second cardiovascular event and a third cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention relates to the use of alogliptin, sitagliptin, vildagliptin, or BI 1356, or a pharmaceutically acceptable salt thereof, a sulfonyl urea, and optionally at least one pharmaceutically acceptable carrier for the preparation or manufacture of a medicament for preventing or reducing the risk of all cause mortality in mammals, particularly humans. Amounts of alogliptin, sitagliptin, vildagliptin, or BI 1356, for combination with a sulfonylurea, for preparing or manufacturing a medicament that are useful for preventing or reducing the risk of all cause mortality range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a sulfonylurea, and optionally at least one pharmaceutically acceptable carrier, are useful for preventing or reducing the risk of all cause mortality in T2DM patients.

In another aspect, the present invention relates to the use of alogliptin, sitagliptin, vildagliptin, or BI 1356, or a pharmaceutically acceptable salt thereof, a sulfonyl urea, and optionally at least one pharmaceutically acceptable carrier for the preparation or manufacture of a medicament for preventing or reducing the risk of all cause mortality in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with a sulfonylurea, for preparing or manufacturing a medicament useful for preventing or reducing the risk of all cause mortality range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a sulfonylurea, and optionally at least one pharmaceutically acceptable carrier, are useful for preventing or reducing the risk of all cause mortality in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, a sulfonyl urea, and optionally at least one pharmaceutically acceptable carrier for the preparation or manufacture of a medicament for preventing or reducing the risk of mortality caused by a cardiovascular event in mammals, particularly humans. Amounts of alogliptin, sitagliptin, vildagliptin, or BI 1356, for combination with a sulfonylurea, for preparing or manufacturing a medicament useful for preventing or reducing the risk of mortality caused by a cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a sulfonylurea, and optionally at least one pharmaceutically acceptable carrier, are useful for preventing or reducing the risk of mortality caused by a cardiovascular event in T2DM patients.

In another aspect, the present invention relates to the use of alogliptin, sitagliptin, vildagliptin, or BI 1356, or a pharmaceutically acceptable salt thereof, a sulfonyl urea, and optionally at least one pharmaceutically acceptable carrier for the preparation or manufacture of a medicament for preventing or reducing the risk of mortality caused by a cardiovascular event in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with a sulfonylurea, for preparing or manufacturing a medicament useful for preventing or reducing the risk of mortality caused by a cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a sulfonylurea, and optionally at least one pharmaceutically acceptable carrier, are useful for preventing or reducing the risk of mortality caused by a cardiovascular event in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, a sulfonylurea, and optionally at least one pharmaceutically acceptable carrier for the preparation or manufacture of a medicament for preventing or reducing the risk of non-fatal myocardial infarction and/or non-fatal stroke in mammals, particularly humans. Amounts of alogliptin, sitagliptin, vildagliptin, or BI 1356, for combination with a sulfonylurea, for preparing or manufacturing a medicament useful for preventing or reducing the risk of non-fatal myocardial infarction and/or non-fatal stroke range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a sulfonylurea, and optionally at least one pharmaceutically acceptable carrier, are useful for preventing or reducing the risk of non-fatal myocardial infarction and/or non-fatal stroke in T2DM patients.

In another aspect, the present invention relates to the use of alogliptin, sitagliptin, vildagliptin, or BI 1356, or a pharmaceutically acceptable salt thereof, a sulfonylurea, and optionally at least one pharmaceutically acceptable carrier for the preparation or manufacture of a medicament for preventing or reducing the risk of non-fatal myocardial infarction and/or non-fatal stroke in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with a sulfonylurea, for preparing or manufacturing a medicament useful for preventing or reducing the risk of non-fatal myocardial infarction and/or non-fatal stroke range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a sulfonylurea, and optionally at least one pharmaceutically acceptable carrier, are useful for preventing or reducing the risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, a sulfonylurea, and optionally at least one pharmaceutically acceptable carrier, for the preparation or manufacture of a medicament for preventing or reducing the risk of mortality caused by a second cardiovascular event in mammals, particularly humans, that have survived a first cardiovascular event. Amounts of alogliptin, sitagliptin, vildagliptin, or BI 1356, for combination with a sulfonylurea, for preparing or manufacturing a medicament useful for preventing or reducing the risk of mortality caused by a second cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a sulfonylurea, and optionally at least one pharmaceutically acceptable carrier are useful for preventing or reducing the risk of a second cardiovascular event in mammals, particularly humans, that have survived a first cardiovascular event, in T2DM patients.

In another aspect, the present invention relates to the use of alogliptin, sitagliptin, vildagliptin, or BI 1356, or a pharmaceutically acceptable salt thereof, a sulfonylurea, and optionally at least one pharmaceutically acceptable carrier, for the preparation or manufacture of a medicament for preventing or reducing the risk of mortality caused by a third cardiovascular event in mammals, particularly humans, that have survived two previous cardiovascular events. Amounts of alogliptin, sitagliptin, vildagliptin, or BI 1356, for combination with a sulfonylurea, for preparing or manufacturing a medicament useful for preventing or reducing the risk of mortality caused by a third cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a sulfonylurea, and optionally at least one pharmaceutically acceptable carrier are useful for preventing or reducing the risk of a third cardiovascular event in mammals, particularly humans, that have survived two previous cardiovascular events, in T2DM patients.

In another aspect, the present invention relates to the use of a combination of alogliptin, sitagliptin, vildagliptin, or BI 1356, or a pharmaceutically acceptable salt thereof, a sulfonylurea, and optionally at least one pharmaceutically acceptable carrier for the preparation or manufacture of a medicament for prolonging the survival time following a first cardiovascular event in mammals, particularly humans. In a further aspect, the present invention provides the use of a combination of alogliptin, sitagliptin, vildagliptin, or BI 1356, or a pharmaceutically acceptable salt thereof, a sulfonylurea, and optionally at least one pharmaceutically acceptable carrier for the preparation or manufacture of a medicament for increasing the time interval between a first cardiovascular event and a second cardiovascular event in mammals, particularly humans, that have survived a first cardiovascular event. Amounts of alogliptin, sitagliptin, vildagliptin, or BI 1356, for combination with a sulfonylurea, for preparing or manufacturing a medicament useful for prolonging the survival time following a first cardiovascular event or for increasing the time interval between a first cardiovascular event and a second cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention relates to the use of a combination of alogliptin, sitagliptin, vildagliptin, or BI 1356, or a pharmaceutically acceptable salt thereof, a sulfonylurea, and optionally at least one pharmaceutically acceptable carrier for the preparation or manufacture of a medicament for prolonging the survival time following a second cardiovascular event in mammals, particularly humans. In a further aspect, the present invention provides the use of a combination of alogliptin, sitagliptin, vildagliptin, or BI 1356, or a pharmaceutically acceptable salt thereof, a sulfonylurea, and optionally at least one pharmaceutically acceptable carrier for the preparation or manufacture of a medicament for increasing the time interval between a second cardiovascular event and a third cardiovascular event in mammals, particularly humans, that have survived a second cardiovascular event. Amounts of alogliptin, sitagliptin, vildagliptin, or BI 1356, for combination with a sulfonylurea, for preparing or manufacturing a medicament useful for prolonging the survival time following a second cardiovascular event or for increasing the time interval between a second cardiovascular event and a third cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention relates to the use of alogliptin, sitagliptin, vildagliptin, or BI 1356, or a pharmaceutically acceptable salt thereof, glyburide, or a pharmaceutically acceptable salt thereof, and optionally at least one pharmaceutically acceptable carrier for the preparation or manufacture of a medicament for preventing or reducing the risk of all cause mortality in mammals, particularly humans. Amounts of alogliptin, sitagliptin, vildagliptin, or BI 1356, for combination with glyburide, for preparing or manufacturing a medicament useful for preventing or reducing the risk of all cause mortality range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Medicaments comprised of alogliptin, sitagliptin, vildagliptin, or BI 1356, glyburide, and optionally at least one pharmaceutically acceptable carrier, are useful for preventing or reducing the risk of all cause mortality in T2DM patients.

In another aspect, the present invention relates to the use of alogliptin, sitagliptin, vildagliptin, or BI 1356, or a pharmaceutically acceptable salt thereof, glyburide, or a pharmaceutically acceptable salt thereof, and optionally at least one pharmaceutically acceptable carrier for the preparation or manufacture of a medicament for preventing or reducing the risk of all cause mortality in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with glyburide, for preparing or manufacturing a medicament useful for preventing or reducing the risk of all cause mortality range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Medicaments comprised of alogliptin, sitagliptin, vildagliptin, or BI 1356, glyburide, and optionally at least one pharmaceutically acceptable carrier, are useful for preventing or reducing the risk of all cause mortality in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, glyburide, or a pharmaceutically acceptable salt thereof, and optionally at least one pharmaceutically acceptable carrier for the preparation or manufacture of a medicament for preventing or reducing the risk of mortality caused by a cardiovascular event in mammals, particularly humans. Amounts of alogliptin, sitagliptin, vildagliptin, or BI 1356, for combination with glyburide, for preparing or manufacturing a medicament useful for preventing or reducing the risk of mortality caused by a cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Medicaments comprised of alogliptin, sitagliptin, vildagliptin, or BI 1356, glyburide, and optionally at least one pharmaceutically acceptable carrier, are useful for preventing or reducing the risk of mortality caused by a cardiovascular event in T2DM patients.

In another aspect, the present invention relates to the use of alogliptin, sitagliptin, vildagliptin, or BI 1356, or a pharmaceutically acceptable salt thereof, glyburide, or a pharmaceutically acceptable salt thereof, and optionally at least one pharmaceutically acceptable carrier for the preparation or manufacture of a medicament for preventing or reducing the risk of mortality caused by a cardiovascular event in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with glyburide, for preparing or manufacturing a medicament useful for preventing or reducing the risk of mortality caused by a cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Medicaments comprised of alogliptin, sitagliptin, vildagliptin, or BI 1356, glyburide, and optionally at least one pharmaceutically acceptable carrier, are useful for preventing or reducing the risk of mortality caused by a cardiovascular event in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, glyburide, or a pharmaceutically acceptable salt thereof, and optionally at least one pharmaceutically acceptable carrier for the preparation or manufacture of a medicament for preventing or reducing the risk of non-fatal myocardial infarction and/or non-fatal stroke in mammals, particularly humans. Amounts of alogliptin, sitagliptin, vildagliptin, or BI 1356, for combination with glyburide, for preparing or manufacturing a medicament useful for preventing or reducing the risk of non-fatal myocardial infarction and/or non-fatal stroke range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Medicaments comprised of alogliptin, sitagliptin, vildagliptin, or BI 1356, glyburide, and optionally at least one pharmaceutically acceptable carrier, are useful for preventing or reducing the risk of non-fatal myocardial infarction and/or non-fatal stroke in T2DM patients.

In another aspect, the present invention relates to the use of alogliptin, sitagliptin, vildagliptin, or BI 1356, or a pharmaceutically acceptable salt thereof, glyburide, or a pharmaceutically acceptable salt thereof, and optionally at least one pharmaceutically acceptable carrier for the preparation or manufacture of a medicament for preventing or reducing the risk of non-fatal myocardial infarction and/or non-fatal stroke in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with glyburide, for preparing or manufacturing a medicament useful for preventing or reducing the risk of non-fatal myocardial infarction and/or non-fatal stroke range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Medicaments comprised of alogliptin, sitagliptin, vildagliptin, or BI 1356, glyburide, and optionally at least one pharmaceutically acceptable carrier, are useful for preventing or reducing the risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, glyburide, or a pharmaceutically acceptable salt thereof, and optionally at least one pharmaceutically acceptable carrier, for the preparation or manufacture of a medicament for preventing or reducing the risk of mortality caused by a second cardiovascular event in mammals, particularly humans, that have survived a first cardiovascular event. Amounts of alogliptin, sitagliptin, vildagliptin, or BI 1356, for combination with glyburide, for preparing or manufacturing a medicament useful for preventing or reducing the risk of mortality caused by a second cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Medicaments comprised of alogliptin, sitagliptin, vildagliptin, or BI 1356, glyburide, and optionally at least one pharmaceutically acceptable carrier are useful for preventing or reducing the risk of a second cardiovascular event in mammals, particularly humans, that have survived a first cardiovascular event, in T2DM patients.

In another aspect, the present invention relates to the use of alogliptin, sitagliptin, vildagliptin, or BI 1356, or a pharmaceutically acceptable salt thereof, glyburide, or a pharmaceutically acceptable salt thereof, and optionally at least one pharmaceutically acceptable carrier, for the preparation or manufacture of a medicament for preventing or reducing the risk of mortality caused by a third cardiovascular event in mammals, particularly humans, that have survived two previous cardiovascular events. Amounts of alogliptin, sitagliptin, vildagliptin, or BI 1356, for combination with glyburide, for preparing or manufacturing a medicament useful for preventing or reducing the risk of mortality caused by a third cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day. Medicaments comprised of alogliptin, sitagliptin, vildagliptin, or BI 1356, glyburide, and optionally at least one pharmaceutically acceptable carrier are useful for preventing or reducing the risk of a third cardiovascular event in mammals, particularly humans, that have survived two previous cardiovascular events, in T2DM patients.

In another aspect, the present invention relates to the use of a combination of alogliptin, sitagliptin, vildagliptin, or BI 1356, or a pharmaceutically acceptable salt thereof, glyburide, or a pharmaceutically acceptable salt thereof, and optionally at least one pharmaceutically acceptable carrier for the preparation or manufacture of a medicament for prolonging the survival time following a first cardiovascular event in mammals, particularly humans. In a further aspect, the present invention provides the use of a combination of alogliptin, sitagliptin, vildagliptin, or BI 1356, or a pharmaceutically acceptable salt thereof, glyburide, or a pharmaceutically acceptable salt thereof, and optionally at least one pharmaceutically acceptable carrier for the preparation or manufacture of a medicament for increasing the time interval between a first cardiovascular event and a second cardiovascular event in mammals, particularly humans, that have survived a first cardiovascular event. Amounts of alogliptin, sitagliptin, vildagliptin, or BI 1356, for combination with glyburide, for preparing or manufacturing a medicament useful for prolonging the survival time following a first cardiovascular event or for increasing the time interval between a first cardiovascular event and a second cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day.

In another aspect, the present invention relates to the use of a combination of alogliptin, sitagliptin, vildagliptin, or BI 1356, or a pharmaceutically acceptable salt thereof, glyburide, or a pharmaceutically acceptable salt thereof, and optionally at least one pharmaceutically acceptable carrier for the preparation or manufacture of a medicament for prolonging the survival time following a second cardiovascular event in mammals, particularly humans. In a further aspect, the present invention provides the use of a combination of alogliptin, sitagliptin, vildagliptin, or BI 1356, or a pharmaceutically acceptable salt thereof, glyburide, or a pharmaceutically acceptable salt thereof, and optionally at least one pharmaceutically acceptable carrier for the preparation or manufacture of a medicament for increasing the time interval between a second cardiovascular event and a third cardiovascular event in mammals, particularly humans, that have survived a second cardiovascular event. Amounts of alogliptin, sitagliptin, vildagliptin, or BI 1356, for combination with glyburide, for preparing or manufacturing a medicament useful for prolonging the survival time following a second cardiovascular event or for increasing the time interval between a second cardiovascular event and a third cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for glyburide is about 0.5 mgs/day to about 15 mgs/day.

In another aspect, the present invention relates to the use of alogliptin, sitagliptin, vildagliptin, or BI 1356, or a pharmaceutically acceptable salt thereof, a biguanide, and optionally at least one pharmaceutically acceptable carrier for the preparation or manufacture of a medicament for preventing or reducing the risk of all cause mortality in mammals, particularly humans. Amounts of alogliptin, sitagliptin, vildagliptin, or BI 1356, for combination with a biguanide, for preparing or manufacturing a medicament useful for preventing or reducing the risk of all cause mortality range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a biguanide, and optionally at least one pharmaceutically acceptable carrier, are useful for preventing or reducing the risk of all cause mortality in T2DM patients.

In another aspect, the present invention relates to the use of alogliptin, sitagliptin, vildagliptin, or BI 1356, or a pharmaceutically acceptable salt thereof, a biguanide, and optionally at least one pharmaceutically acceptable carrier for the preparation or manufacture of a medicament for preventing or reducing the risk of all cause mortality in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with a biguanide, for preparing or manufacturing a medicament useful for preventing or reducing the risk of all cause mortality range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a biguanide, and optionally at least one pharmaceutically acceptable carrier, are useful for preventing or reducing the risk of all cause mortality in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, a biguanide, and optionally at least one pharmaceutically acceptable carrier for the preparation or manufacture of a medicament for preventing or reducing the risk of mortality caused by a cardiovascular event in mammals, particularly humans. Amounts of alogliptin, sitagliptin, vildagliptin, or BI 1356, for combination with a biguanide, for preparing or manufacturing a medicament useful for preventing or reducing the risk of mortality caused by a cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a biguanide, and optionally at least one pharmaceutically acceptable carrier, are useful for preventing or reducing the risk of mortality caused by a cardiovascular event in T2DM patients.

In another aspect, the present invention relates to the use of alogliptin, sitagliptin, vildagliptin, or BI 1356, or a pharmaceutically acceptable salt thereof, a biguanide, and optionally at least one pharmaceutically acceptable carrier for the preparation or manufacture of a medicament for preventing or reducing the risk of mortality caused by a cardiovascular event in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with a biguanide, for preparing or manufacturing a medicament useful for preventing or reducing the risk of mortality caused by a cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a biguanide, and optionally at least one pharmaceutically acceptable carrier, are useful for preventing or reducing the risk of mortality caused by a cardiovascular event in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, a biguanide, and optionally at least one pharmaceutically acceptable carrier for the preparation or manufacture of a medicament for preventing or reducing the risk of non-fatal myocardial infarction and/or non-fatal stroke in mammals, particularly humans. Amounts of alogliptin, sitagliptin, vildagliptin, or BI 1356, for combination with a biguanide, for preparing or manufacturing a medicament useful for preventing or reducing the risk of non-fatal myocardial infarction and/or non-fatal stroke range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a biguanide, and optionally at least one pharmaceutically acceptable carrier, are useful for preventing or reducing the risk of non-fatal myocardial infarction and/or non-fatal stroke in T2DM patients.

In another aspect, the present invention relates to the use of alogliptin, sitagliptin, vildagliptin, or BI 1356, or a pharmaceutically acceptable salt thereof, a biguanide, and optionally at least one pharmaceutically acceptable carrier for the preparation or manufacture of a medicament for preventing or reducing the risk of non-fatal myocardial infarction and/or non-fatal stroke in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with a biguanide, for preparing or manufacturing a medicament useful for preventing or reducing the risk of non-fatal myocardial infarction and/or non-fatal stroke range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a biguanide, and optionally at least one pharmaceutically acceptable carrier, are useful for preventing or reducing the risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, a biguanide, and optionally at least one pharmaceutically acceptable carrier, for the preparation or manufacture of a medicament for preventing or reducing the risk of mortality caused by a second cardiovascular event in mammals, particularly humans, that have survived a first cardiovascular event. Amounts of alogliptin, sitagliptin, vildagliptin, or BI 1356, for combination with a biguanide, for preparing or manufacturing a medicament useful for preventing or reducing the risk of mortality caused by a second cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a biguanide, and optionally at least one pharmaceutically acceptable carrier are useful for preventing or reducing the risk of a second cardiovascular event in mammals, particularly humans, that have survived a first cardiovascular event, in T2DM patients.

In another aspect, the present invention relates to the use of alogliptin, sitagliptin, vildagliptin, or BI 1356, or a pharmaceutically acceptable salt thereof, a biguanide, and optionally at least one pharmaceutically acceptable carrier, for the preparation or manufacture of a medicament for preventing or reducing the risk of mortality caused by a third cardiovascular event in mammals, particularly humans, that have survived two previous cardiovascular events. Amounts of alogliptin, sitagliptin, vildagliptin, or BI 1356, for combination with a biguanide, for preparing or manufacturing a medicament useful for preventing or reducing the risk of mortality caused by a third cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a biguanide, and optionally at least one pharmaceutically acceptable carrier are useful for preventing or reducing the risk of a third cardiovascular event in mammals, particularly humans, that have survived two previous cardiovascular events, in T2DM patients.

In another aspect, the present invention relates to the use of a combination of alogliptin, sitagliptin, vildagliptin, or BI 1356, or a pharmaceutically acceptable salt thereof, a biguanide, and optionally at least one pharmaceutically acceptable carrier for the preparation or manufacture of a medicament for prolonging the survival time following a first cardiovascular event in mammals, particularly humans. In a further aspect, the present invention provides the use of a combination of alogliptin, sitagliptin, vildagliptin, or BI 1356, or a pharmaceutically acceptable salt thereof, a biguanide, and optionally at least one pharmaceutically acceptable carrier for the preparation or manufacture of a medicament for increasing the time interval between a first cardiovascular event and a second cardiovascular event in mammals, particularly humans, that have survived a first cardiovascular event. Amounts of alogliptin, sitagliptin, vildagliptin, or BI 1356, for combination with a biguanide, for preparing or manufacturing a medicament useful for prolonging the survival time following a first cardiovascular event or for increasing the time interval between a first cardiovascular event and a second cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention relates to the use of a combination of alogliptin, sitagliptin, vildagliptin, or BI 1356, or a pharmaceutically acceptable salt thereof, a biguanide, and optionally at least one pharmaceutically acceptable carrier for the preparation or manufacture of a medicament for prolonging the survival time following a second cardiovascular event in mammals, particularly humans. In a further aspect, the present invention provides the use of a combination of alogliptin, sitagliptin, vildagliptin, or BI 1356, or a pharmaceutically acceptable salt thereof, a biguanide, and optionally at least one pharmaceutically acceptable carrier for the preparation or manufacture of a medicament for increasing the time interval between a second cardiovascular event and a third cardiovascular event in mammals, particularly humans, that have survived a second cardiovascular event. Amounts of alogliptin, sitagliptin, vildagliptin, or BI 1356, for combination with a biguanide, for preparing or manufacturing a medicament useful for prolonging the survival time following a second cardiovascular event or for increasing the time interval between a second cardiovascular event and a third cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention relates to the use of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier for the preparation or manufacture of a medicament for preventing or reducing the risk of all cause mortality in mammals, particularly humans. Amounts of alogliptin, sitagliptin, vildagliptin, or BI 1356, for combination with metformin, for preparing or manufacturing a medicament useful for preventing or reducing the risk of all cause mortality range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Medicaments comprised of alogliptin, sitagliptin, vildagliptin, or BI 1356, metformin, and optionally at least one pharmaceutically acceptable carrier, are useful for preventing or reducing the risk of all cause mortality in T2DM patients.

In another aspect, the present invention relates to the use of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier for the preparation or manufacture of a medicament for preventing or reducing the risk of all cause mortality in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with metformin, for preparing or manufacturing a medicament useful for preventing or reducing the risk of all cause mortality range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Medicaments comprised of alogliptin, sitagliptin, vildagliptin, or BI 1356, metformin, and optionally at least one pharmaceutically acceptable carrier, are useful for preventing or reducing the risk of all cause mortality in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier for the preparation or manufacture of a medicament for preventing or reducing the risk of mortality caused by a cardiovascular event in mammals, particularly humans. Amounts of alogliptin, sitagliptin, vildagliptin, or BI 1356, for combination with metformin, for preparing or manufacturing a medicament useful for preventing or reducing the risk of mortality caused by a cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Medicaments comprised of alogliptin, sitagliptin, vildagliptin, or BI 1356, metformin, and optionally at least one pharmaceutically acceptable carrier, are useful for preventing or reducing the risk of mortality caused by a cardiovascular event in T2DM patients.

In another aspect, the present invention relates to the use of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier for the preparation or manufacture of a medicament for preventing or reducing the risk of mortality caused by a cardiovascular event in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with metformin, for preparing or manufacturing a medicament useful for preventing or reducing the risk of mortality caused by a cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Medicaments comprised of alogliptin, sitagliptin, vildagliptin, or BI 1356, metformin, and optionally at least one pharmaceutically acceptable carrier, are useful for preventing or reducing the risk of mortality caused by a cardiovascular event in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier for the preparation or manufacture of a medicament for preventing or reducing the risk of non-fatal myocardial infarction and/or non-fatal stroke in mammals, particularly humans. Amounts of alogliptin, sitagliptin, vildagliptin, or BI 1356, for combination with metformin, for preparing or manufacturing a medicament useful for preventing or reducing the risk of non-fatal myocardial infarction and/or non-fatal stroke range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Medicaments comprised of alogliptin, sitagliptin, vildagliptin, or BI 1356, metformin, and optionally at least one pharmaceutically acceptable carrier, are useful for preventing or reducing the risk of non-fatal myocardial infarction and/or non-fatal stroke in T2DM patients.

In another aspect, the present invention relates to the use of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier for the preparation or manufacture of a medicament for preventing or reducing the risk of non-fatal myocardial infarction and/or non-fatal stroke in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with metformin, for preparing or manufacturing a medicament useful for preventing or reducing the risk of non-fatal myocardial infarction and/or non-fatal stroke range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Medicaments comprised of alogliptin, sitagliptin, vildagliptin, or BI 1356, metformin, and optionally at least one pharmaceutically acceptable carrier, are useful for preventing or reducing the risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier, for the preparation or manufacture of a medicament for preventing or reducing the risk of mortality caused by a second cardiovascular event in mammals, particularly humans, that have survived a first cardiovascular event. Amounts of alogliptin, sitagliptin, vildagliptin, or BI 1356, for combination with metformin, for preparing or manufacturing a medicament useful for preventing or reducing the risk of mortality caused by a second cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Medicaments comprised of alogliptin, sitagliptin, vildagliptin, or BI 1356, metformin, and optionally at least one pharmaceutically acceptable carrier are useful for preventing or reducing the risk of a second cardiovascular event in mammals, particularly humans, that have survived a first cardiovascular event, in T2DM patients.

In another aspect, the present invention relates to the use of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier, for the preparation or manufacture of a medicament for preventing or reducing the risk of mortality caused by a third cardiovascular event in mammals, particularly humans, that have survived two previous cardiovascular events. Amounts of alogliptin, sitagliptin, vildagliptin, or BI 1356, for combination with metformin, for preparing or manufacturing a medicament useful for preventing or reducing the risk of mortality caused by a third cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl. Medicaments comprised of alogliptin, sitagliptin, vildagliptin, or BI 1356, metformin, and optionally at least one pharmaceutically acceptable carrier are useful for preventing or reducing the risk of a third cardiovascular event in mammals, particularly humans, that have survived two previous cardiovascular events, in T2DM patients.

In another aspect, the present invention relates to the use of a combination of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier for the preparation or manufacture of a medicament for prolonging the survival time following a first cardiovascular event in mammals, particularly humans. In a further aspect, the present invention provides the use of a combination of alogliptin, sitagliptin, vildagliptin, or BI 1356, or a pharmaceutically acceptable salt thereof, metformin, and optionally at least one pharmaceutically acceptable carrier for the preparation or manufacture of a medicament for increasing the time interval between a first cardiovascular event and a second cardiovascular event in mammals, particularly humans, that have survived a first cardiovascular event. Amounts of alogliptin, sitagliptin, vildagliptin, or BI 1356, for combination with metformin, for preparing or manufacturing a medicament useful for prolonging the survival time following a first cardiovascular event or for increasing the time interval between a first cardiovascular event and a second cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl.

In another aspect, the present invention relates to the use of a combination of alogliptin, sitagliptin, vildagliptin, or BI 1356, or a pharmaceutically acceptable salt thereof, metformin, or a pharmaceutically acceptable salt thereof, and optionally at least one pharmaceutically acceptable carrier for the preparation or manufacture of a medicament for prolonging the survival time following a second cardiovascular event in mammals, particularly humans. In a further aspect, the present invention provides the use of a combination of alogliptin, sitagliptin, vildagliptin, or BI 1356, or a pharmaceutically acceptable salt thereof, meformin, and optionally at least one pharmaceutically acceptable carrier for the preparation or manufacture of a medicament for increasing the time interval between a second cardiovascular event and a third cardiovascular event in mammals, particularly humans, that have survived a second cardiovascular event. Amounts of alogliptin, sitagliptin, vildagliptin, or BI 1356, for combination with metformin, for preparing or manufacturing a medicament useful for prolonging the survival time following a second cardiovascular event or for increasing the time interval between a second cardiovascular event and a third cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for metformin is about 100 mgs/day to about 2500 mgs/day. The preferred pharmaceutically acceptable salt for metformin is HCl.

In another aspect, the present invention relates to the use of alogliptin, sitagliptin, vildagliptin, or BI 1356, or a pharmaceutically acceptable salt thereof, a thiazolidinedione, and optionally at least one pharmaceutically acceptable carrier for the preparation or manufacture of a medicament for preventing or reducing the risk of all cause mortality in mammals, particularly humans. Amounts of alogliptin, sitagliptin, vildagliptin, or BI 1356, for combination with a thiazolidinedione, for preparing or manufacturing a medicament useful for preventing or reducing the risk of all cause mortality range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a thiazolidinedione, and optionally at least one pharmaceutically acceptable carrier, are useful for preventing or reducing the risk of all cause mortality in T2DM patients.

In another aspect, the present invention relates to the use of alogliptin, sitagliptin, vildagliptin, or BI 1356, or a pharmaceutically acceptable salt thereof, a thiazolidinedione, and optionally at least one pharmaceutically acceptable carrier for the preparation or manufacture of a medicament for preventing or reducing the risk of all cause mortality in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with a thiazolidinedione, for preparing or manufacturing a medicament useful for preventing or reducing the risk of all cause mortality range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a thiazolidinedione, and optionally at least one pharmaceutically acceptable carrier, are useful for preventing or reducing the risk of all cause mortality in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, a thiazolidinedione, and optionally at least one pharmaceutically acceptable carrier for the preparation or manufacture of a medicament for preventing or reducing the risk of mortality caused by a cardiovascular event in mammals, particularly humans. Amounts of alogliptin, sitagliptin, vildagliptin, or BI 1356, for combination with a thiazolidinedione, for preparing or manufacturing a medicament useful for preventing or reducing the risk of mortality caused by a cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a thiazolidinedione, and optionally at least one pharmaceutically acceptable carrier, are useful for preventing or reducing the risk of mortality caused by a cardiovascular event in T2DM patients.

In another aspect, the present invention relates to the use of alogliptin, sitagliptin, vildagliptin, or BI 1356, or a pharmaceutically acceptable salt thereof, a thiazolidinedione, and optionally at least one pharmaceutically acceptable carrier for the preparation or manufacture of a medicament for preventing or reducing the risk of mortality caused by a cardiovascular event in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with a thiazolidinedione, for preparing or manufacturing a medicament useful for preventing or reducing the risk of mortality caused by a cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a thiazolidinedione, and optionally at least one pharmaceutically acceptable carrier, are useful for preventing or reducing the risk of mortality caused by a cardiovascular event in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, a thiazolidinedione, and optionally at least one pharmaceutically acceptable carrier for the preparation or manufacture of a medicament for preventing or reducing the risk of non-fatal myocardial infarction and/or non-fatal stroke in mammals, particularly humans. Amounts of alogliptin, sitagliptin, vildagliptin, or BI 1356, for combination with a thiazolidinedione, for preparing or manufacturing a medicament useful for preventing or reducing the risk of non-fatal myocardial infarction and/or non-fatal stroke range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a thiazolidinedione, and optionally at least one pharmaceutically acceptable carrier, are useful for preventing or reducing the risk of non-fatal myocardial infarction and/or non-fatal stroke in T2DM patients.

In another aspect, the present invention relates to the use of alogliptin, sitagliptin, vildagliptin, or BI 1356, or a pharmaceutically acceptable salt thereof, a thiazolidinedione, and optionally at least one pharmaceutically acceptable carrier for the preparation or manufacture of a medicament for preventing or reducing the risk of non-fatal myocardial infarction and/or non-fatal stroke in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with a thiazolidinedione, for preparing or manufacturing a medicament useful for preventing or reducing the risk of non-fatal myocardial infarction and/or non-fatal stroke range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a thiazolidinedione, and optionally at least one pharmaceutically acceptable carrier, are useful for preventing or reducing the risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, a thiazolidinedione, and optionally at least one pharmaceutically acceptable carrier, for the preparation or manufacture of a medicament for preventing or reducing the risk of mortality caused by a second cardiovascular event in mammals, particularly humans, that have survived a first cardiovascular event. Amounts of alogliptin, sitagliptin, vildagliptin, or BI 1356, for combination with a thiazolidinedione, for preparing or manufacturing a medicament useful for preventing or reducing the risk of mortality caused by a second cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a thiazolidinedione, and optionally at least one pharmaceutically acceptable carrier are useful for preventing or reducing the risk of a second cardiovascular event in mammals, particularly humans, that have survived a first cardiovascular event, in T2DM patients.

In another aspect, the present invention relates to the use of alogliptin, sitagliptin, vildagliptin, or BI 1356, or a pharmaceutically acceptable salt thereof, a thiazolidinedione, and optionally at least one pharmaceutically acceptable carrier, for the preparation or manufacture of a medicament for preventing or reducing the risk of mortality caused by a third cardiovascular event in mammals, particularly humans, that have survived two previous cardiovascular events. Amounts of alogliptin, sitagliptin, vildagliptin, or BI 1356, for combination with a thiazolidinedione, for preparing or manufacturing a medicament useful for preventing or reducing the risk of mortality caused by a third cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). Medicaments comprised of alogliptin, sitagliptin, vildagliptin, or BI 1356, a thiazolidinedione, and optionally at least one pharmaceutically acceptable carrier are useful for preventing or reducing the risk of a second cardiovascular event in mammals, particularly humans, that have survived two previous cardiovascular events, in T2DM patients.

In another aspect, the present invention relates to the use of a combination of alogliptin, sitagliptin, vildagliptin, or BI 1356, or a pharmaceutically acceptable salt thereof, a thiazolidinedione, and optionally at least one pharmaceutically acceptable carrier for the preparation or manufacture of a medicament for prolonging the survival time following a first cardiovascular event in mammals, particularly humans. In a further aspect, the present invention provides the use of a combination of alogliptin, sitagliptin, vildagliptin, or BI 1356, or a pharmaceutically acceptable salt thereof, a thiazolidinedione, and optionally at least one pharmaceutically acceptable carrier for the preparation or manufacture of a medicament for increasing the time interval between a first cardiovascular event and a second cardiovascular event in mammals, particularly humans, that have survived a first cardiovascular event. Amounts of alogliptin, sitagliptin, vildagliptin, or BI 1356, for combination with a thiazolidinedione, for preparing or manufacturing a medicament useful for prolonging the survival time following a first cardiovascular event or for increasing the time interval between a first cardiovascular event and a second cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention relates to the use of a combination of alogliptin, sitagliptin, vildagliptin, or BI 1356, or a pharmaceutically acceptable salt thereof, a thiazolidinedione, and optionally at least one pharmaceutically acceptable carrier for the preparation or manufacture of a medicament for prolonging the survival time following a second cardiovascular event in mammals, particularly humans. In a further aspect, the present invention provides the use of a combination of alogliptin, sitagliptin, vildagliptin, or BI 1356, or a pharmaceutically acceptable salt thereof, a thiazolidinedione, and optionally at least one pharmaceutically acceptable carrier for the preparation or manufacture of a medicament for increasing the time interval between a second cardiovascular event and a third cardiovascular event in mammals, particularly humans, that have survived a second cardiovascular event. Amounts of alogliptin, sitagliptin, vildagliptin, or BI 1356, for combination with a thiazolidinedione, for preparing or manufacturing a medicament useful for prolonging the survival time following a second cardiovascular event or for increasing the time interval between a second cardiovascular event and a third cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd).

In another aspect, the present invention relates to the use of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier for the preparation or manufacture of a medicament for preventing or reducing the risk of all cause mortality in mammals, particularly humans. Amounts of alogliptin, sitagliptin, vildagliptin, or BI 1356, for combination with rosiglitazone or pioglitazone, for preparing or manufacturing a medicament useful for preventing or reducing the risk of all cause mortality range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of alogliptin, sitagliptin, vildagliptin, or BI 1356, and rosiglitazone or pioglitazone, and optionally at least one pharmaceutically acceptable carrier, are useful for preventing or reducing the risk of all cause mortality in T2DM patients.

In another aspect, the present invention relates to the use of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier for the preparation or manufacture of a medicament for preventing or reducing the risk of all cause mortality in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with rosiglitazone or pioglitazone, for preparing or manufacturing a medicament useful for preventing or reducing the risk of all cause mortality range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of alogliptin, sitagliptin, vildagliptin, or BI 1356, and rosiglitazone or pioglitazone, and optionally at least one pharmaceutically acceptable carrier, are useful for preventing or reducing the risk of all cause mortality in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier for the preparation or manufacture of a medicament for preventing or reducing the risk of mortality caused by a cardiovascular event in mammals, particularly humans. Amounts of alogliptin, sitagliptin, vildagliptin, or BI 1356, for combination with rosiglitazone or pioglitazone, for preparing or manufacturing a medicament useful for preventing or reducing the risk of mortality caused by a cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of alogliptin, sitagliptin, vildagliptin, or BI 1356, and rosiglitazone or pioglitazone, and optionally at least one pharmaceutically acceptable carrier, are useful for preventing or reducing the risk of mortality caused by a cardiovascular event in T2DM patients.

In another aspect, the present invention relates to the use of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier for the preparation or manufacture of a medicament for preventing or reducing the risk of mortality caused by a cardiovascular event in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with rosiglitazone or pioglitazone, for preparing or manufacturing a medicament useful for preventing or reducing the risk of mortality caused by a cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of alogliptin, sitagliptin, vildagliptin, or BI 1356, and rosiglitazone or pioglitazone, and optionally at least one pharmaceutically acceptable carrier, are useful for preventing or reducing the risk of mortality caused by a cardiovascular event in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier for the preparation or manufacture of a medicament for preventing or reducing the risk of non-fatal myocardial infarction and/or non-fatal stroke in mammals, particularly humans. Amounts of alogliptin, sitagliptin, vildagliptin, or BI 1356, for combination with rosiglitazone or pioglitazone, for preparing or manufacturing a medicament useful for preventing or reducing the risk of non-fatal myocardial infarction and/or non-fatal stroke range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of alogliptin, sitagliptin, vildagliptin, or BI 1356, and rosiglitazone or pioglitazone, and optionally at least one pharmaceutically acceptable carrier, are useful for preventing or reducing the risk of non-fatal myocardial infarction and/or non-fatal stroke in T2DM patients.

In another aspect, the present invention relates to the use of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier for the preparation or manufacture of a medicament for preventing or reducing the risk of non-fatal myocardial infarction and/or non-fatal stroke in mammals, particularly humans that have a history of CV disease, a history of hypertension, a history of hypercholesterolemia, and/or a smoking history (current/previous). Amounts of alogliptin, sitagliptin, vildagliptin, or BI 1356, for combination with rosiglitazone or pioglitazone, for preparing or manufacturing a medicament useful for preventing or reducing the risk of non-fatal myocardial infarction and/or non-fatal stroke range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of alogliptin, sitagliptin, vildagliptin, or BI 1356, and rosiglitazone or pioglitazone, and optionally at least one pharmaceutically acceptable carrier, are useful for preventing or reducing the risk of non-fatal myocardial infarction and/or non-fatal stroke in T2DM patients that have a history of CV disease, a history of hypertension, a history of hypercholesterolemia, and/or a smoking history (current/previous).

In another aspect, the present invention relates to the use of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier, for the preparation or manufacture of a medicament for preventing or reducing the risk of mortality caused by a second cardiovascular event in mammals, particularly humans, that have survived a first cardiovascular event. Amounts of alogliptin, sitagliptin, vildagliptin, or BI 1356, for combination with rosiglitazone or pioglitazone, for preparing or manufacturing a medicament useful for preventing or reducing the risk of mortality caused by a second cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of alogliptin, sitagliptin, vildagliptin, or BI 1356, and rosiglitazone or pioglitazone, and optionally at least one pharmaceutically acceptable carrier are useful for preventing or reducing the risk of a second cardiovascular event in mammals, particularly humans, that have survived a first cardiovascular event, in T2DM patients.

In another aspect, the present invention relates to the use of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier, for the preparation or manufacture of a medicament for preventing or reducing the risk of mortality caused by a third cardiovascular event in mammals, particularly humans, that have survived two previous cardiovascular events. Amounts of alogliptin, sitagliptin, vildagliptin, or BI 1356, for combination with rosiglitazone or pioglitazone, for preparing or manufacturing a medicament useful for preventing or reducing the risk of mortality caused by a third cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl. Medicaments comprised of alogliptin, sitagliptin, vildagliptin, or BI 1356, and rosiglitazone or pioglitazone, and optionally at least one pharmaceutically acceptable carrier are useful for preventing or reducing the risk of a third cardiovascular event in mammals, particularly humans, that have survived two previous cardiovascular events, in T2DM patients.

In another aspect, the present invention relates to the use of a combination of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier for the preparation or manufacture of a medicament for prolonging the survival time following a first cardiovascular event in mammals, particularly humans. In a further aspect, the present invention provides the use of a combination of alogliptin, sitagliptin, vildagliptin, or BI 1356, or a pharmaceutically acceptable salt thereof, rosiglitazone or pioglitazone, and optionally at least one pharmaceutically acceptable carrier for the preparation or manufacture of a medicament for increasing the time interval between a first cardiovascular event and a second cardiovascular event in mammals, particularly humans, that have survived a first cardiovascular event. Amounts of alogliptin, sitagliptin, vildagliptin, or BI 1356, for combination with rosiglitazone or pioglitazone, for preparing or manufacturing a medicament useful for prolonging the survival time following a first cardiovascular event or for increasing the time interval between a first cardiovascular event and a second cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl.

In another aspect, the present invention relates to the use of a combination of alogliptin, sitagliptin, vildagliptin, or BI 1356, or a pharmaceutically acceptable salt thereof, rosiglitazone or pioglitazone, or a pharmaceutically acceptable salt thereof, and optionally at least one pharmaceutically acceptable carrier for the preparation or manufacture of a medicament for prolonging the survival time following a second cardiovascular event in mammals, particularly humans. In a further aspect, the present invention provides the use of a combination of alogliptin, sitagliptin, vildagliptin, or BI 1356, or a pharmaceutically acceptable salt thereof, rosiglitazone or pioglitazone, and optionally at least one pharmaceutically acceptable carrier for the preparation or manufacture of a medicament for increasing the time interval between a second cardiovascular event and a third cardiovascular event in mammals, particularly humans, that have survived a second cardiovascular event. Amounts of alogliptin, sitagliptin, vildagliptin, or BI 1356, for combination with rosiglitazone or pioglitazone, for preparing or manufacturing a medicament useful for prolonging the survival time following a second cardiovascular event or for increasing the time interval between a second cardiovascular event and a third cardiovascular event range from about 0.5 mgs/day to about 400 mgs/day. Preferred alogliptin, sitagliptin, vildagliptin, or BI 1356 doses are 25 mgs/day to 100 mgs/day given once daily or in divided doses including, for example, 25 mgs/day (qd), 25 mgs/day (bid), 50 mgs/day (qd), 50 mgs/day (bid), and 100 mgs/day (qd). The preferred dosing range for rosiglitazone is about 0.5 mgs/day to about 50 mgs/day. The preferred dosing range for pioglitazone is about 0.5 mgs/day to about 100 mgs/day. The preferred pharmaceutically acceptable salt for rosiglitazone is maleate and the preferred pharmaceutically acceptable salt for pioglitazone is HCl.

The present invention includes within its scope methods for reducing the risk of mortality in a mammal by administering a DPPIV inhibitor including, but not limited to, saxagliptin, alogliptin, sitagliptin, vildagliptin, or BI 1356A, preferrably saxagliptin, sitagliptin, or vildagliptin, most preferrably saxagliptin, in combination with a further pharmaceutical agent, such as: SGLT2 inhibitors; anti-obesity agents; anti-diabetic agents; appetite suppressants; cholesterol/lipid-lowering agents; HDL-raising agents; cognition enhancing agents; agents used to treat neurodegeneration; agents used to treat respiratory conditions; agents used to treat bowel disorders; anti-inflammatory agents; anti-anxiety agents; anti-depressants; anti-hypertensive agents; cardiac glycosides; anti-tumor agents; pro-angiogenic agents, pro-regenerative agents, anti-platelet agents; PCSK9 inhibitors; transplant agents such as CTLA-4 blocking agents (Abatacept and Belatacept) and Calcineurin; bone marrow stem cell agents that increase the production of stem cells such as Erythropoietin, Erythropoietin derivatives, BNP (Nesiritide), vascular endothelial growth factor (VEGF) agonists, transforming growth factor-beta agonists, and Leukemia Inhibitory Factor agonists; Cox-2 inhibitors; Cox2/Cox-1 inhibitors; agents which cause bone marrow stem cell efflux from the bone marrow such as G-CSF, CXCR4 blocking agents (Plerixafor (rINN and USAN, also known as MOZOBIL, JM 3100 and AMD3100), and Parathyroid Hormone; agents that cause homing and proliferation and differentiation of endothelial progenitor cells such as SDF-1 by giving the agent or with ex-vivo harvest followed by giving transgenic expression; agents that increase wound healing such as PDGF agonists (REGRANEX Gel); vascular active drugs acting on the NO system; Matrix Metaloprotein inhibitors such as Promogran; bone marrow expansion drugs such as G-CSF, M-CSF, Erythropoietin, and GM-C SF; HIV drugs such as Atripla (efavirenz+tenofovir+emtricitabine); Nucleoside/Nucleotide Reverse Transcriptase Inhibitors (NRTIs); immune-based agents such as Immunitin (HE2000, alpha-epibromide), Proleukin (aldesleukin, Interleukin-2, IL-2), Remune (HIV-1 Immunogen, Salk vaccine), BAY 50-4798, and IR103; Pharmacokinetic Enhancers such as Norvir (ritonavir, RTV); Cobicistat (GS-9350); and SPI-452; Non-Nucleoside Reverse Transcriptase Inhibitors (NNRTIs) such as Intelence (etravirine, ETV, TMC-125), Rescriptor (delavirdine, DLV), Sustiva (Stocrin, efavirenz, EFV), Viramune (nevirapine, NVP), and Rilpivirine (TMC-278); Protease Inhibitors (PIs); Entry Inhibitors (including Fusion Inhibitors) such as Fuzeon (enfuvirtide, ENF, T-20), Selzentry (Celsentri, maraviroc, UK-427,857), Vicriviroc (SCH-417690, SCH-D), Ibalizumab (TNX-355), and PRO140; Integrase Inhibitors such as Isentress (raltegravir, MK-0518), GSK-572, and Elvitegravir (GS-9137); Maturation Inhibitors such as Bevirimat (PA-457); and/or Cellular Inhibitors such as Droxia or Hydrea (hydroxyurea, HU).

Examples of suitable NRTIs for use in combination with the DPPIV inhibitors include, but are not limited to, Combivir (zidovudine+lamivudine, AZT+3TC), Emtriva (emtricitabine, FTC), Epivir (lamivudine, 3TC), Epzicom (Kivexa, abacavir+lamivudine, ABC+3TC), Retrovir (zidovudine, AZT, ZDV), Trizivir (abacavir+zidovudine+lamivudine, ABC+AZT+3TC), Truvada (tenofovir DF+emtricitabine, TDF+FTC), Videx & Videx EC (didanosine, ddI), Viread (tenofovir disoproxil fumarate, TDF), Zerit (stavudine, d4T), Ziagen (abacavir, ABC), Racivir (RCV), Amdoxovir (AMDX, DAPD), Apricitabine (SPD754, AVX754), and Elvucitabine (ACH-126,443, Beta-L-Fd4C).

Protease inhibitors suitable for combination with DPPIV inhibitors for reducing the risk of all cause mortality include, but are not limited to, Agenerase (amprenavir, APV), Aptivus (tipranavir, TPV), Crixivan (indinavir, IDV), Invirase (saquinavir, SQV), Kaletra (Aluvia, lopinavir/ritonavir, LPV/r), Lexiva (Telzir, fosamprenavir, FPV), Norvir (ritonavir, RTV), Prezista (darunavir, DRV), Reyataz (atazanavir, ATV), and Viracept (nelfinavir, NFV).

Examples of suitable anti-obesity agents for use in combination with the DPPIV inhibitors include melanocortin receptor (MC4R) agonists, cannabinoid receptor modulators, growth hormone secretagogue receptor (GHSR) antagonists, galanin receptor modulators, orexin antagonists, CCK agonists, GLP-1 agonists, and other Pre-proglucagon-derived peptides; NPY1 or NPY5 antagonist, NPY2 and NPY4 modulators, corticotropin releasing factor agonists, histamine receptor-3 (H3) modulators, aP2 inhibitors, PPAR gamma modulators, PPAR delta modulators, acetyl-CoA carboxylase (ACC) inhibitors, 11-β-HSD-1 inhibitors, adinopectin receptor modulators; beta 3 adrenergic agonists, such as AJ9677 (Takeda/Dainippon), L750355 (Merck), or CP331648 (Pfizer) or other known beta 3 agonists as disclosed in U.S. Pat. Nos. 5,541,204, 5,770,615, 5,491,134, 5,776,983 and 5,488,064, a thyroid receptor beta modulator, such as a thyroid receptor ligand as disclosed in WO 97/21993 (U. Cal SF), WO 99/00353 (KaroBio) and WO 00/039077 (KaroBio), a lipase inhibitor, such as orlistat or ATL-962 (Alizyme), serotonin receptor agonists, (e.g., BVT-933 (Biovitrum) or lorcaserin (Arena)), monoamine reuptake inhibitors or releasing agents, such as fenfluramine, dexfenfluramine, fluvoxamine, fluoxetine, paroxetine, sertraline, chlorphentermine, cloforex, clortermine, picilorex, sibutramine, dexamphetamine, phentermine, phenylpropanolamine or mazindol, anorectic agents such as topiramate (Johnson & Johnson), CNTF (ciliary neurotrophic factor)/AXOKINE® (Regeneron), BDNF (brain-derived neurotrophic factor), leptin and leptin receptor modulators, or cannabinoid-1 receptor inverse agonists/neutral antagonists, such as SR-141716 (Sanofi) or SLV-319 (Solvay) and DGAT inhibitors such as those described in WO 2006/134317 (A1) (Astra Zeneca), WO 2006/044775 (A2) (Bayer), WO 2006/06019020 (A1) (Sankyo), WO 2006/082010 (A1) (Roche), WO 2004/047755 (A2) (Japan Tobacco, Tularik), and WO 2005/0727401 (A2) (Amgen, Japan Tobacco).

Examples of suitable anti-diabetic agents for use in combination with the DPPIV inhibitors include: insulin secretagogues or insulin sensitizers, which may include biguanides, sulfonyl ureas, glucosidase inhibitors, aldose reductase inhibitors, PPAR γ agonists such as thiazolidinediones, PPAR α agonists (such as fibric acid derivatives), PPAR δ antagonists or agonists, PPAR α/γ dual agonists, 11-β-HSD-1 inhibitors, dipeptidyl peptidase IV (DPPIV or DP4) inhibitors including saxagliptin, vildagliptin and sitagliptin, SGLT2 inhibitors including dapagliflozin, remogliflozin, serglifozin, and AVE2268, glycogen phosphorylase inhibitors, and/or meglitinides, as well as insulin, and/or glucagon-like peptide-1 (GLP-1), GLP-1 agonist, SIRT activators (resveratrol) and/or a PTP-1B inhibitor (protein tyrosine phosphatase-1B inhibitor).

The antidiabetic agent may be an oral antihyperglycemic such as phenformin or salts thereof. Where the antidiabetic agent is phenformin, the compounds of the present invention will be employed in a weight ratio to phenformin within the range from about 0.001:1 to about 10:1, preferably from about 0.01:1 to about 5:1.

The antidiabetic agent may also preferably be a sulfonylurea such as glyburide (also known as glibenclamide), glimepiride (disclosed in U.S. Pat. No. 4,379,785), glipizide, gliclazide or chlorpropamide, other known sulfonylureas or other antihyperglycemic agents which act on the ATP-dependent channel of the beta-cells, with glyburide and glipizide being preferred, which may be administered in the same or in separate oral dosage forms. The oral antidiabetic agent may also be a glucosidase inhibitor such as acarbose (disclosed in U.S. Pat. No. 4,904,769) or miglitol (disclosed in U.S. Pat. No. 4,639,436), which may be administered in the same or in a separate oral dosage forms.

Additional pharmaceutical agents useful in the present invention for combination with DPPIV inhibitors include PPAR γ agonist such as a thiazolidinedione oral anti-diabetic agent or other insulin sensitizers (which has an insulin sensitivity effect in NIDDM patients) such as rosiglitazone (SKB), pioglitazone (Takeda), Mitsubishi's MCC-555 (disclosed in U.S. Pat. No. 5,594,016), Glaxo-Wellcome's GL-262570, englitazone (CP-68722, Pfizer) or darglitazone (CP-86325, Pfizer, isaglitazone (MIT/J&J), JTT-501 (JPNT/P&U), L-895645 (Merck), R-119702 (Sankyo/WL), NN-2344 (Dr. Reddy/NN), or YM-440 (Yamanouchi), preferably rosiglitazone and pioglitazone.

Suitable anti-hyperlipidemia agents, or agents used to treat arteriosclerosis include an HMG CoA reductase inhibitor (e.g. mevastatin and related compounds as disclosed in U.S. Pat. No. 3,983,140,) lovastatin (mevinolin) and related compounds as disclosed in U.S. Pat. No. 4,231,938, pravastatin and related compounds such as disclosed in U.S. Pat. No. 4,346,227, simvastatin and related compounds as disclosed in U.S. Pat. Nos. 4,448,784 and 4,450,171. Other HMG CoA reductase inhibitors which may be employed herein include, but are not limited to, fluvastatin, disclosed in U.S. Pat. No. 5,354,772, cerivastatin disclosed in U.S. Pat. Nos. 5,006,530 and 5,177,080, atorvastatin disclosed in U.S. Pat. Nos. 4,681,893, 5,273,995, 5,385,929 and 5,686,104, pitavastatin (Nissan/Sankyo's nisvastatin (NK-104) or itavastatin), disclosed in U.S. Pat. No. 5,011,930, Shionogi-Astra/Zeneca rosuvastatin (visastatin (ZD-4522)) disclosed in U.S. Pat. No. 5,260,440, and related statin compounds disclosed in U.S. Pat. No. 5,753,675, pyrazole analogs of mevalonolactone derivatives as disclosed in U.S. Pat. No. 4,613,610, indene analogs of mevalonolactone derivatives as disclosed in PCT application WO 86/03488, 6-[2-(substituted-pyrrol-1-yl)-alkyl)pyran-2-ones and derivatives thereof as disclosed in U.S. Pat. No. 4,647,576, Searle's SC-45355 (a 3-substituted pentanedioic acid derivative) dichloroacetate, imidazole analogs of mevalonolactone as disclosed in PCT application WO 86/07054, 3-carboxy-2-hydroxy-propane-phosphonic acid derivatives as disclosed in French Patent No. 2,596,393, 2,3-disubstituted pyrrole, furan and thiophene derivatives as disclosed in European Patent Application No. 0221025, naphthyl analogs of mevalonolactone as disclosed in U.S. Pat. No. 4,686,237, octahydronaphthalenes such as disclosed in U.S. Pat. No. 4,499,289, keto analogs of mevinolin (lovastatin) as disclosed in European Patent Application No. 0142146A2, and quinoline and pyridine derivatives disclosed in U.S. Pat. Nos. 5,506,219 and 5,691,322. In addition, phosphinic acid compounds useful in inhibiting HMG CoA reductase suitable for use herein are disclosed in GB 2205837.

The squalene synthetase inhibitors suitable for use herein include, but are not limited to, α-phosphono-sulfonates disclosed in U.S. Pat. No. 5,712,396, those disclosed by Biller et al., J. Med. Chem., 31:1869-1871 (1998) including isoprenoid (phosphinyl-methyl)phosphonates as well as other known squalene synthetase inhibitors, for example, as disclosed in U.S. Pat. No. 4,871,721 and U.S. Pat. No. 4,924,024 and in Biller, S. A. et al., Curr. Pharm. Des., 2:1-40 (1996).

In addition, other squalene synthetase inhibitors suitable for use herein include the terpenoid pyrophosphates disclosed by Ortiz de Montellano, P. et al., J. Med. Chem., 20:243-249 (1977), the farnesyl diphosphate analog A and presqualene pyrophosphate (PSQ-PP) analogs as disclosed by Corey et al., J. Am. Chem. Soc., 98:1291-1293 (1976), phosphinylphosphonates reported by McClard, R. W. et al., J. Am. Chem. Soc., 109:5544 (1987) and cyclopropanes reported by Capson, T. L., Ph.D., dissertation, Dept. Med. Chem., Univ. Utah, Abstract, Table of Contents, pp. 16, 17, 40-43, 48-51, Summary (June 1987).

Other hypolipidemic agents suitable for use herein include, but are not limited to, fibric acid derivatives, such as fenofibrate, gemfibrozil, clofibrate, bezafibrate, ciprofibrate, clinofibrate and the like, probucol, and related compounds as disclosed in U.S. Pat. No. 3,674,836, probucol and gemfibrozil being preferred, bile acid sequestrants such as cholestyramine, colestipol and DEAE-Sephadex (SECHOLEX®, Policexide) and cholestagel (Sankyo/Geltex), as well as LIPOSTABIL® (Rhone-Poulenc), EISAI® E-5050 (an N-substituted ethanolamine derivative), imanixil (HOE-402), tetrahydrolipstatin (THL), istigmastanylphos-phorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), nicotinic acid (niacin), acipimox, acifran, neomycin, p-aminosalicylic acid, aspirin, poly(diallylmethylamine) derivatives such as disclosed in U.S. Pat. No. 4,759,923, quaternary amine poly(diallyldimethylammonium chloride) and ionenes such as disclosed in U.S. Pat. No. 4,027,009, and other known serum cholesterol lowering agents.

The other hypolipidemic agent may be an ACAT inhibitor (which also has anti-atherosclerosis activity) such as disclosed in, Drugs of the Future, 24:9-15 (1999) (Avasimibe); Nicolosi et al., “The ACAT inhibitor, CI-1011 is effective in the prevention and regression of aortic fatty streak area in hamsters”, Atherosclerosis (Shannon, Irel.), 137(1):77-85 (1998); Ghiselli, G., “The pharmacological profile of FCE 27677: a novel ACAT inhibitor with potent hypolipidemic activity mediated by selective suppression of the hepatic secretion of ApoB100-containing lipoprotein”, Cardiovasc. Drug Rev., 16(1):16-30 (1998); Smith, C. et al., “RP 73163: a bioavailable alkylsulfinyl-diphenylimidazole ACAT inhibitor”, Bioorg. Med. Chem. Lett., 6(1):47-50 (1996); Krause, B. R. et al., Chapter 6: “ACAT Inhibitors: Physiologic Mechanisms for Hypolipidemic and Anti-Atherosclerotic Activities in Experimental Animals”, Inflammation: Mediators and Pathways, CRC Press, Inc., publ., Ruffolo, Jr., R. R. et al., eds., pp. 173-198 (1995); Sliskovic et al., “ACAT inhibitors: potential anti-atherosclerotic agents”, Curr. Med. Chem., 1(3):204-225 (1994); Stout et al., “Inhibitors of acyl-CoA:cholesterol O-acyl transferase (ACAT) as hypocholesterolemic agents. 6. The first water-soluble ACAT inhibitor with lipid-regulating activity. Inhibitors of acyl-CoA:cholesterol acyltransferase (ACAT). 7. Development of a series of substituted N-phenyl-N′-[(1-phenylcyclopentyl)-methyl]ureas with enhanced hypocholesterolemic activity”, Chemtracts: Org. Chem., 8(6):359-362 (1995), or TS-962 (Taisho Pharmaceutical Co. Ltd), as well as F-1394, CS-505, F-12511, HL-004, K-10085 and YIC-C8-434.

The hypolipidemic agent may be an upregulator of LDL receptor activity such as MD-700 (Taisho Pharmaceutical Co. Ltd) and LY295427 (Eli Lilly). The hypolipidemic agent may be a cholesterol absorption inhibitor preferably Schering-Plough's SCH48461 (ezetimibe) as well as those disclosed in Atherosclerosis, 115:45-63 (1995) and J. Med. Chem., 41:973 (1998).

The other lipid agent or lipid-modulating agent may be a cholesteryl transfer protein inhibitor (CETP) such as Pfizer's CP-529,414 as well as those disclosed in WO/0038722 and in EP 818448 (Bayer) and EP 992496, and Pharmacia's SC-744 and SC-795, as well as CETi-1 and JTT-705.

The hypolipidemic agent may be an ileal Na⁺/bile acid cotransporter inhibitor such as disclosed in Drugs of the Future, 24:425-430 (1999). The ATP citrate lyase inhibitor which may be employed in the combination of the invention may include, for example, those disclosed in U.S. Pat. No. 5,447,954.

The other lipid agent also includes a phytoestrogen compound such as disclosed in WO 00/30665 including isolated soy bean protein, soy protein concentrate or soy flour as well as an isoflavone such as genistein, daidzein, glycitein or equol, or phytosterols, phytostanol or tocotrienol as disclosed in WO 00/015201; a beta-lactam cholesterol absorption inhibitor such as disclosed in EP 675714; an HDL upregulator such as an LXR agonist, a PPAR α-agonist and/or an FXR agonist; an LDL catabolism promoter such as disclosed in EP 1022272; a sodium-proton exchange inhibitor such as disclosed in DE 19622222; an LDL-receptor inducer or a steroidal glycoside such as disclosed in U.S. Pat. No. 5,698,527 and GB 2304106; an anti-oxidant such as beta-carotene, ascorbic acid, α-tocopherol or retinol as disclosed in WO 94/15592 as well as Vitamin C and an antihomocysteine agent such as folic acid, a folate, Vitamin B6, Vitamin B12 and Vitamin E; isoniazid as disclosed in WO 97/35576; a cholesterol absorption inhibitor, an HMG-CoA synthase inhibitor, or a lanosterol demethylase inhibitor as disclosed in WO 97/48701; a PPAR δ agonist for treating dyslipidemia; or a sterol regulating element binding protein-I (SREBP-1) as disclosed in WO 2000/050574, for example, a sphingolipid, such as ceramide, or neutral sphingomyelenase (N-SMase) or fragment thereof. Preferred hypolipidemic agents are pravastatin, lovastatin, simvastatin, atorvastatin, fluvastatin, pitavastatin, rosuvastatin, and ezetimibe as well as niacin and/or cholestagel.

The methods of the present invention include administering DPPIV inhibitors, especially saxagliptin, in combination with anti-hypertensive agents. Examples of suitable anti-hypertensive include beta adrenergic blockers, calcium channel blockers (L-type and/or T-type; e.g., diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan and compounds disclosed in U.S. Pat. Nos. 5,612,359 and 6,043,265), Dual ET/AII antagonist (e.g., compounds disclosed in WO 00/01389), neutral endopeptidase (NEP) inhibitors, vasopepsidase inhibitors (dual NEP-ACE inhibitors) (e.g., omapatrilat and gemopatrilat), and nitrates.

Examples of suitable pro-angiogeneic agents for use in combination with the DPPIV inhibitors of the present invention include, but are not limited to, Vascular endothelial growth factor (VEGF)—A, B, C and D, platelet-derived growth factor (PDGF)—AA, AB, BB, CC and DD, Fibroblast growth factor (FGF)—1, 2 and 4, Epidermal growth factor (EGF) as well as the receptors for the VEGF, PDGF, FGF and EGF receptors.

Examples of suitable pro-regenerative agents for use in combination with the DPPIV inhibitors of the present invention include, but are not limited to, pro-angiogenic agents, Stromal derived Factor (SDF)-1alpha, c-Kit ligand, Hepatocyte Growth Factor (HGF) as well as the receptors for rSDF-1alpha, c-Ket and HGF.

Examples of suitable anti-platelet agents for use in combination with the DPPIV inhibitors of the present invention include, but are not limited to, aspirin, abciximab, clopidogrel, cilostazol, dipyridamole, defibrotide, eptifibatide, ticlopidine, prasugrel, dipyridamole, defibrotide, and tirofiban.

The methods of the present invention include combination therapies comprising DPPIV inhibitors and selective Cox-2 or nonselective Cox-2/Cox-1 inhibiting agents for preventing or reducing the risk of mortality (all cause and CV) in mammals, particularly humans. These methods prevent or reduce the risk of mortality in patients by preventing or reducing the CV liability associated with Cox-2 or Cox-1/Cox-2 inhibitors. The present invention contemplates the combination of DPPIV inhibitors including, but not limited to saxagliptin, alogliptin, sitagliptin, vildagliptin, or BI 1356A, preferrably saxagliptin, sitagliptin, and vildagliptin is preferred, most preferrably saxagliptin, with the following Cox-2 or Cox-2/Cox-1 inhibiting agents: Celebrex, Vioxx, Bextra, Aspirin, Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol), or Oxaprozin (Daypro).

The present invention contemplates the combination of DPPIV inhibitors and vascular active drugs acting on NO system for reducing mortality (all cause and CV) in mammals, particularly humans. Vascular active drugs include, but are not limited to, Resveratrol: (increase eNOS), Carbachol, Bradykinin, calcium ionophores (e.g. A23187), ACE inhibitors (already covered), NO/cGMP system drugs, cGMP-specific phosphodiesterase type 5 drugs (such as Sildenafil, Vardenafil, and Tadalafil), or ANP.

### DEFINITIONS

The term “Acute CV Events” means that a Phase 2b/3 subject was diagnosed, by a medical physician, with one or more of the following events: acute coronary syndrome, acute myocardial infarction, agonal rhythm, amaurosis fugax, angina unstable, arteriospasm coronary Balint's syndrome, basal ganglia haemorrhage, basilar artery occlusion, basilar artery stenosis, basilar artery thrombosis, brain stem haemorrhage, brain stem infarction, brain stem ischaemia, brain stem thrombosis, cardiac arrest, cardiac death, cardiogenic shock, cardio-respiratory arrest, carotid arterial embolus, carotid artery bypass, carotid artery dissection, carotid artery insufficiency, carotid artery occlusion, carotid artery stenosis, carotid artery stent insertion, carotid artery thrombosis, carotid endarterectomy, cerebellar artery occlusion, cerebellar artery thrombosis, cerebellar embolism, cerebellar haemorrhage, cerebellar infarction, cerebral artery embolism, cerebral artery occlusion, cerebral artery stenosis, cerebral artery thrombosis, cerebral circulatory failure, cerebral haemorrhage, cerebral hypoperfusion, cerebral infarction, cerebral ischaemia, cerebral thrombosis, cerebrovascular accident, cerebrovascular insufficiency, cerebrovascular stenosis, circulatory collapse, coronary angioplasty, coronary arterial stent insertion, coronary artery bypass, coronary artery dissection, coronary artery embolism, coronary artery insufficiency, coronary artery occlusion, coronary artery reocclusion, coronary artery restenosis, coronary artery stenosis, coronary artery thrombosis, coronary bypass thrombosis, coronary endarterectomy, Coronary ostial stenosis, coronary revascularisation, dissecting coronary artery aneurysm, electromechanical dissociation, embolic cerebral infarction, embolic stroke, haemorrhage coronary artery, haemorrhage intracranial, haemorrhagic cerebral infarction, haemorrhagic stroke, haemorrhagic transformation stroke, infarction, in-stent coronary artery restenosis, intraventricular haemorrhage, ischaemic cerebral infarction, ischaemic stroke, lacunar infarction, lateral medullary syndrome, myocardial infarction, myocardial ischaemia, myocardial reperfusion injury, papillary muscle infarction, percutaneous coronary intervention, post procedural myocardial infarction, post procedural stroke, postinfarction angina, precerebral artery occlusion, prinzmetal angina, putamen haemorrhage, reversible ischaemic neurological deficit, shock, silent myocardial infarction, Sneddon's syndrome, spinal artery embolism, spinal cord haemorrhage, spinal epidural haemorrhage, stroke in evolution, subarachnoid haemorrhage, subendocardial ischaemia, sudden cardiac death, sudden death, thalamic infarction, thalamus haemorrhage, thrombotic cerebral infarction, thrombotic stroke, transient ischaemic attack, vascular graft occlusion, ventricular asystole, ventricular fibrillation, ventricular flutter, ventricular tachyarrhythmia, ventricular tachycardia, vertebral artery occlusion, vertebral artery stenosis, vertebral artery thrombosis, vertebrobasilar insufficiency, and Wallenberg syndrome.

The term “All-cause Mortality or All-cause death” as used herein means death by any means and includes death by an adverse CV event.

The term “bid” as used herein means twice dailey. For example, the term 50 mg/day (bid) means that the subject received 50 mgs twice within the same day or a total of 100 mgs in one day.

The term “biguanide” as used herein means the class of anti-diabetic agents used to manage type I or type II diabetes mellitus by decreasing blood glucose levels and includes, but is not limited to, buformin, phenformin and metformin. The preferred biguanide for combination therapies encompassed by methods of the present invention is metformin.

The term “cardiovascular death or CV death” as used herein means death due to stroke (cerebrovascular accident), heart attack, heart failure, or death related to blood vessel problems not in the heart or brain.

The term “DPP-IV inhibitor or DPP-4 inhibitor” means a compound that inhibits the enzyme DPP-IV. The DPP-IV enzyme is responsible for the inactivation of endogenous glucagon-like peptide 1 (GLP-1) and glucose-dependent insulinotropic peptide (GIP) Inhibition of DPP-IV increases the active form of GLP-1 and GIP in plasma, modulating the physiological mechanism of insulin secretion and decreasing glucagon release, thereby reducing postprandial and fasting glucose levels. DPP-IV inhibitors contemplated by the methods of the present invention include, but are not limited to, alogliptin, saxagliptin, sitagliptin, vildagliptin, and the xanthine DPP-IV inhibitor BI 1356 (proposed trade name ONDERO).

The term “mixed dyslipidemia” as used herein means patients (or subjects) that have high cholesterol and have another lipid abnormality, particularly high triglycerides. The methods of the present invention are useful for treating patients or subjects with high cholesterol and high triglycerides.

The term “MACE or Primary MACE” or “major adverse cardiac event” as used herein relates to the occurrence of non-fatal myocardial infarction, non-fatal stroke, or CV death.

The term “pharmaceutically acceptable carrier” as used herein means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The present invention provides pharmaceutical compositions which comprise compounds of the present invention formulated together with one or more non-toxic pharmaceutically acceptable carriers. The present invention provides pharmaceutical compositions which comprise a DPP-IV inhibitor of the present invention formulated together with one or more non-toxic pharmaceutically acceptable carriers. The DPP-IV inhibitors of the present invention, in particular saxagliptin, may be formulated into pharmaceutical compositions as described in US 2005/0266080 and WO 05/117841, herein incorporated by reference in their entirety for any purpose.

The term “pharmaceutically acceptable salt” or “salt,” as used herein, refers to salts that are well known in the art. For example, S. M Berge et al. describe pharmaceutically acceptable salts in detail in J. Pharmaceutical Sciences, 66:1-19 (1977). Examples of pharmaceutically acceptable salts include acetic acid, aspartic acid, benzenesulfonic acid, benzoic acid, butyric acid, citric acid, fumaric acid, hydrochloric acid, hydrobromic acid, lactic acid, maleic acid, malonic acid, methanesulfonic acid, 4-methylbenzenesulfonic acid, nicotinic acid, phosphoric acid, succinic acid, sulfuric acid, or tartaric acid, prepared by using methods well known in the art. The preferred pharmaceutically acceptable salt of saxagliptin for use in the methods of the present invention is HCl.

The term “qd” as used herein means daily. For example, the term 50 mg/day (qd) means that the subject received 50 mgs a day.

The term “saxagliptin” means the compound (1S,3S,5S)-2-[(2S)-2-amino-2-(3-hydroxyadamantan-1-yl)acetyl]-2-azabicyclo[3.1.0]hexane-3-carbonitrile that can be prepared using the synthetic procedures described in U.S. Pat. No. 6,395,767, in particular Example 60. The present invention encompasses pharmaceutically acceptable salts, as defined herein, of saxagliptin, in particular the benzoic acid, fumaric acid, hydrobromic acid, hydrochloric acid, methane sulfonic acid, tartaric acid, trifluoroacetic acid, salts of saxagliptin, for use in the methods described herein. Saxagliptin salts are prepared using techniques well known in the art or by using the procedures described in U.S. Pat. Nos. 6,395,767, and 7,420,079, and WO 08/131,149, herein incorporated by reference in their entirety for any purpose. The methods of the present invention also encompass crystalline forms of saxagliptin as hydrates and/or solvates. In particular, the mono hydrate of saxagliptin is encompassed by the methods of the present invention. The clinical studies, described herein in part, were conducted with saxagliptin as the mono hydrate. Other hydrate forms are also contemplated by the present invention including the hemi hydrate or (2:1) saxagliptin:H₂O and salt form hydrates. Hydrates of saxagliptin salts are also contemplated by the methods of the present invention and include the crystalline salt/hydrates disclosed in WO 08/131,149.

The term “Secondary MACE” as used herein means major adverse cardiac events and relates to the occurrence of non-fatal MI, non-fatal stroke, CV death, and death by any means (All-cause death or All-cause Mortality).

The term “stroke” as used herein means loss of brain function(s) due to a disturbance in the blood supply to the brain. This can be due to ischemia (lack of blood supply) caused by thrombosis or embolism or due to a hemorrhage. As a result, the affected area of the brain is unable to function, leading to inability to move one or more limbs on one side of the body, inability to understand or formulate speech or inability to see one side of the visual field.

The term “sulfonylurea” as used herein means the class of anti-diabetic agents used to manage T2DM by increasing insulin release from pancreatic beta cells and includes, but is not limited to, acetohexamide, chlorpropamide, glibenclamide (glyburide), gliclazide, glimepiride, glipizide, glyclopyramide, tolazamide, and tolbutamide. The preferred sulfonylurea for combination therapies encompassed by methods of the present invention is glyburide.

The term “thiazolidinedione” as used herein means the class of anti-diabetic agents used to manage T2DM by decreasing insulin resistance and lowering blood sugar levels and includes, but is not limited to, rosiglitazone, pioglitazone, and troglitazone. The preferred thiazolidinediones for combination therapies encompassed by methods of the present invention are rosiglitazone and pioglitazone.

The methods of the present invention encompass the use of metabolites of saxagliptin prepared by metabolic processes occurring in the human or mammal body (in vivo) or processes occurring in vitro. In particular, the present invention contemplates the metabolite of saxagliptin, shown in Table 1, as useful in mammals, particularly humans, for preventing or reducing the risk of: (1) all cause mortality (2) mortality caused by a cardiovascular event; (3) non-fatal myocardial infarction (4) non-fatal stroke; and (5) mortality caused by a second cardiovascular event in subjects that have survived a first CV event.

Table 1

| | Saxagliptin | Metabolite of Saxagliptin |
|---|---|---|
| Structure | 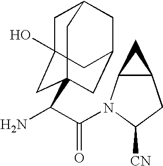 | 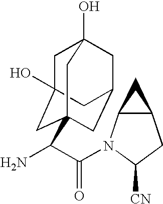 |
| Name | (1S,3S,5S)-2-[(2S)- | (1S,3S,5S)-2-[ |
| | 2-amino-2-(3- | (2S)-2-amino-2- |
| | hydroxyadamantan- | (3,5-dihydroxyadamantan- |
| | 1-yl)acetyl]-2- | 1-yl)acetyl]-2- |
| | azabicyclo[3.1.0]hexane-3- | azabicyclo[3.1.0] |
| | carbonitrile | hexane-3-carbonitrile |
| Human | Ki = 1.3 nM | Ki = 2.6 nM |
| DPP-IV | | |
| Inhibition | | |
| Preparation | US 6395767 | Biotransformation in vivo |

### Clinical Data

Saxagliptin was evaluated in a total of 5346 subjects in its Phase 1-3 clinical development, 4042 subjects received saxagliptin. In the development program, saxagliptin was studied, and well tolerated, at maximum oral daily doses of 400 mg for up to 2 weeks, 100 mg for up to 6 weeks, 40 mg and 20 mg for up to 12 weeks, and 2.5 mg, 5 mg and 10 mg for up to 2 years. These studies showed that saxagliptin, overall, did not lead to adverse CV effects related to blood pressure or heart rate.

The mortality and adverse CV event data shown in the Tables and Figures herein were analyzed from the Phase 2b and Phase 3 studies (Phase 2b/3). Table 2 shows the number of randomized and treated subjects in the Phase 2b/3 studies.

TABLE 2
Number of Randomized and Treated Subjects in the Phase 2b
and Core Phase 3 Studies

| | Saxagliptin | Saxagliptin | Saxagliptin | |
|---|---|---|---|---|
| Study | 2.5 mg | 5 mg | 10 mg | Placebo |
| Monotherapy | | | | |
| -008* | 55 | 47 | 63 | 67 |
| -011 | 102 | 106 | 98 | 95 |
| -038** | 74 | 74 | NA | 74 |
| Add-on combination | | | | |
| Saxagliptin + TZD (-013) | 195 | 186 | NA | 184 |
| Saxagliptin + SU (-040) | 248 | 253 | NA | 267 |
| Saxagliptin + MET (-014) | 192 | 191 | 181 | 179 |

| | Saxagliptin 5 mg + | Saxagliptin 10 mg + | Saxagliptin | |
|---|---|---|---|---|
| | Metformin | Metformin | 10 mg | Metformin |
| Inital Comb. | 320 | 323 | 335 | 328 |

*Includes subjects in the saxagliptin 2.5, 5, 10 mg, and placebo groups from the 0-40 mg cohort. Number of randomized and treated subjects in the saxagliptin 20, 40, 100 mg, and the placebo groups of the 0-100 mg cohort were 54, 52, 44, and 41, respectively (not shown in the table).
**Includes subjects in the saxagliptin 2.5 mg QAM, 5 mg QAM, and placebo groups. Number of randomized and treated subjects in the saxagliptin 2.5/5 mg titration group and the 5 mg PM group were 71 and 73, respectively (not shown in the table).

The data in Table 3 shows the actual number of events in the pooled analysis and that saxagliptin reduces the incidence of CV events (ACE), Inv-CV death/MI/Stroke (MACE), all death, and CV death in subjects as compared to control (placebo or metformin). For this analysis of the saxagliptin Phase 2b/3 clinical data, the primary endpoint, Major Adverse Cardiovascular Events (MACE as Investigator CV death/MI/Stoke or CEC-adjudicated CV death/MI/stroke and Cardiovascular Events (ACE; acute, clinically significant events, including cardiac revascularization procedures) were identified using selected MedDRA Preferred Terms. CV events were analyzed in the most comprehensive available dataset: 8 randomized, double blind, Phase 2b/3 trials, which included 4607 patients (3206 randomized to saxagliptin 2.5 mgs, 5 mgs, or 10 mgs 150 randomized to saxagliptin 20 mgs, 40 mgs, or 100 mgs; and 1251 randomized to placebo, metformin, or up titrated glyburide) most described in Table 2.

TABLE 3
The Frequency of Cardiovascular Events and Death

| | Saxa 2.5 mg | Saxa 5 mg | Saxa 10 mg | All Saxa^{a} | Control |
|---|---|---|---|---|---|
| | N = 937 | N = 1269 | N = 1000 | N = 3356 | N = 1251 |
| | n (%) | n (%) | n (%) | n (%) | n (%) |
| CV events | 14 (1.5) | 10 (0.8) | 14 (1.4) | 38 (1.1) | 23 (1.8) |
| Inv-CV death/MI/stroke | 6 (0.6) | 6 (0.5) | 11 (1.1) | 23 (0.7) | 18 (1.4) |
| CEC-CV death/MI/stroke^{b} | 6 (0.6) | 7 (0.6) | 9 (0.9) | 22 (0.7) | 18 (1.4) |
| All MI | 2 (0.2) | 4 (0.3) | 2 (0.2) | 8 (0.2) | 8 (0.6) |
| All stroke | 4 (0.4) | 4 (0.3) | 3 (0.3) | 11 (0.3) | 5 (0.4) |
| Other CV death | 0 | 0 | 4 (0.4) | 4 (0.1) | 6 (0.5) |
| All death | 3 (0.3) | 3 (0.2) | 4 (0.4) | 10 (0.3) | 12 (1.0) |
| CV death^{c} | 1 (0.1) | 2 (0.2) | 4 (0.4) | 7 (0.2) | 10 (0.8) |

Abbreviations: CEC, clinical events committee; CV, cardiovascular; Inv, investigator; MI, myocardial infarction.
^{a}Includes patients on 2.5, 5, and 10, as well as on 20, 40, and 100 mg/d Saxa in the Dose-Ranging trial.
^{b}The “all MI” and “all stroke” categories include patients with a fatal and/or non-fatal categorical event,
Patients with a MI and stroke were counted in each. Patients in the “other CV death” category have a CV-related death but no definite MI or stroke event.
^{c}The investigator/sponsor and CEC-adjudicated CV death assessment was identical

In the analysis of all Phase 2b/3 studies, a total of 17 CV deaths were reported: one subject (0.1%) in the saxagliptin 2.5 mg group, 2 subjects (0.2%) in the saxagliptin 5 mg group; 4 subjects (0.4%) in the saxagliptin 10 mg group; 6 subjects (0.7%) in the placebo group; and 4 subjects (1.2%) treated with metformin. There was one additional death, characterized by the investigator as unrelated to saxagliptin (death occurred 40 days after receiving a single 10 mg dose of saxagliptin), in a subject with a history of hepatic impairment (not included in the analysis). The overall frequency of CV deaths was 0.2% (7/3356) in subjects treated with saxagliptin and 0.8% (10/1251) in subjects in the comparator or control groups. For all cause mortality (death by any means), the frequency was 0.3% (10/3356) in subjects treated with saxagliptin and 1.0% (12/1251) in subjects in the control groups. This data shows that treatment with saxagliptin reduces risk of mortality (and CV mortality) in a patient population with increased mortality rates, people with T2DM.

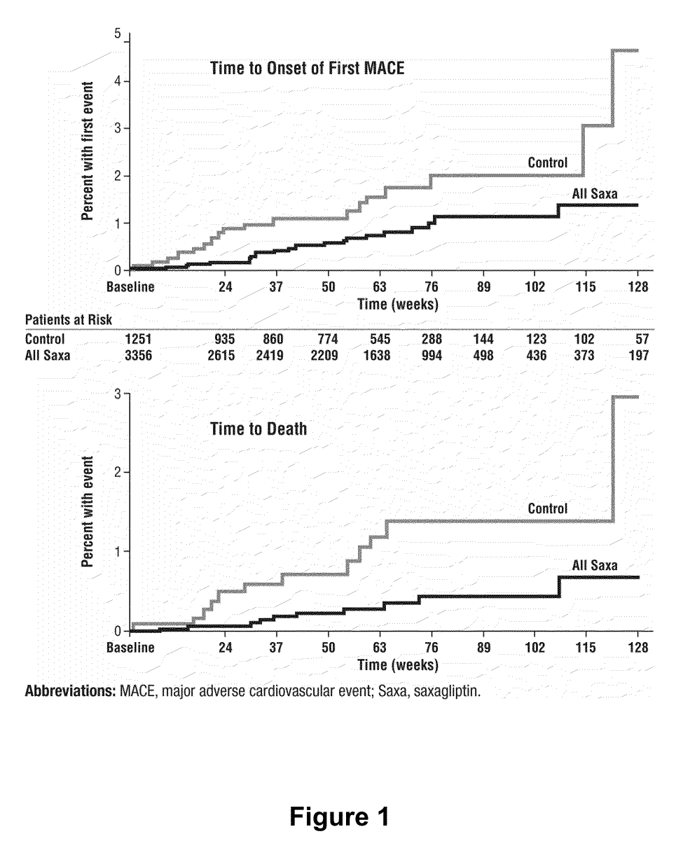

With regard to major adverse cardiovascular events (MACE or Primary MACE, CV death/MI/stroke), a total of 41 subjects in the Phase 2b/3 pooled population were identified as having a Primary MACE, 23 (0.7%) in the ALL SAXA group (FIG. 1 shows the cumulative proportion of subjects with events by Kaplan-Meier analysis) and 18 (1.4%) in the comparator or control group. Primary MACE includes subjects that experienced CV death, non-fatal myocardial infarction (MI), and non-fatal stroke. Unexpectedly, the incidence of all cause death, and MACE (CV death, non-fatal MI, and non-fatal stroke) in the Phase 2b/3 pooled population was reduced for the population administered saxagliptin, as illustrated graphically in FIG. 1.

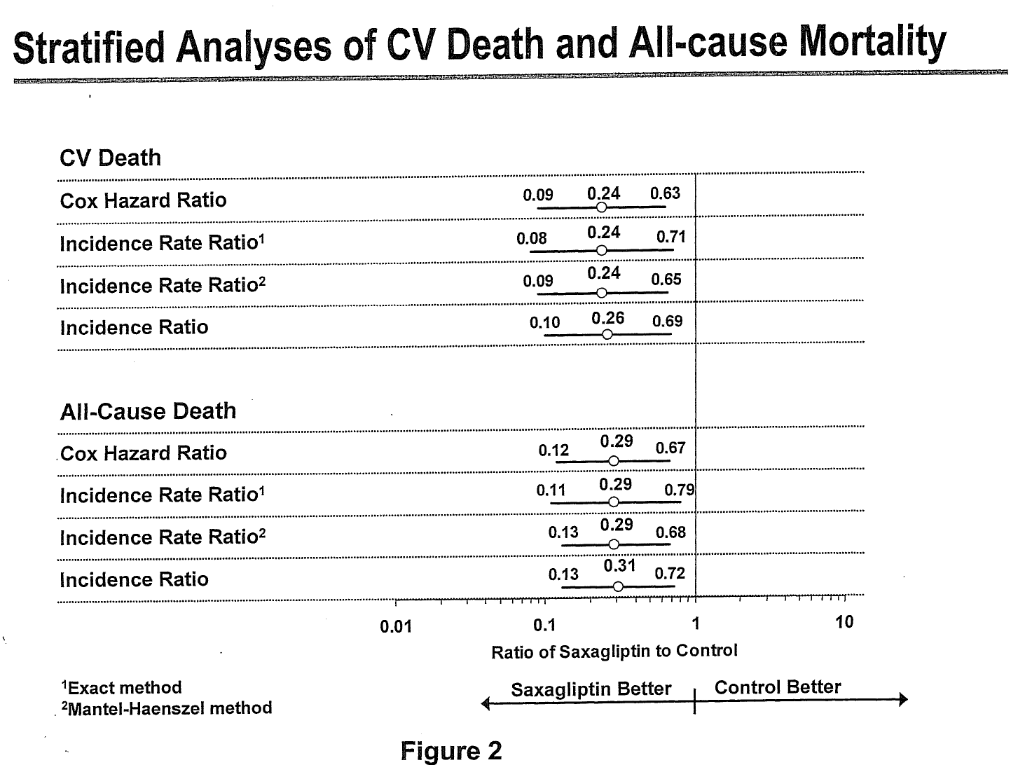

Further, analyses were conducted for CV deaths and all-cause deaths for the subject groups administered saxagliptin or placebo/metformin using an additional three different methods (Cox Hazard Ratio, Exact Method, and Mantel-Haenszel method). Mortality incidences caused by stroke, heart attack, heart failure, or blood vessel problems not associated with the heart or brain, were defined as CV death. Death by any means, including CV death, was defined as All-cause Mortality or All-cause death. Unexpectedly, the analyses of relative risk, utilizing three different methods as summarized in FIG. 2, show that the relative risk of mortality, CV and All-cause death, were reduced for subjects administered saxagliptin as compared to the control groups.

To explain FIG. 2 in more detail, the “point estimate,” represents the best estimate of the risk in the population studied (diabetics not on prior treatment, or taking stable doses of metformin, sulfonylureas, or thiazolidinediones, all with blood sugar values which suggest they are not optimally treated for diabetes). For death by any cause the “point estimate” (i.e. 0.29 for the Cox, Exact, and Mantel-Haenszel methods) means that, for an equivalent group size, during the time it would take 100 subjects in the comparator group to die, only 29 subjects in the saxagliptin group would die. For CV death, only 24 subjects administered saxagliptin would die during the time period in which 100 people, not administered saxagliptin, would suffer a CV death. The lines represent the “95% confidence interval”. In other words, there is a 95% confidence level that if the entire defined population were sampled, the risk of CV death or All-cause death, would fall within the range indicated by the line. For example, at best when 100 people on the comparator group had died only 12 subjects being treated with saxagliptin will have died and at worst, when 100 people on the comparator group had died 67 subjects on saxagliptin will have died.

Table 4 shows incidence rates for the Phase 2b/3 population represented as 1000 patient years. Acute CV events, as defined herein, includes all cardiovascular disorders associated with Phase 2b/3 subjects that were diagnosed by a medical physician during the studies as acute and clinically significant including MACE events and also procedures (for example, cardiac bypass surgery) and severe ischemic event such as unstable angina. Primary MACE means non-fatal myocardial infarction (MI), non-fatal stroke, and CV death. Secondary MACE accounts for All-cause deaths (death by any means) in addition to non-fatal MI, non-fatal stroke, and CV death. The incidence rates in subjects administered saxagliptin ranged from 6.2 to 10.7 events per 1000 patient years. Incident rates in subjects administered placebo or metformin ranged from 13.1 to 17.6 events per 1000 patient years (Table 4).

TABLE 4
Incidence Rates per 1000 Patient Years for
Acute CV Events, Primary MACE, and Secondary
MACE Phase 2/3 Pooled Population

| | | Incidence rate ± SE |
|---|---|---|
| Type of CV Event | Treatment Group | (events/1000 patient-years) |
| Acute CV Events | Saxagliptin | 10.7 ± 1.8 |
| Acute CV Events | Control | 17.6 ± 3.7 |
| Primary MACE | Saxagliptin | 6.2 ± 1.3 |
| Primary MACE | Control | 13.9 ± 3.3 |
| Secondary MACE | Saxagliptin | 7.0 ± 1.4 |
| Secondary MACE | Control | 15.4 ± 3.5 |

Further analyses were conducted on the Phase 2b/3 pooled population to evaluate the effect of saxagliptin on subjects with an increased risk of having a CV event. A total of 569 subjects from the Phase 2b/3 studies were determined to have clinically evident CV disease, defined as a history of myocardial infarction, congestive heart failure, hospitalization for unstable angina pectoris, stable angina pectoris, prior percutaneous coronary intervention, prior coronary artery bypass surgery, coronary artery disease, cerebrovascular disease, or peripheral vascular disease (Table 5). While T2DM is a well-recognized risk factor for CV events, the majority (≧80%) of subjects enrolled in the Phase 2b/3 studies had at least one additional risk factor for CV events (including prior history of hypertension, hypercholesterolemia, smoking, or first degree relative with premature coronary heart disease). Approximately 15% of the Phase 2b/3 subjects were elderly (≧65 years of age). The data in Table 5 indicate that the Phase 2b/3 program included a substantial number of subjects at increased risk for CV. Therefore, the data shown in Table 4 and FIGS. 1 and 2, viewed within the context of Table 5, provides evidence that saxagliptin reduces the risk or incidence of mortality and the risk or incidence of MACE in subjects that have previously experienced an adverse CV event.

TABLE 5
Cardiovascular Risk Factors at Baseline in the Phase 2b/3 Pooled Population

| | Saxa 2.5 mg | Saxa 5 mg | Saxa 10 mg | All Saxa(a) | Control(b) |
|---|---|---|---|---|---|
| | N = 937 | N = 1269 | N = 1000 | N = 3356 | N = 1251 |
| History of | 118 (12.6) | 150 (11.8) | 118 (11.8) | 404 (12.0) | 165 (13.2) |
| cardiovascular | | | | | |
| disease*, | | | | | |
| n (%) | | | | | |
| At least one other | 777 (82.9) | 1015 (80.0) | 803 (80.3) | 2724 (81.2) | 1035 (82.7) |
| cardiovascular | | | | | |
| risk factor (in | | | | | |
| addition to | | | | | |
| T2DM), n (%) | | | | | |
| Hypertension, n | 519 (55.4) | 655 (51.6) | 510 (51.0) | 1750 (52.1) | 688 (55.0) |
| (%) | | | | | |
| Hypercholesterolemia**, | 471 (50.3) | 565 (44.5) | 353 (35.3) | 1475 (44.0) | 566 (45.2) |
| n (%) | | | | | |
| Smoking history, | 383 (40.9) | 449 (35.4) | 393 (39.3) | 1301 (38.8) | 471 (37.6) |
| n (%) | | | | | |
| First degree | 190 (20.3) | 248 (19.5) | 186 (18.6) | 677 (20.2) | 265 (21.2) |
| relative with | | | | | |
| premature | | | | | |
| coronary heart | | | | | |
| disease, n (%) | | | | | |
| Total patient- | 1149 | 1462 | 1119 | 3758 | 1293 |
| years exposure | | | | | |
| Mean duration | 1.23 | 1.15 | 1.12 | 1.12 | 1.03 |
| of exposure, y | | | | | |

(a)Includes 20, 40 and 100 mg experience from CV181008
(b)Includes metformin monotherapy from CV181039
*CV history includes: previous MI, congestive heart failure, hospitalized for unstable angina, stable angina, percutaneous coronary intervention, coronary artery bypass graft, coronary artery disease, cerebrovascular disease, or peripheral vascular disease.
**Includes mixed dyslipidemia.

Table 5 shows overall exposure was 3758 patient-years on saxagliptin and 1293 patient-years on comparators. Within the saxagliptin population; 81% had at least one CV risk factor in addition to diabetes, with hypertension (52%), dyslipidemia (44%), or history of smoking (39%) the most common; 12% had known prior CV disease. Similar proportions were observed in the comparator group.

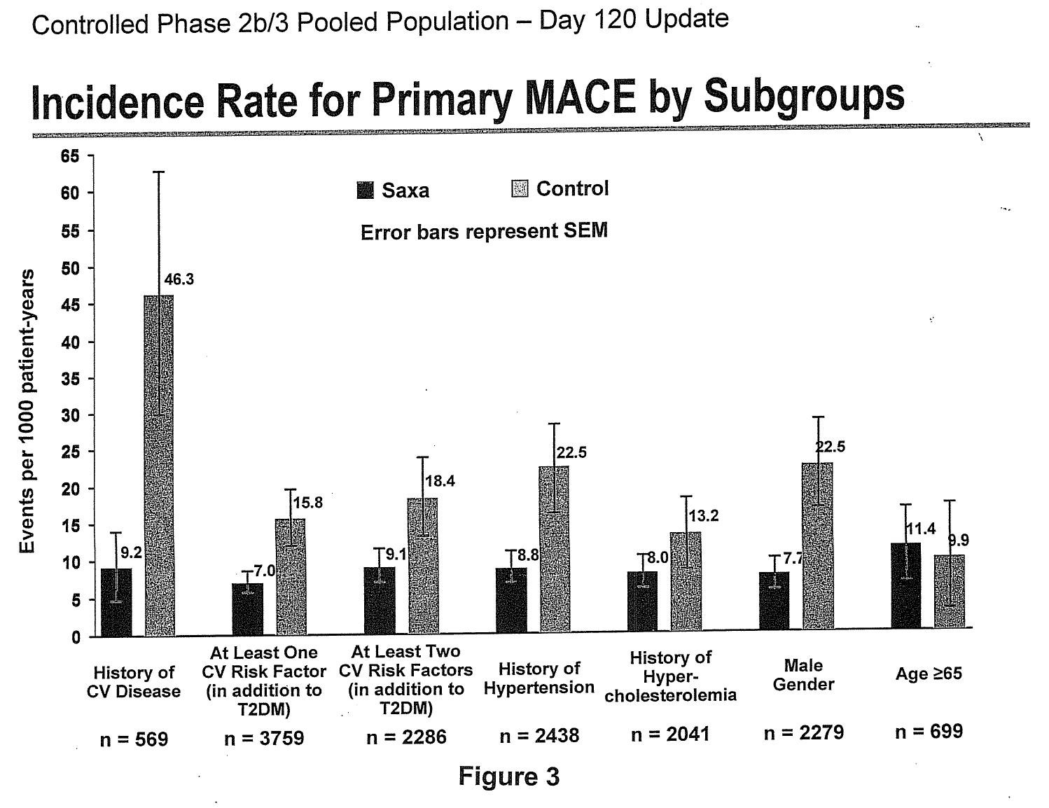

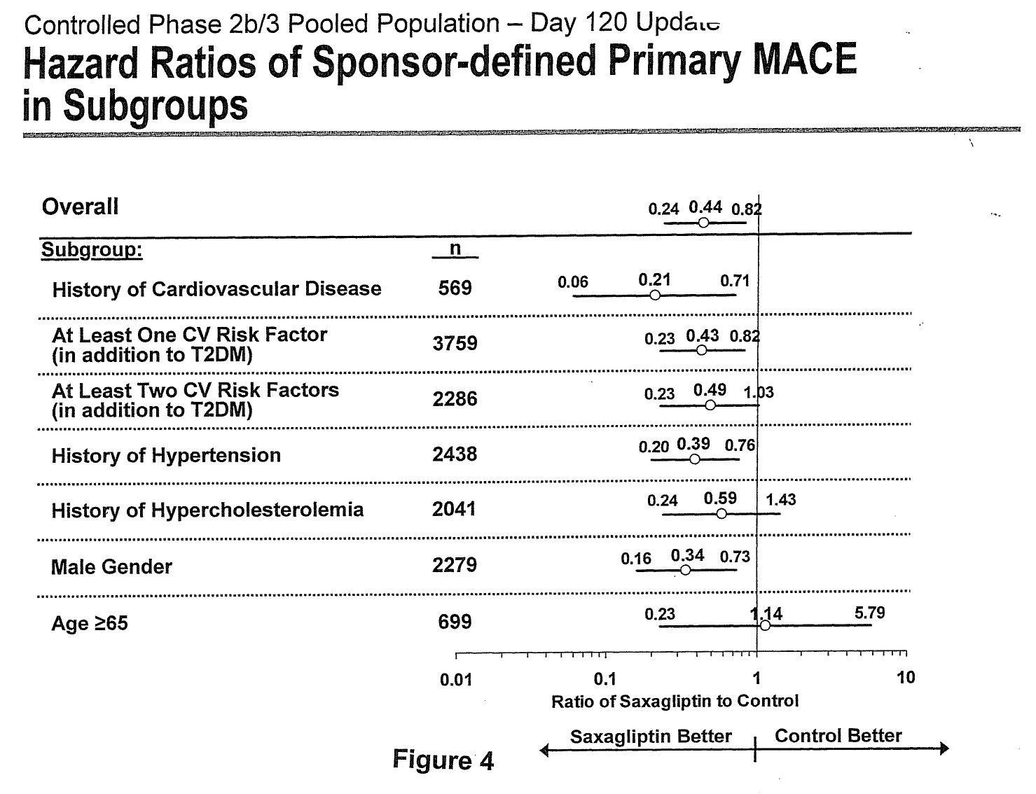

FIGS. 3 and 4 provide additional support that Phase 2b/3 subjects with a history of CV disease had lower incidence rates of primary MACE events when administered saxagliptin versus control. Subjects on saxagliptin had 9.2 MACE events per 1000 patient-years versus 46.3 MACE events per 1000 patient-years for control (FIG. 3). Saxagliptin also lowered incidence MACE events for subjects with: at least one CV risk factor (in addition to T2DM); at least two CV risk factors (in addition to T2DM); a history of hypertension; or a history of hypercholesterolemia, and in males (FIGS. 3—the event rate and 4—the relative risk). FIG. 4 discloses the data in terms of hazard ratios with a corresponding 95% confidence interval. For example, the point estimate for Phase 2b/3 subjects with a history of CV disease that were administered saxagliptin is 0.21. This means that during the time it would take 100 subjects in the comparator group (placebo or metformin administration) to have a MACE event, only 21 subjects in the saxagliptin group would have had a MACE event.

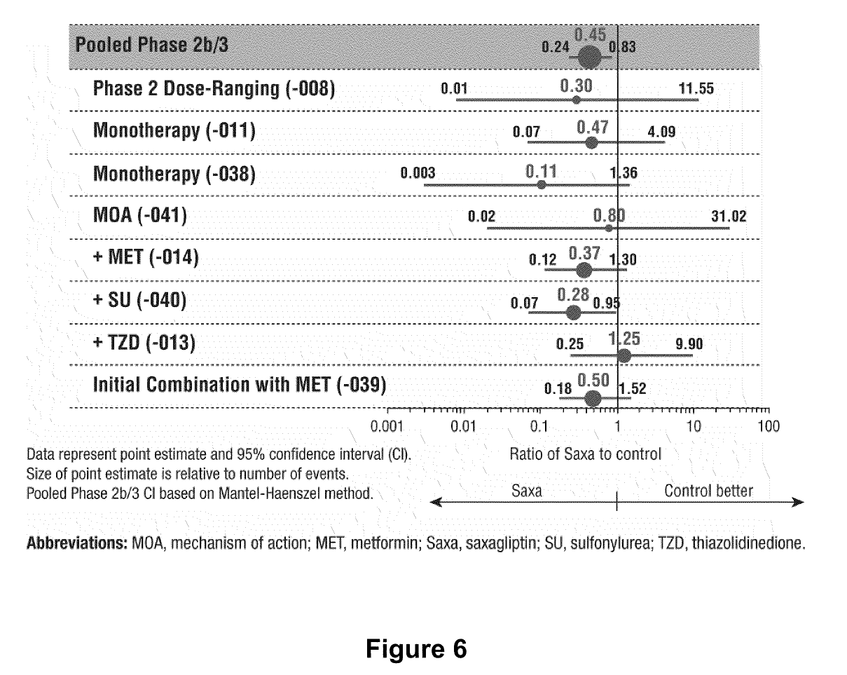

For the Phase 2b/3 pooled population, the overall incidence rate ratio based on a stratified Mantel-Haenszel approach for Primary MACE was 0.45 (0.24, 0.83) as shown in FIG. 6. This analysis indicates that when 100 people in the control group (placebo or metformin administration) had a MACE event, only 45 subjects administered saxagliptin will have had a MACE event (95% confidence). These results were consistent across the studies conducted with saxagliptin. In addition, FIG. 6 shows that saxagliptin in combination with a sulfonylurea or in combination with metformin reduces Primary MACE incidence rates.

More specifically, saxagliptin combined with metformin (FIG. 6, +MET) has a point estimate of 0.37. This suggests that when 100 subjects from the control group had a MACE event, only 37 subjects administered saxagliptin and metformin will have had a MACE event (95% confidence). The Phase 2b/3 studies were also conducted on subjects who were randomized to metformin or saxagliptin with or without metformin. MACE incidence rates were also reduced for these subjects (FIG. 6, initial Combination with Met) point estimate suggests when 100 subjects from the metformin only group had a MACE event only 50 subjects administered saxagliptin with or without metformin will have had a MACE event.

Further, MACE incidence rates were reduced for subjects administered saxagliptin and a sulfonylurea (FIG. 6, +SU) when 100 subjects from the control group had died, only 28 subjects administered saxagliptin and a sulfonylurea will have died. The 95% confidence interval suggests with 95% accuracy that at best when 100 subjects taking sulfonylurea have an event at best only 7 subjects taking sulfonylurea+saxagliptin and at worst 95 subjects taking sulfonylurea+saxagliptin will have had a MACE event.

An additional series of sensitivity analyses using related endpoints and alternative analytic methods produced consistent results:

First, two physicians at the Clinical Event Center (CEC) of the Duke Clinical Research Institute (Durham, N.C.) served as consultants to the sponsor and performed a retrospective treatment-blinded independent adjudication of all identified potential CV events. Reviewed cases included all deaths, MI, and stroke events as well as all events coded to any of the 148 PTs representing possible ischemic events from 2 MedDRA SMQs, “MI” and “central nervous system hemorrhages and cerebrovascular accidents”. Adjudication was based on definitions typical for such review and prespecified by the reviewers.

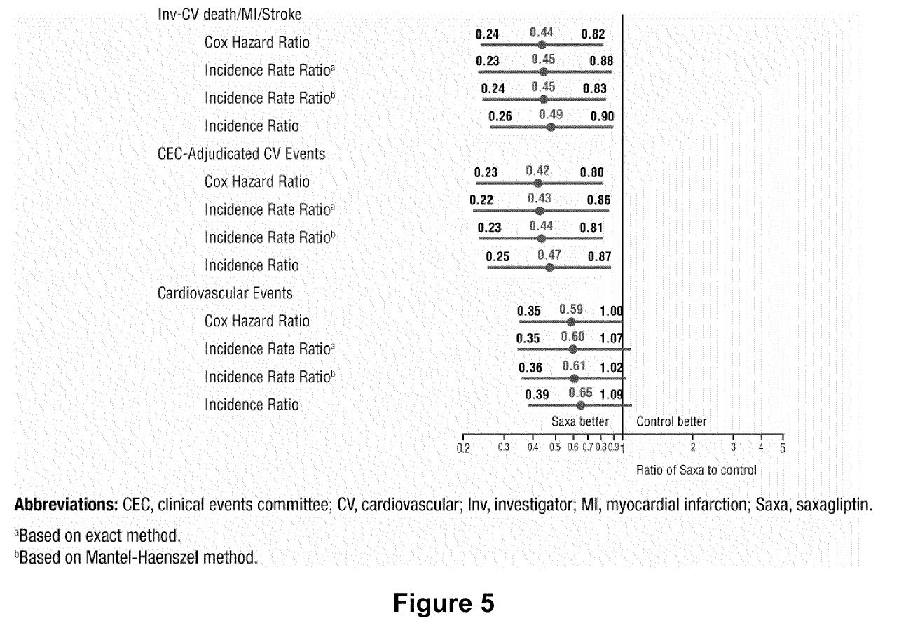

The events identified by this treatment blinded adjudication (CEC-CV death/MI/Stroke) were very consistent with the Inv-CV death/MI/stoke results (FIG. 5). The CV death assessment by the CEC was identical to the investigator based process (Table 3). The other event numbers and risk assessment of CEC-CV death/MI/Stroke were also very consistent with Inv-CV death/MI/stoke, Table 3 and FIG. 5 respectively. Both CEC and investigator analyses consistently suggested that patients on saxagliptin had a lower event rate for CV death/MI/stroke (MACE).

TABLE 6
Comparison Between the Investigator and
CEC Assessment of the CEC Reviewed Cases

| | |
|---|---|
| CV Death/MI/Stroke | Investigator Assessment |

| CEC Assessment | Identified | Not Identified |
|---|---|---|
| Yes | 38 | 2 |
| Unknown | 3 | 13 |
| No | 0 | 91 |

Abbreviations:
CEC, clinical events committee;
CV, cardiovascular;
MI, myocardial infarction

Table 6 categorizes in detail how the assessment of the blinded post-hoc adjudicated events compares to the investigator assessed events. There was complete concordance between the investigator/sponsor assessment and the CEC assessment of CV death. Ninety-three percent (38/41) of patients identified as Inv-CV death/MI/stroke were confirmed and included in CEC-CV death/MI/stroke. The 3 which were not “confirmed” were adjudicated as “unknown” due to inadequate documentation and, thus, not included in CEC-CV death/MI/stroke. Conversely there were 2 cerebrovascular events not identified as stroke in Inv-CV death/MI/stroke were adjudicated as stroke and were included in CEC-CV death/MI/stroke. Again, overall the blinded adjudicated events very closely paralleled the investigator assessment. This led to the quantitatively nearly identical analysis suggesting a reduced risk for CV death/MI/stroke events with saxagliptin treatment (FIG. 5).

Secondly, The CEC adjudication also allowed an assessment of the consistency across the pathologic components of CV death/MI/stoke (aka MACE, Table 3). As noted above the proportion of subjects with CV death was lower on Saxagliptin (0.2%) compared to controls (0.8%). For All Stroke (fatal and non-fatal) the proportion of subjects with events was 0.3% on Saxagliptin compared with 0.4% on controls. For all MI the proportion of subjects with events was 0.2% on Saxagliptin and 0.6% on controls. The proportion of subjects with events in the remaining category “Other CV death” was 0.1% on saxagliptin and 0.5% on comparator. This final category represents sudden deaths either not MI or stroke (1 saxagliptin subject with lung cancer with sudden death ascribed to pulmonary embolism, and 2 subjects on control with sudden death ascribed to congestive heart failure) or could not be confidently adjudicated as MI or stroke (3 on saxagliptin and 4 on comparitor). In summary each component of CV death/MI/stroke (MACE) was consistent with the overall assessment of saxagliptin to reduce CV death/MI/stroke events.

Third, much of congestive heart failure results from ischemic events. An effect on MACE events might be anticipated to result in fewer consequential CHF events. In fact the available data though limited is consistent with that expectation. Proportion of subjects with SAE CHF events was 0.2% on saxagliptin and 0.3% on comparator. The two fatal CHF events were on comparator, none was on saxagliptin.

In summary of the Saxagliptin clinical data, based on a >5000 patient-year clinical trial experience, there was no evidence of increased CV risk with saxagliptin treatment—as monotherapy or in combination with other oral antidiabetic agents. In fact the results consistently showed a reduction in events analyzed over many (related) endpoints: a) all death, b) CV death, c) Inv-CV death/MI/stroke (aka Primary MACE or MACE), d) CEC (adjudicated)-CV death/MI/stroke, e) Secondary MACE (all death/MI/Stroke), f) components of MACE (All stroke, All MI, “other CV death), g) cardiovascular events (ACE—includes reversible events and revascularization procedures), and h) CHF SAEs. For Inv-CV death/MI/Stroke there was consistent benefit suggested by a) multiple statistical methods, b) across multiple saxagliptin doses, c) across multiple studies, d) over time by Kaplan-Meier analysis, and e) across multiple high risk and low risk subpopulations. These data are interpreted to mean that saxagliptin has a cardioprotective effect.

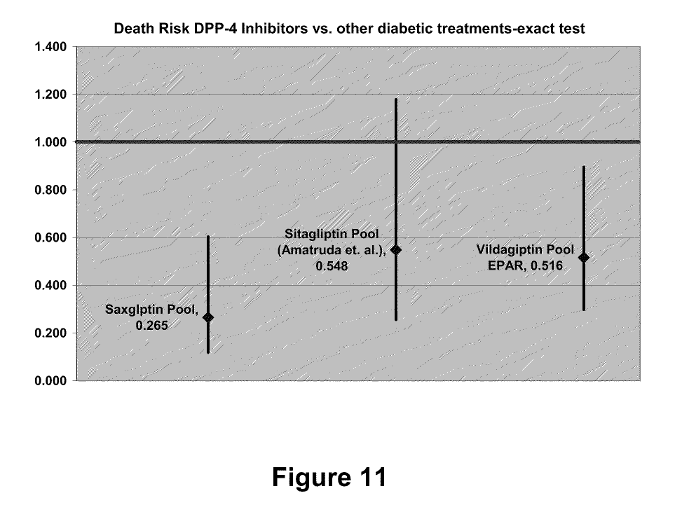

To evaluate whether the findings observed with saxagliptin are generalizable to the entire class of DPP-IV inhibitors, analyses were conducted on two known DPP-IV inhibitors, sitagliptin and vildagliptin, and their data was compared to saxagliptin. The sitagliptin data was available from a paper by Amatruda (et al.) pooling sitagliptin trials and the vildagliptin data came from the vildagliptin submission to the European Regulatory Agency. The results of the Fischer Exact Test analysis is shown in FIG. 11.

The analysis provided a “point estimate” of reduction in death for saxagliptin that means, for an equivalent population on saxagliptin, when 100 subjects on comparator treatment had died only 27 subjects on saxagliptin died. For the other DPP-4 inhibitors; sitagliptin that number would be 55 subjects and for vildagliptin 52 subjects. For saxagliptin the 95% confidence interval is very far from including one. One represents equal risk for subjects on saxagliptin and those not on saxagliptin. Thus as described earlier, we can be 95% confident, if the sample size included all subjects (the “true risk”) of the analyzed population, that fewer subjects would die in those treated with saxagliptin. The 95% confidence interval for vildagliptin also does not include one, but for sitagliptin it does include one. Again this means that if the sample size included all subjects of the analyzed population we can be 95% confident that fewer of them would die on vildagliptin. These results, shown in FIG. 11, provide support that DPP-IV inhibitors reduce the incidence of mortality. Further, the current evidence showed that saxagliptin, unexpectedly, is the most effective DPP-IV inhibitor in reducing incidence of mortality.

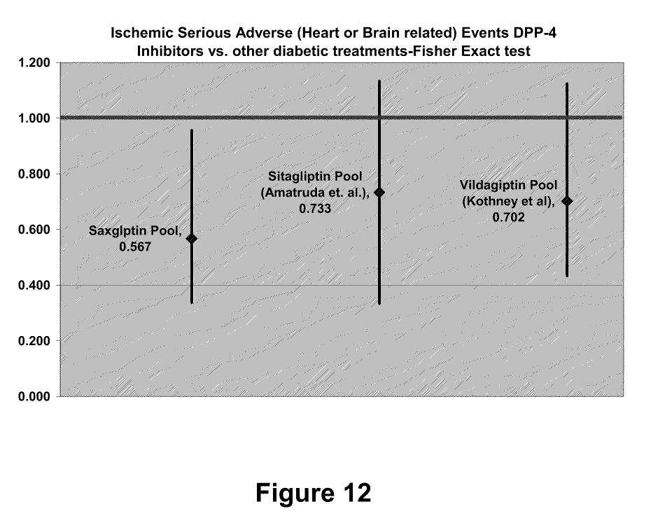

Additional analyses were conducted to determine whether DPP-4 inhibitors have benefits associated with heart attack and stroke-like events. The saxagliptin data was analyzed using criteria similar to two published studies for sitagliptin (Amatruda et. al.) and vildagliptin (Kothney et. al.). This endpoint was “ischemic” (events where the heart or brain is not getting enough blood) SAEs (death, hospitalization, or hospitalization narrowly prevented). The result for this analysis is shown in FIG. 12.

The results in FIG. 12 show that, for an equivalent group of subjects on saxagliptin, at the time when 100 subjects on comparator treatment had heart attack or stroke-like (including milder forms) events that would cause death, hospitalization, or near hospitalization, only 57 subjects on saxagliptin would have such events. For sitagliptin the number would be 73 and vildagliptin 70. Importantly, for saxagliptin the 95% confidence interval does not include 1 meaning that there is 95% confident that the “true” number, if the whole population were studied, would show a benefit for saxagliptin. All three DPP-4 inhibitors have a “best estimate” (point estimate) that supports a class effect of lowering incidence of heart attack and stroke. Saxagliptin, unexpectedly, has a superior effect in lowering incidence of heart attack and stroke as compared to sitagliptin and vildagliptin.

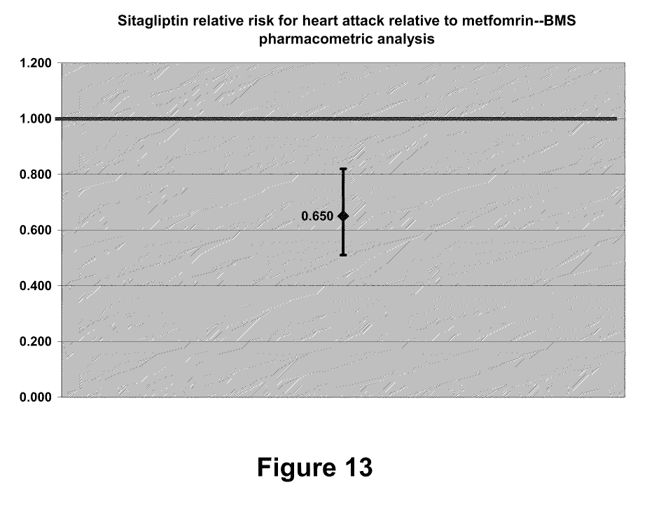

Because the analyses (FIGS. 11 and 12) for sitagliptin had 95% confidence intervals that included 1, an additional analysis was conducted on health insurance information from a single source to evaluate the potential benefits of sitagliptin. In particular, the health and risk factors of 27,424 subjects administered sitagliptin were compared to 80,462 subjects treated with metformin, the most common diabetes medication. It was discovered that those subjects who were treated with sitagliptin had substantially more risk factors than those treated with metformin. Despite that fact, the data showed there were fewer heart attacks on those treated with sitagliptin compared to those treated with metformin. The results of this analysis shows that, for a similar group treated with sitagliptin or metformin, in the time it takes 100 subjects to have a heart attack on metformin only 65 subjects will have had a heart attack on sitagliptin. Importantly in this analysis the 95% confidence interval does not include 1 (FIG. 13).

The analyses presented herein show that DPP-IV inhibitors lower the incidence of mortality. This effect appears to work through a reduction in heart attack and stroke like events. The data shows that for all three DPP-IV inhibitors analyzed at least one analysis has a 95% confidence interval that does not include 1. For only saxagliptin that is true for all of the analyses. These analyses show that saxagliptin has superior and unexpected properties relative to the other DPP-IV inhibitors for reducing the incidence of mortality and reducing the incidence of CV events.

With regard to combination therapy, the data disclosed herein provides evidence that DPP-IV inhibitors, particularly saxagliptin, in combination with metformin or a sulfonylurea, reduce incidence of mortality caused by any means, including by an adverse CV event, in humans. The evidence also shows that DPP-IV inhibitors, particularly saxagliptin, in combination with metformin or a sulfonylurea, reduce incidences of MACE in humans.

The beneficial effects of DPP-IV inhibitors, particularly saxagliptin, can be used to treat the general population, rather than only the diabetic population. The health benefits attributable to reduced glucose levels are realized over periods of time longer than the relatively short periods used to complete the Phase 2b/3 clinical studies presented herein. In addition, the United Kingdom Prospective Diabetes Study (UKPDS) and the Diabetes Control and Complications Trial (DCCT) extensions make it clear that it takes approximately a decade of improved glycemic control before reductions in glucose result in a reduced risk of death or cardiovascular events. As shown in FIG. 1 and in the other saxagliptin, sitagliptin, and vildagliptin (for the latter two most studies were less than a year duration) analysis the influence of DPP-IV on death and CV events occurs within the first year and likely in the first 6 months. This strongly shows that the effect is not related to glucose. Since the effect is not related to glucose control, but does appear to be more evident in subgroups of higher risk, it is the risk factor, not whether the subject's glucose is normal (non-diabetic) or elevated (diabetic) which should serve as the basis for treatment. The available data thus apply to both diabetic and non-diabetic populations.

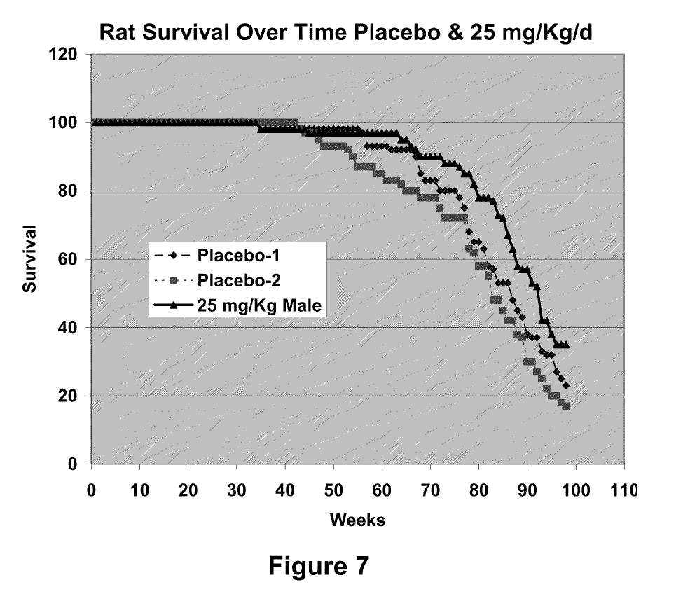

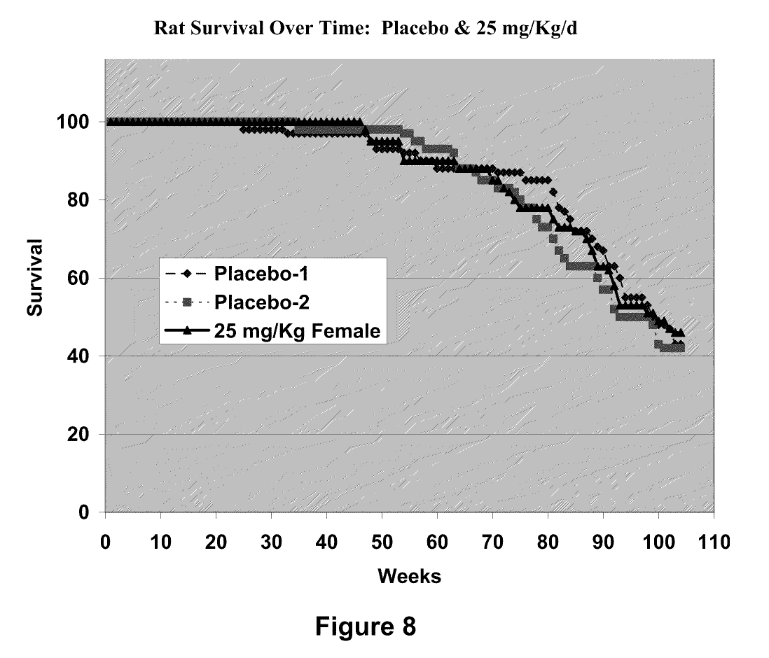

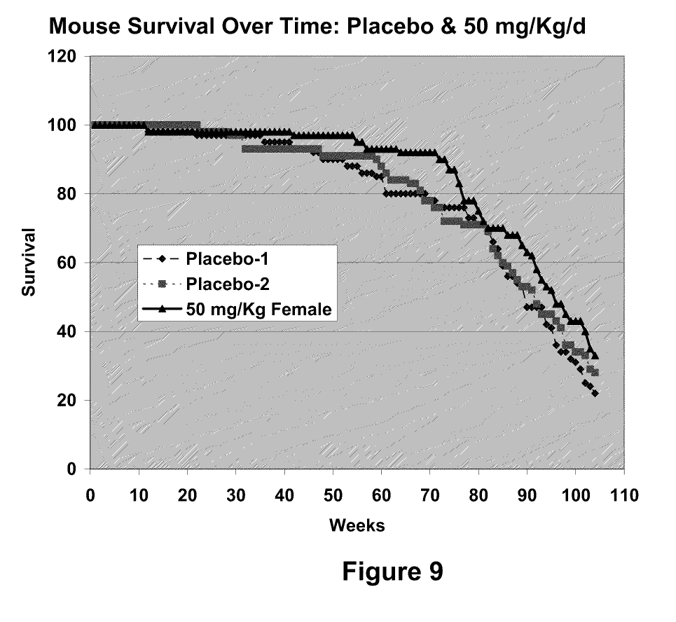

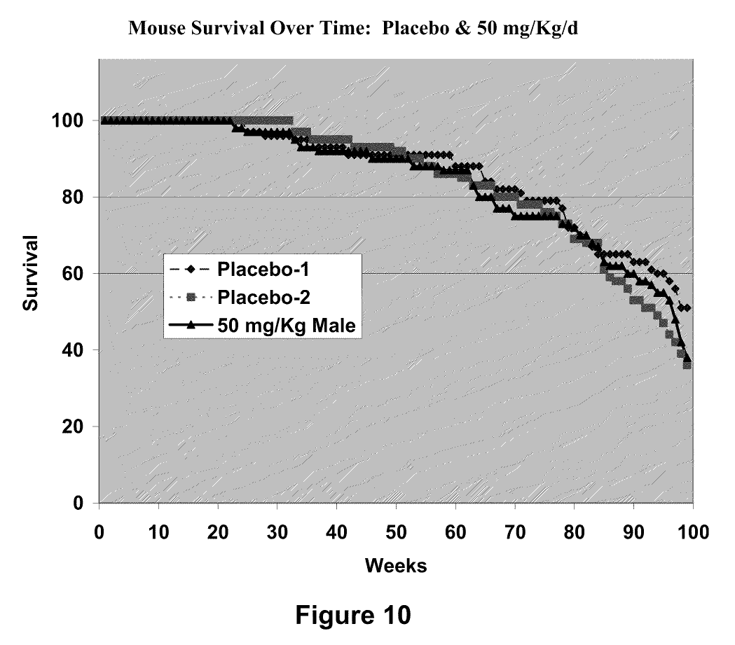

FIGS. 7-10 illustrate preclinical data that supports reduced risk of mortality in non-diabetic rats and mice, mammals other than humans, following chronic administration of the DPP-IV inhibitor saxaglitpin. In these studies, approximately sixty animals of each gender were exposed to saxagliptin or two cohorts of placebo over two years, the approximate lifetime of a rodent. Male rats exposed to 25 mg/kg/day of saxagliptin (FIG. 7) showed a reduced risk of mortality or longer survival times than rats administered placebo. Females rats exposed to the same dose (FIG. 8) appeared intermediate between the two placebo cohorts. With regard to mice, females exposed to 50 mg/kg/day of saxagliptin (FIG. 9) showed a reduced risk of mortality compared to placebo, while the males exposed to this dose (FIG. 10) appeared intermediate between the two placebo cohorts. The differences in survival time between male and female rodents are not currently explained. A possible explanation for the rat results is that the dose taken by male rats, 45 times the human equivalent dose, was closer to the human experience than the same dose taken by female rats, 112 times the human equivalent dose, and therefore the male experience may be more relevant. Another possible explanation is that male rats are known to succumb to chronic progressive nephropathy as they age, female rats essentially do not. Overall, these findings support an effect of saxagliptin consistent with reducing risk of all cause mortality in mammals other than humans, including mammals that are not diabetic.

We claim:

1. A method of reducing the risk of mortality in a mammal, particularly a human, comprising administering to the mammal or human, in need of such treatment, a therapeutically effective amount of a DPP-IV inhibitor.

2. The method according to claim 1 wherein the DPP-IV inhibitor is saxagliptin.

3. The method according to claim 2 wherein the therapeutically effective amount is about 0.5 mgs/day to about 400 mgs/day.

4. The method according to claim 2 wherein the therapeutically effective amount is about 2.5 mgs/day, about 5.0 mgs/day, or about 10 mgs/day.

5. The method according to claim 1 wherein the DPP-IV inhibitor is vildagliptin.

6. The method according to claim 5 wherein the therapeutically effective amount is 25 mgs/day to 100 mgs/day given once daily or in divided doses.

7. The method according to claim 1 wherein the DPP-IV inhibitor is sitagliptin.

8. The method according to claim 7 wherein the therapeutically effective amount is 25 mgs/day to 100 mgs/day given once daily or in divided doses.

9. The method according to claim 1 wherein the human has a history of CV disease, hypertension, hypercholesterolemia, mixed dyslipidemia, or a smoking history (current/previous).

10. The method according to claim 1 wherein the human has suffered a previous cardiovascular event.

11. A method of reducing the risk of non-fatal myocardial infarction, non-fatal stroke, or CV death in a mammal, particularly a human, comprising administering to the mammal or human, in need of such treatment, a therapeutically effective amount of a DPP-IV inhibitor.

12. The method according to claim 11 wherein the DPP-IV inhibitor is saxagliptin.

13. The method according to claim 12 wherein the therapeutically effective amount is about 0.5 mgs/day to about 400 mgs/day.

14. The method according to claim 12 wherein the therapeutically effective amount is about 2.5 mgs/day, about 5.0 mgs/day, or about 10 mgs/day.

15. The method according to claim 11 wherein the DPP-IV inhibitor is vildagliptin.

16. The method according to claim 15 wherein the therapeutically effective amount is 25 mgs/day to 100 mgs/day given once daily or in divided doses.

17. The method according to claim 11 wherein the DPP-IV inhibitor is sitagliptin.

18. The method according to claim 17 wherein the therapeutically effective amount is 25 mgs/day to 100 mgs/day given once daily or in divided doses.

19. The method according to claim 11 wherein the human has a history of CV disease, hypertension, hypercholesterolemia, mixed dyslipidemia, or a smoking history (current/previous).

20. The method according to claim 11 wherein the human has suffered a previous cardiovascular event.
